(19) United States
(12) Patent Application Publication
(10) Pub. No.: US 2022/0203347 A1
(43) Pub. Date: Jun. 30, 2022

(54) METHOD FOR REGENERATION OF AN OVERLOAD CHROMATOGRAPHY COLUMN

(71) Applicant: Genentech, Inc., South San Francisco, CA (US)

(72) Inventors: Marc L. Wong, San Carlos, CA (US); Donna F. Quicho, San Francisco, CA (US); Jason A. Brown, San Ramon, CA (US); Josefine K. Persson, Half Moon Bay, CA (US); Amy Lim, Palo Alto, CA (US)

(73) Assignee: Genentech, Inc., South San Francisco, CA (US)

(21) Appl. No.: 17/542,138

(22) Filed: Dec. 3, 2021

Related U.S. Application Data
(63) Continuation of application No. PCT/US2020/036126, filed on Jun. 4, 2020.
(60) Provisional application No. 62/857,734, filed on Jun. 5, 2019.

(51) Int. Cl. B01J 49/53; B01J 41/20; B01J 49/07; B01J 49/57; C07K 1/18; B01J 49/06; B01J 39/26; B01D 15/36; B01D 15/20; B01D 15/42; B01D 15/38

(52) U.S. Cl. CPC B01J 49/53; B01J 41/20; B01J 49/07; B01J 49/57; C07K 1/18; B01D 15/3809; B01J 39/26; B01D 15/362; B01D 15/203; B01D 15/42; B01D 15/363; B01J 49/06

(57) ABSTRACT

The present invention provides methods for cleaning or regenerating a chromatography material for reuse. The methods of the invention can be used for cleaning or regenerating ion exchange chromatography columns for reuse in the large-scale manufacture of multiple polypeptide products where the ion exchange chromatography column in used in overload mode.

### CROSS-REFERENCE TO RELATED APPLICATIONS

This application claims the priority benefit of U.S. Provisional Application No. 62/857,734, filed Jun. 5, 2019, which is incorporated herein by reference in its entirety.

### FIELD OF THE INVENTION

The present invention provides methods for chromatography.

### BACKGROUND OF THE INVENTION

Overload (OL) cation-exchange chromatography has been shown to have equivalent impurity removal capabilities to bind and clutch mode while intensifying the process to improve resin utilization and plant efficiency. The resin is loaded under strong binding conditions beyond the resin dynamic binding capacity. Impurities preferentially bind to the resin while the product breakthrough is collected. Due to the high load density (typically >500 g/L), a higher concentration of host-cell protein remains bound to the resin. As such, the column fouls (high column pressure, >80 psi) during the sanitization phase (0.5N Sodium Hydroxide) of the purification process (FIG. 1). What is needed is as improved process that minimizes pressure increases during the sanitization phase with no impurity carry-over between chromatography runs.

Methods of overload chromatography are described in US Patent Application Publications US 2013/0079272A1 and US 2014/0301977A1, each of which is incorporated by reference herein in its entirety.

All references cited herein, including patent applications and publications, are incorporated by reference in their entirety.

### BRIEF SUMMARY

In some aspects, the invention provides methods to clean an ion exchange chromatography material (e.g., a cation exchange chromatography material) for reuse wherein the chromatography material is used in overload mode in the purification of a polypeptide, the method comprising the steps of a) passing a regeneration buffer through the ion exchange chromatography material, wherein the regeneration buffer comprises sodium acetate; b) passing of sanitization buffer through the ion exchange chromatography material, wherein the sanitization buffer comprises about 0.5 N NaOH; and c) storing the ion exchange chromatography material in a storage buffer, wherein the storage buffer comprises about 0.1 N NaOH. In some embodiments, the regeneration buffer comprises about 50 to about 600 mM sodium acetate (NaOAc). In some embodiments, the regeneration buffer comprises about 350 mM NaOAc. In some embodiments, the pH of the regeneration buffer is about pH 4.0 to 11. In some embodiments, the pH of the regeneration buffer is about pH 8.3. In some embodiments, about 3 to about 12 material volumes of regeneration buffer are passed through the ion exchange chromatography material. In some embodiments, about 10 material volumes of regeneration buffer are passed through the ion exchange chromatography material. In some embodiments, about 2 to about 12 material volumes of sanitization buffer are passed through the ion exchange chromatography material. In some embodiments, about 10 material volumes of sanitization buffer are passed passing a regeneration buffer through the ion exchange chromatography material. In some embodiments, the ion exchange chromatography material is stored in about 1 to 5 material volumes of storage buffer. In some embodiments, the ion exchange chromatography material is stored in about 4 material volumes of storage buffer.

In some aspects, the invention provides a method to clean an ion exchange chromatography material for reuse wherein the chromatography material is used in overload mode in the purification of a polypeptide, the method comprising the steps of a) passing a regeneration buffer through the ion exchange chromatography material, wherein the regeneration buffer comprises sodium acetate; b) passing an acid wash through the ion exchange chromatography material, c) passing of sanitization buffer through the ion exchange chromatography material, wherein the sanitization buffer comprises about 0.5 N NaOH and about 1 M NaCl; and d) storing the ion exchange chromatography material in a storage buffer, wherein the storage buffer comprises about 0.1 N NaOH. In some embodiments, the regeneration buffer and the second regeneration buffer comprises about 25 to about 600 mM NaOAc. In some embodiments, the regeneration buffer comprises about 350 mM NaOAc. In some embodiments, the regeneration buffer comprises about 40 mM NaOAc. In some embodiments, the pH of the regeneration buffer is about pH 2.0 to 7.0. In some embodiments, the pH of the regeneration buffer is about pH 4.0. In some embodiments, the pH of the regeneration buffer is about pH 5.0. In some embodiments, about 3 to about 12 material volumes of regeneration buffer are passed through the ion exchange chromatography material. In some embodiments, about 7 material volumes of regeneration buffer are passed through the ion exchange chromatography material. In some embodiments, the acid wash is about 167M NaOAc and about 0.3M phosphoric acid. In some embodiments, about 1 to about 5 material volumes of the acid wash are passed through the ion exchange chromatography material. In some embodiments, about 3 material volumes of the acid wash are passed through the ion exchange chromatography material. In some embodiments, about 2 to about 12 material volumes of sanitization buffer are passed through the ion exchange chromatography material. In some embodiments, about 4 material volumes of sanitization buffer are passed passing a regeneration buffer through the ion exchange chromatography material. In some embodiments, the ion exchange chromatography material is stored in about 1 to 5 material volumes of storage buffer. In some embodiments, the ion exchange chromatography material is stored in about 3 material volumes of storage buffer.

In some aspects, the invention provides methods to clean a ion exchange chromatography material (e.g., a cation exchange chromatography material) for reuse wherein the chromatography material is used in overload mode in the purification of a polypeptide, the method comprising the steps of a) passing a regeneration buffer through the ion exchange chromatography material, wherein the regeneration buffer comprises Tris and sodium acetate, b) passing a sanitization buffer through the ion exchange chromatography material, wherein the sanitization buffer comprises about 0.5 N NaOH; and c) storing the ion exchange chromatography material in a storage buffer, wherein the storage buffer comprises about 0.1 N NaOH. In some embodiments, the regeneration buffer comprises about 25 to about 75 mM Tris and about 50 to about 100 mM NaOAc. In some embodiments, the regeneration buffer comprises about 50 mM Tris and about 85 mM NaOAc. In some embodiments, the pH of the regeneration buffer is about pH 7 to 9. In some embodiments, the pH of the regeneration buffer is about pH 8.0. In some embodiments, about 3 to about 12 material volumes of regeneration buffer are passed through the ion exchange chromatography material. In some embodiments, about 10 material volumes of regeneration buffer are passed through the ion exchange chromatography material. In some embodiments, about 2 to about 12 material volumes of sanitization buffer are passed through the ion exchange chromatography material. In some embodiments, about 10 material volumes of sanitization buffer are passed passing a regeneration buffer through the ion exchange chromatography material. In some embodiments, the ion exchange chromatography material is stored in about 1 to 5 material volumes of storage buffer. In some embodiments, the ion exchange chromatography material is stored in about 4 material volumes of storage buffer.

In some aspects, the invention provides methods for purifying a polypeptide from a composition comprising the polypeptide and an impurity using a ion exchange chromatography material (e.g., a cation exchange chromatography material) wherein the ion exchange chromatography material is suitable for reuse, the method comprising the steps of a) equilibrating an ion exchange chromatography material with an equilibration buffer; b) loading the composition on the ion exchange chromatography material, wherein the load density is ≤about 1000 g/L; c) collecting fractions comprising the polypeptide; d) washing the ion exchange chromatography material with equilibration buffer; e) passing a regeneration buffer through the ion exchange chromatography material, wherein the regeneration buffer comprises sodium acetate; f) passing of sanitization buffer through the ion exchange chromatography material, wherein the sanitization buffer comprises about 0.5 N NaOH; and g) storing the ion exchange chromatography material in a storage buffer, wherein the storage buffer comprises about 0.1 N NaOH. In some embodiments, the equilibration buffer comprises about 20 mM to about 60 mM sodium acetate. In some embodiments, the equilibration buffer comprises about 40 mM sodium acetate. In some embodiments, the pH of the equilibration buffer is about pH 5.0 to about pH 6.0. In some embodiments, the pH of the equilibration buffer is about pH 5.5. In some embodiments, the ion exchange chromatography material is washed with about 3-7 material volumes of equilibration buffer. In some embodiments, the ion exchange chromatography material is washed with about 5 material volumes of equilibration buffer. In some embodiments, the regeneration buffer comprises about 50 to about 600 mM NaOAc. In some embodiments, the regeneration buffer comprises about 350 mM NaOAc. In some embodiments, the pH of the regeneration buffer is about pH 4.0 to 11. In some embodiments, the pH of the regeneration buffer is about pH 8.3. In some embodiments, about 3 to about 12 material volumes of regeneration buffer am passed through the ion exchange chromatography material. In some embodiments, about 10 material volumes of regeneration buffer are passed through the ion exchange chromatography material. In some embodiments, about 2 to about 12 material volumes of sanitization buffer are passed through the ion exchange chromatography material. In some embodiments, about 10 material volumes of sanitization buffer are passed passing a regeneration buffer through the ion exchange chromatography material. In some embodiments, the ion exchange chromatography material is stored in about 1 to 5 material volumes of storage buffer. In some embodiments, the ion exchange chromatography material is stored in about 4 material volumes of storage buffer.

In some aspects, the invention provides a method for purifying a polypeptide from a composition comprising the polypeptide and an impurity using an ion exchange chromatography material wherein the ion exchange chromatography material is suitable for reuse, the method comprising the steps of a) equilibrating an ion exchange chromatography material with an equilibration buffer; b) loading the composition on the ion exchange chromatography material, wherein the load density exceeds the dynamic binding capacity of the ion exchange chromatography for the polypeptide; c) collecting fractions comprising the polypeptide; d) washing the ion exchange chromatography material with equilibration buffer; e) passing a regeneration buffer through the ion exchange chromatography material, wherein the regeneration buffer comprises sodium acetate; f) passing an acid wash through the ion exchange chromatography material, g) passing a sanitization buffer through the ion exchange chromatography material, wherein the sanitization buffer comprises about 0.5 N NaOH and about 1 M NaCl; and h) storing the ion exchange chromatography material in a storage buffer, wherein the storage buffer comprises about 0.1 N NaOH. In some embodiments, the load density is ≤about 1000 g/L. In some embodiments, the load density is ≥about 20 g/L. In some embodiments, the load density is ≥about 70 g/L. In some embodiments, the load density is at least about 2× the dynamic binding capacity of the ion exchange chromatography material for the polypeptide. In some embodiments, the load density is about 2× to about 100× the dynamic binding capacity of the ion exchange chromatography material for the polypeptide. In some embodiments, the load density is about 10× the dynamic binding capacity of the ion exchange chromatography material for the polypeptide. In some embodiments, the equilibration buffer comprises about 50 mM to about 100 mM NaOAc and about 25 mM to about 75 mM Tris. In some embodiments, the equilibration buffer comprises about 85 mM sodium acetate and about 50 mM Tris. In some embodiments, the pH of the equilibration buffer is about pH 6.0 to about pH 10.0. In some embodiments, the pH of the equilibration buffer is about pH 8.0. In some embodiments, the ion exchange chromatography material is washed with about 5 to about 15 material volumes of equilibration buffer. In some embodiments, the ion exchange chromatography material is washed with about 10 material volumes of equilibration buffer. In some embodiments, the regeneration buffer comprises about 25 to about 600 mM NaOAc. In some embodiments, the regeneration buffer comprises about 350 mM NaOAc. In some embodiments, the regeneration buffer comprises about 40 mM NaOAc. In some embodiments, the pH of the regeneration buffer is about pH 2.0 to 7.0. In some embodiments, the pH of the regeneration buffer is about pH 4.0. In some embodiments, the pH of the regeneration buffer is about pH 5.0. In some embodiments, about 3 to about 12 material volumes of the regeneration buffer are passed through the ion exchange chromatography material. In some embodiments, about 7 material volumes of the regeneration buffer are passed through the ion exchange chromatography material. In some embodiments, the acid wash is about 167M NaOAc and about 0.3M phosphoric acid. In some embodiments, about 1 to about 5 material volumes of the acid wash are passed through the ion exchange chromatography material. In some embodiments, about 3 material volumes of the acid wash are passed through the ion exchange chromatography material. In some embodiments, about 2 to about 12 material volumes of sanitization buffer are passed through the ion exchange chromatography material. In some embodiments, about 4 material volumes of sanitization buffer are passed passing a regeneration buffer through the ion exchange chromatography material. In some embodiments, the ion exchange chromatography material is stored in about 1 to 5 material volumes of storage buffer. In some embodiments, the ion exchange chromatography material is stored in about 3 material volumes of storage buffer.

In some aspects, the invention provides methods for purifying a polypeptide from a composition comprising the polypeptide and an impurity using an ion exchange chromatography material wherein the ion exchange chromatography material is suitable for reuse, the method comprising the steps of a) equilibrating the ion exchange chromatography material with an equilibration buffer; b) loading the composition on the ion exchange chromatography material, wherein the load density is ≤about 1000 g/L; c) collecting fractions comprising the polypeptide; d) washing the ion exchange chromatography material with equilibration buffer; e) passing a regeneration buffer through the ion exchange chromatography material, wherein the regeneration buffer comprises Tris and sodium acetate; f) passing a sanitization buffer through the ion exchange chromatography material, wherein the sanitization buffer comprises about 0.5 N NaOH; and g) storing the ion exchange chromatography material in a storage buffer, wherein the storage buffer comprises about 0.1 N NaOH.

In some aspects, the invention provides methods for purifying a polypeptide from a composition comprising the polypeptide and an impurity using an ion exchange chromatography material wherein the ion exchange chromatography material is suitable for reuse, the method comprising the steps of a) equilibrating an ion exchange chromatography material with an equilibration buffer; b) loading the composition on the ion exchange chromatography material, wherein the load density is ≥20 g/L; c) collecting fractions comprising the polypeptide; d) washing the ion exchange chromatography material with equilibration buffer; c) passing a regeneration buffer through the ion exchange chromatography material, wherein the regeneration buffer comprises sodium acetate; f) passing of sanitization buffer through the ion exchange chromatography material, wherein the sanitization buffer comprises about 0.5 N NaOH; and g) storing the ion exchange chromatography material in a storage buffer, wherein the storage buffer comprises about 0.1 N NaOH.

In some embodiments, the load density is ≥70 g/L. In some aspects, the invention provides methods for purifying a polypeptide from a composition comprising the polypeptide and an impurity using an ion exchange chromatography material wherein the ion exchange chromatography material is suitable for reuse, the method comprising the steps of a) equilibrating the ion exchange chromatography material with an equilibration buffer; b) loading the composition on the ion exchange chromatography material, wherein the load density is ≥20 g/L; c) collecting fractions comprising the polypeptide; d) washing the ion exchange chromatography material with equilibration buffer; e) passing a regeneration buffer through the ion exchange chromatography material, wherein the regeneration buffer comprises Tris and sodium acetate; f) passing a sanitization buffer through the ion exchange chromatography material, wherein the sanitization buffer comprises about 0.5 N NaOH; and g) storing the ion exchange chromatography material in a storage buffer, wherein the storage buffer comprises about 0.1 N NaOH.

In some aspects, the invention provides methods for purifying a polypeptide from a composition comprising the polypeptide and an impurity using an ion exchange chromatography material wherein the ion exchange chromatography material is suitable for reuse, the method comprising the steps of a) equilibrating an ion exchange chromatography material with an equilibration buffer; b) loading the composition on the ion exchange chromatography material, wherein the load density exceeds the dynamic binding capacity of the ion exchange chromatography for the polypeptide; c) collecting fractions comprising the polypeptide; d) washing the ion exchange chromatography material with equilibration buffer; e) passing a regeneration buffer through the ion exchange chromatography material, wherein the regeneration buffer comprises sodium acetate; f) passing of sanitization buffer through the ion exchange chromatography material, wherein the sanitization buffer comprises about 0.5 N NaOH; and g) storing the ion exchange chromatography material in a storage buffer, wherein the storage buffer comprises about 0.1 N NaOH. In some embodiments, the load density is at least about 2× the dynamic binding capacity of the ion exchange chromatography material for the polypeptide. In some embodiments, the load density is about 2× to about 100× the dynamic binding capacity of the ion exchange chromatography material for the polypeptide. In some embodiments, the load density is about 10× the dynamic binding capacity of the ion exchange chromatography material for the polypeptide.

In some aspects, the invention provides methods for purifying a polypeptide from a composition comprising the polypeptide and an impurity using an ion exchange chromatography material wherein the ion exchange chromatography material is suitable for reuse, the method comprising the steps of a) equilibrating the ion exchange chromatography material with an equilibration buffer; b) loading the composition on the ion exchange chromatography material, wherein the load density exceeds the dynamic binding capacity of the ion exchange chromatography for the polypeptide; c) collecting fractions comprising the polypeptide; d) washing the ion exchange chromatography material with equilibration buffer; e) passing a regeneration buffer through the ion exchange chromatography material, wherein the regeneration buffer comprises Tris and sodium acetate; f) passing a sanitization buffer through the ion exchange chromatography material, wherein the sanitization buffer comprises about 0.5 N NaOH; and g) storing the ion exchange chromatography material in a storage buffer, wherein the storage buffer comprises about 0.1 N NaOH.

In some embodiments of the above aspects, the equilibration buffer comprises about 20 mM to about 60 mM sodium acetate. In some embodiments, the equilibration buffer comprises about 40 mM sodium acetate. In some embodiments, the pH of the equilibration buffer is about pH 5.0 to about pH 6.0. In some embodiments, the pH of the equilibration buffer is about pH 5.5. In some embodiments, the ion exchange chromatography material is washed with about 3-7 material volumes of equilibration buffer. In some embodiments, the ion exchange chromatography material is washed with about 5 material volumes of equilibration buffer. In some embodiments, the regeneration buffer comprises about 25 to about 75 mM Tris and about 50 to about 100 mM NaOAc. In some embodiments, the regeneration buffer comprises about 50 mM Tris and about 85 mM NaOAc. In some embodiments, the pH of the regeneration buffer is about pH 7 to 9. In some embodiments, the pH of the regeneration buffer is about pH 8.0. In some embodiments, about 3 to about 12 material volumes of regeneration buffer are passed through the ion exchange chromatography material. In some embodiments, about 10 material volumes of regeneration buffer are passed through the ion exchange chromatography material. In some embodiments, about 2 to about 12 material volumes of sanitization buffer are passed through the ion exchange chromatography material, in some embodiments, about 10 material volumes of sanitization buffer are passed passing a regeneration buffer through the ion exchange chromatography material. In some embodiments, the ion exchange chromatography material is stored in about 1 to 5 material volumes of storage buffer. In some embodiments, the ion exchange chromatography material is stored in about 4 material volumes of storage buffer.

In some embodiments of the invention, the ion exchange chromatography material is in a chromatography column. In some embodiments, the ion exchange chromatography material is a cation exchange chromatography material. In some embodiments, the ion exchange chromatography material comprises a sulfopropyl moiety linked to a support matrix. In some embodiments, the support matrix comprises cross-linked poly(styrenedivinylbenzene). In some embodiments, the ion exchange chromatography material is a POROS™ HS50 material. In some embodiments, the ion exchange chromatography material is a mixed mode cation exchange material. In some embodiments, the mixed mode cation exchange material is Capto MMC™, Capto MMC™ ImpRes, Nuvia™ cPrime™, or Toyopearl MX Trp-650M.

In some embodiments of the invention, the ion exchange chromatography material is an anion exchange chromatography material. In some embodiments, the ion exchange chromatography material comprises a primary amine, a secondary amine, a tertiary amine or a quaternary ammonium ion functional group, a polyamine functional group, or a diethylaminoethyl functional group linked to a support matrix. In some embodiments, the ion exchange chromatography material is a mixed mode anion exchange material. In some embodiments, the mixed mode anion exchange material is Capto Adhere™ or Capto Adhere™ ImpRes.

In some embodiments of the invention, the ion exchange chromatography material is used for large-scale production of the polypeptide. In some embodiments, the polypeptide is an antibody, an immunoadhesin, an Fc-containing protein, or an immunoconjugate. In some embodiments, the polypeptide is an antibody. In some embodiments, the antibody is a monoclonal antibody. In some embodiments, the monoclonal antibody is a chimeric antibody, humanized antibody, or human antibody. In some embodiments, the monoclonal antibody is an IgG monoclonal antibody. In some embodiments, the antibody is an antigen binding fragment. In some embodiments, the antigen binding fragment is a Fab fragment, a Fab′ fragment, a F(ab′)2 fragment, a scFv, a di-scFv, a bi-scFv, a tandem (di, tri)-scFv, a Fv, a sdAb, a tri-functional antibody, a BiTE, a diabody or a triabody. In some embodiments, the antibody is a bispecific antibody. In some embodiments, the antibody is selected from the group consisting of an anti-CD20 antibody, an anti-CD40 antibody, an anti-HER2 antibody, an anti-IL6 antibody, an anti-IgE antibody, an anti-IL13 antibody, an anti-Flu A antibody, an anti-TIGIT antibody, an anti-PD-L1 antibody, an anti-VEGF-A antibody, an anti-VEGF-A/ANG2 antibody, an anti-CD79b antibody, an anti-ST2 antibody, an anti-factor D antibody, an anti-factor IX antibody, an anti-factor X antibody, an anti-abeta antibody, an anti-tau antibody, an anti-CEA antibody, an anti-CEA/CD3 antibody, an anti-CD20/CD3 antibody, an anti-FcRH5/CD3 antibody, an anti-Her2/CD3 antibody, an anti-FGFR1/KLB antibody, a FAP-4-1 BBL fusion protein, and a FAP-IL2v fusion protein. In some embodiments, the antibody is selected from the group consisting of ocrelizumab, pertuzumab, trastuzumab, tocilizumab, faricimab, polatuzumab, gantenerumab, cibisatamab, crenezumab, mosunetuzumab, tiragolumab, bevacizumab, rituximab, atezolizumab, obinutuzumab, lampalizumab, lebrikizumab, omalizumab ranibizumab, emicizumab, selicrelumab, prasinezumab, RO6874281 and RO7122290.

In some embodiments of the invention, the polypeptide is purified using an affinity chromatography material prior to passing through the ion exchange chromatography material, wherein the ion exchange chromatography material is a cation exchange chromatography material. In some embodiments, the affinity material is selected from the group consisting of a Protein A chromatography, a Protein G chromatography, a Protein A/G chromatography, a protein L chromatography, a FcXL chromatography, a protein XL chromatography, a kappa chromatography, and a kappaXL chromatography. In some embodiments, the Protein A affinity material is a MAbSelect material, a MAbSelect SuRe™ material or a MAbSelect SuRe™ LX material. In some embodiments, the buffers are passed through the ion exchange chromatography material at about 15-20 material volumes/hour. In some embodiments, the regeneration buffer is passed through the ion exchange chromatography material at about 10 material volumes/hour.

In some embodiments of the invention, the polypeptide is produced in a host cell. In some embodiments, the host cell is a Chinese hamster ovary (CHO) cell. In some embodiments, the impurity comprises one or more of IgG fragments, host cell proteins, or host cell nucleic acids. In some embodiments, the polypeptide is produced in a CHO cell and the impurity comprises CHO host cell protein (CHOP) and/or CHO nucleic acids.

### BRIEF DESCRIPTION OF THE DRAWINGS

FIG. 1 is a chromatogram of an overload chromatography run without a regeneration phase. Fouling of the resin is observed during a pressure spike at the start of the sanitization phase.

FIG. 2 shows an example of a POROS™ 50HS chromatogram from overload chromatography of antibody mAb1.

FIG. 3 shows a chromatogram of size exclusion chromatography (SEC) of regeneration phases from two evaluation runs. The regeneration buffer for evaluation run 1 was 50 mM Tris, 85 mM NaOAc pH 8.0. The regeneration buffer for evaluation run 2 was 350 mM NaOAc pH 8.3.

FIG. 4 shows a chromatogram of Evaluation Run A for antibody mAb1.

FIG. 5 shows a chromatogram of Evaluation Run B for antibody mAb1.

FIG. 6 shows a chromatogram of Evaluation Run A for antibody mAb2.

FIG. 7 shows a chromatogram of Evaluation Run B for antibody mAb2.

FIGS. 8A and 8B show SEC-HPLC chromatograms of POROS™ 50HS mock-run cleaning procedures. In FIG. 8A the order of regeneration buffers is 1.) 50 mM Tris, 85 mM NaOAc pH 8.0 followed by 2.) 350 mM NaOAc pH 8.3. In FIG. 8B the order of regeneration buffers is 1.) 350 mM NaOAc pH 8.3 followed by 2.) 50 mM Tris, 85 mM NaOAc pH 8.0.

FIG. 9 shows robotic impurity map screening on Capto™ Adhere resin. Binding of mAb3, Chinese hamster ovary cell protein (CHOP) and high molecular weight (HMW) polypeptides is shown.

FIGS. 10A-10E show chromatograms of evaluation runs A to E, respectively, for mAb3.

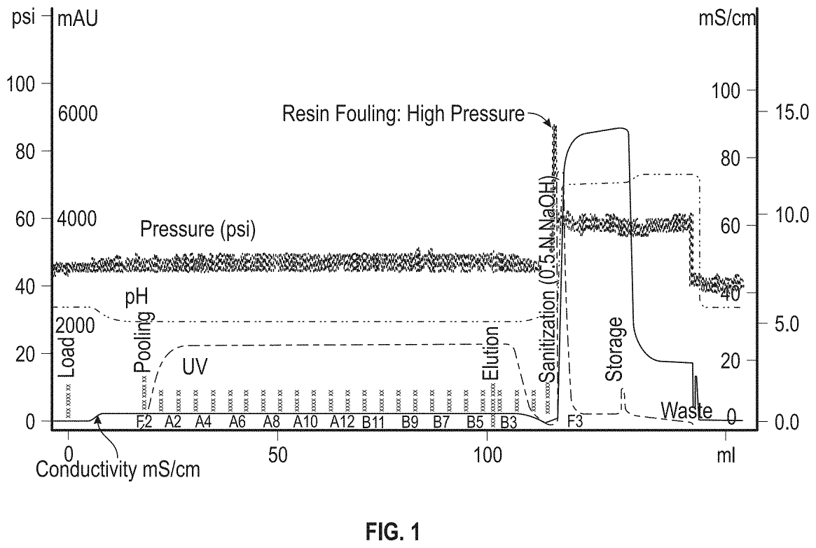

### DETAILED DESCRIPTION OF THE INVENTION

Provided herein are methods to clean an ion exchange chromatography material for reuse wherein the chromatography material is used in overload mode in the purification of a polypeptide, the method comprising the steps of a) passing a regeneration buffer through the ion exchange chromatography material, wherein the regeneration buffer comprises sodium acetate; b) passing of sanitization buffer through the ion exchange chromatography material; and c) storing the ion exchange chromatography material in a storage buffer. In some aspects, the inventions provides methods to clean an ion exchange chromatography material for reuse wherein the chromatography material is used in overload mode in the purification of a polypeptide, the method comprising the steps of a) passing a regeneration buffer through the ion exchange chromatography material, wherein the regeneration buffer comprises Tris and sodium acetate; b) passing a sanitization buffer through the ion exchange chromatography material, and c) storing the ion exchange chromatography material in a storage buffer.

Provided herein are methods to clean a cation exchange chromatography material for reuse wherein the chromatography material is used in overload mode in the purification of a polypeptide, the method comprising the steps of a) passing a regeneration buffer through the cation exchange chromatography material, wherein the regeneration buffer comprises sodium acetate; b) passing of sanitization buffer through the cation exchange chromatography material; and c) storing the cation exchange chromatography material in a storage buffer. In some aspects, the inventions provides methods to clean a cation exchange chromatography material for reuse wherein the chromatography material is used in overload mode in the purification of a polypeptide, the method comprising the steps of a) passing a regeneration buffer through the cation exchange chromatography material, wherein the regeneration buffer comprises Tris and sodium acetate; b) passing a sanitization buffer through the cation exchange chromatography material; and c) storing the cation exchange chromatography material in a storage buffer.

Provided herein are methods to clean an anion exchange chromatography material for reuse wherein the chromatography material is used in overload mode in the purification of a polypeptide, the method comprising the steps of a) passing a regeneration buffer through the anion exchange chromatography material, wherein the regeneration buffer comprises sodium acetate; b) passing of sanitization buffer through the anion exchange chromatography material; and c) storing the anion exchange chromatography material in a storage buffer. In some aspects, the inventions provides methods to clean a anion exchange chromatography material for reuse wherein the chromatography material is used in overload mode in the purification of a polypeptide, the method comprising the steps of a) passing a regeneration buffer through the anion exchange chromatography material, wherein the regeneration buffer comprises Tris and sodium acetate; b) passing a sanitization buffer through the anion exchange chromatography material; and c) storing the anion exchange chromatography material in a storage buffer.

In some aspects, the invention provides a method to clean an ion exchange chromatography material for reuse wherein the chromatography material is used in overload mode in the purification of a polypeptide, the method comprising the steps of a) passing a regeneration buffer through the ion exchange chromatography material, wherein the regeneration buffer comprises sodium acetate; b) passing an acid wash through the ion exchange chromatography material, c) passing of sanitization buffer through the ion exchange chromatography material, wherein the sanitization buffer comprises about 0.5 N NaOH and about 1 M NaCl; and d) storing the ion exchange chromatography material in a storage buffer, wherein the storage buffer comprises about 0.1 N NaOH. In some embodiments, the ion exchange material is an anion exchange material. In some embodiments, the ion exchange material is a mixed mode anion exchange material.

In some aspects, the invention provides methods for purifying a polypeptide from a composition comprising the polypeptide and an impurity using an ion exchange chromatography material wherein the ion exchange chromatography material is suitable for reuse, the method comprising the steps of a) equilibrating a ion exchange chromatography material with an equilibration buffer; b) loading the composition on the ion exchange chromatography material, wherein the load density is ≤about 1000 g/L; c) collecting fractions comprising the polypeptide; d) washing the ion exchange chromatography material with equilibration buffer; e) passing a regeneration buffer through the ion exchange chromatography material, wherein the regeneration buffer comprises sodium acetate; f) passing of sanitization buffer through the ion exchange chromatography material; and g) storing the ion exchange chromatography material in a storage buffer. In some aspects, the invention provides a method for purifying a polypeptide from a composition comprising the polypeptide and an impurity using an ion exchange chromatography material wherein the ion exchange chromatography material is suitable for reuse, the method comprising the steps of a) equilibrating ab ion exchange chromatography material with an equilibration buffer; b) loading the composition on the ion exchange chromatography material, wherein the load density is ≤about 1000 g/L; c) collecting fractions comprising the polypeptide; d) washing the ion exchange chromatography material with equilibration buffer; e) passing a regeneration buffer through the ion exchange chromatography material, wherein the regeneration buffer comprises Tris and sodium acetate; f) passing a sanitization buffer through the ion exchange chromatography material; g) storing the ion exchange chromatography material in a storage buffer.

In some aspects, the invention provides methods for purifying a polypeptide from a composition comprising the polypeptide and an impurity using a cation exchange chromatography material wherein the cation exchange chromatography material is suitable for reuse, the method comprising the steps of a) equilibrating a cation exchange chromatography material with an equilibration buffer; b) loading the composition on the cation exchange chromatography material, wherein the load density is ≤about 1000 g/L; c) collecting fractions comprising the polypeptide; d) washing the cation exchange chromatography material with equilibration buffer; e) passing a regeneration buffer through the cation exchange chromatography material, wherein the regeneration buffer comprises sodium acetate; f) passing of sanitization buffer through the cation exchange chromatography material; and g) storing the cation exchange chromatography material in a storage buffer. In some aspects, the invention provides a method for purifying a polypeptide from a composition comprising the polypeptide and an impurity using a cation exchange chromatography material wherein the cation exchange chromatography material is suitable for reuse, the method comprising the steps of a) equilibrating a cation exchange chromatography material with an equilibration buffer; b) loading the composition on the cation exchange chromatography material, wherein the load density is 5 about 1000 g/L; c) collecting fractions comprising the polypeptide; d) washing the cation exchange chromatography material with equilibration buffer; c) passing a regeneration buffer through the cation exchange chromatography material, wherein the regeneration buffer comprises Tris and sodium acetate; f) passing a sanitization buffer through the cation exchange chromatography material; g) storing the cation exchange chromatography material in a storage buffer.

In some aspects, the invention provides methods for purifying a polypeptide from a composition comprising the polypeptide and an impurity using an anion exchange chromatography material wherein the anion exchange chromatography material is suitable for reuse, the method comprising the steps of a) equilibrating a anion exchange chromatography material with an equilibration buffer; b) loading the composition on the anion exchange chromatography material, wherein the load density is ≤about 1000 g/L; c) collecting fractions comprising the polypeptide; d) washing the anion exchange chromatography material with equilibration buffer; e) passing a regeneration buffer through the anion exchange chromatography material, wherein the regeneration buffer comprises sodium acetate; f) passing of sanitization buffer through the anion exchange chromatography material; and g) storing the anion exchange chromatography material in a storage buffer. In some aspects, the invention provides a method for purifying a polypeptide from a composition comprising the polypeptide and an impurity using a anion exchange chromatography material wherein the anion exchange chromatography material is suitable for ruse, the method comprising the steps of a) equilibrating a anion exchange chromatography material with an equilibration buffer; b) loading the composition on the anion exchange chromatography material, wherein the load density is ≤about 1000 g/L; c) collecting fractions comprising the polypeptide; d) washing the anion exchange chromatography material with equilibration buffer; el passing a regeneration buffer through the anion exchange chromatography material, wherein the regeneration buffer comprises Tris and sodium acetate; f) passing a sanitization buffer through the anion exchange chromatography material; g) storing the anion exchange chromatography material in a storage buffer.

In some aspects, the invention provides methods for purifying a polypeptide from a composition comprising the polypeptide and an impurity using an ion exchange chromatography material wherein the ion exchange chromatography material is suitable for reuse, the method comprising the steps of a) equilibrating a ion exchange chromatography material with an equilibration buffer; b) loading the composition on the ion exchange chromatography material, wherein the load density is ≥20 g/L; c) collecting fractions comprising the polypeptide; d) washing the ion exchange chromatography material with equilibration buffer; e) passing a regeneration buffer through the ion exchange chromatography material, wherein the regeneration buffer comprises sodium acetate; f) passing of sanitization buffer through the ion exchange chromatography material; and g) storing the ion exchange chromatography material in a storage buffer. In some aspects, the invention provides a method for purifying a polypeptide from a composition comprising the polypeptide and an impurity using an ion exchange chromatography material wherein the ion exchange chromatography material is suitable for reuse, the method comprising the steps of a) equilibrating ab ion exchange chromatography material with an equilibration buffer; b) loading the composition on the ion exchange chromatography material, wherein the load density is ≥20 g/L; c) collecting fractions comprising the polypeptide; d) washing the ion exchange chromatography material with equilibration buffer; e) passing a regeneration buffer through the ion exchange chromatography material, wherein the regeneration buffer comprises Tris and sodium acetate; f) passing a sanitization buffer through the ion exchange chromatography material; g) storing the ion exchange chromatography material in a storage buffer.

In some aspects, the invention provides methods for purifying a polypeptide from a composition comprising the polypeptide and an impurity using a cation exchange chromatography material wherein the cation exchange chromatography material is suitable for reuse, the method comprising the steps of a) equilibrating a cation exchange chromatography material with an equilibration buffer; b) loading the composition on the cation exchange chromatography material, wherein the load density is ≥20 g/L; c) collecting fractions comprising the polypeptide; d) washing the cation exchange chromatography material with equilibration buffer; e) passing a regeneration buffer through the cation exchange chromatography material, wherein the regeneration buffer comprises sodium acetate; f) passing of sanitization buffer through the cation exchange chromatography material; and g) storing the cation exchange chromatography material in a storage buffer. In some aspects, the invention provides a method for purifying a polypeptide from a composition comprising the polypeptide and an impurity using a cation exchange chromatography material wherein the cation exchange chromatography material is suitable for reuse, the method comprising the steps of a) equilibrating a cation exchange chromatography material with an equilibration buffer; b) loading the composition on the cation exchange chromatography material, wherein the load density is ≥20 g/L; c) collecting fractions comprising the polypeptide; d) washing the cation exchange chromatography material with equilibration buffer; c) passing a regeneration buffer through the cation exchange chromatography material, wherein the regeneration buffer comprises Tris and sodium acetate; f) passing a sanitization buffer through the cation exchange chromatography material; g) storing the cation exchange chromatography material in a storage buffer.

In some aspects, the invention provides methods for purifying a polypeptide from a composition comprising the polypeptide and an impurity using an anion exchange chromatography material wherein the anion exchange chromatography material is suitable for reuse, the method comprising the steps of a) equilibrating a anion exchange chromatography material with an equilibration buffer; b) loading the composition on the anion exchange chromatography material, wherein the load density is ≥20 g/L; c) collecting fractions comprising the polypeptide; d) washing the anion exchange chromatography material with equilibration buffer; e) passing a regeneration buffer through the anion exchange chromatography material, wherein the regeneration buffer comprises sodium acetate; f) passing of sanitization buffer through the anion exchange chromatography material; and g) storing the anion exchange chromatography material in a storage buffer. In some aspects, the invention provides a method for purifying a polypeptide from a composition comprising the polypeptide and an impurity using a anion exchange chromatography material wherein the anion exchange chromatography material is suitable for reuse, the method comprising the steps of a) equilibrating a anion exchange chromatography material with an equilibration buffer; b) loading the composition on the anion exchange chromatography material, wherein the load density is ≥20 g/L; c) collecting fractions comprising the polypeptide; d) washing the anion exchange chromatography material with equilibration buffer; e) passing a regeneration buffer through the anion exchange chromatography material, wherein the regeneration buffer comprises Tris and sodium acetate; f) passing a sanitization buffer through the anion exchange chromatography material; g) storing the anion exchange chromatography material in a storage buffer.

In some aspects, the invention provides methods for purifying a polypeptide from a composition comprising the polypeptide and an impurity using an ion exchange chromatography material wherein the ion exchange chromatography material is suitable for reuse, the method comprising the steps of a) equilibrating a ion exchange chromatography material with an equilibration buffer; b) loading the composition on the ion exchange chromatography material, wherein the load density exceeds the dynamic binding capacity of the ion exchange chromatography material for the polypeptide; c) collecting fractions comprising the polypeptide; d) washing the ion exchange chromatography material with equilibration buffer; e) passing a regeneration buffer through the ion exchange chromatography material, wherein the regeneration buffer comprises sodium acetate; f) passing of sanitization buffer through the ion exchange chromatography material; and g) storing the ion exchange chromatography material in a storage buffer. In some aspects, the invention provides a method for purifying a polypeptide from a composition comprising the polypeptide and an impurity using an ion exchange chromatography material wherein the ion exchange chromatography material is suitable for reuse, the method comprising the steps of a) equilibrating ab ion exchange chromatography material with an equilibration buffer; b) loading the composition on the ion exchange chromatography material, wherein the load density exceeds the dynamic binding capacity of the ion exchange chromatography material for the polypeptide; c) collecting fractions comprising the polypeptide; d) washing the ion exchange chromatography material with equilibration buffer; e) passing a regeneration buffer through the ion exchange chromatography material, wherein the regeneration buffer comprises Tris and sodium acetate; f) passing a sanitization buffer through the ion exchange chromatography material; g) storing the ion exchange chromatography material in a storage buffer.

In some aspects, the invention provides methods for purifying a polypeptide from a composition comprising the polypeptide and an impurity using a cation exchange chromatography material wherein the cation exchange chromatography material is suitable for reuse, the method comprising the steps of a) equilibrating a cation exchange chromatography material with an equilibration buffer; b) loading the composition on the cation exchange chromatography material, wherein the load density exceeds the dynamic binding capacity of the cation exchange chromatography material for the polypeptide; c) collecting fractions comprising the polypeptide; d) washing the cation exchange chromatography material with equilibration buffer; e) passing a regeneration buffer through the cation exchange chromatography material, wherein the regeneration buffer comprises sodium acetate; f) passing of sanitization buffer through the cation exchange chromatography material; and g) storing the cation exchange chromatography material in a storage buffer. In some aspects, the invention provides a method for purifying a polypeptide from a composition comprising the polypeptide and an impurity using a cation exchange chromatography material wherein the cation exchange chromatography material is suitable for reuse, the method comprising the steps of a) equilibrating a cation exchange chromatography material with an equilibration buffer; b) loading the composition on the cation exchange chromatography material, wherein the load density exceeds the dynamic binding capacity of the cation exchange chromatography material for the polypeptide; c) collecting fractions comprising the polypeptide; d) washing the cation exchange chromatography material with equilibration buffer; e) passing a regeneration buffer through the cation exchange chromatography material, wherein the regeneration buffer comprises Tris and sodium acetate; f) passing a sanitization buffer through the cation exchange chromatography material; g) storing the cation exchange chromatography material in a storage buffer.

In some aspects, the invention provides methods for purifying a polypeptide from a composition comprising the polypeptide and an impurity using an anion exchange chromatography material wherein the anion exchange chromatography material is suitable for reuse, the method comprising the steps of a) equilibrating a anion exchange chromatography material with an equilibration buffer; b) loading the composition on the anion exchange chromatography material, wherein the load density exceeds the dynamic binding capacity of the anion exchange chromatography material for the polypeptide; c) collecting fractions comprising the polypeptide; d) washing the anion exchange chromatography material with equilibration buffer; e) passing a regeneration buffer through the anion exchange chromatography material, wherein the regeneration buffer comprises sodium acetate; f) passing of sanitization buffer through the anion exchange chromatography material; and g) storing the anion exchange chromatography material in a storage buffer. In some aspects, the invention provides a method for purifying a polypeptide from a composition comprising the polypeptide and an impurity using a anion exchange chromatography material wherein the anion exchange chromatography material is suitable for reuse, the method comprising the steps of a) equilibrating a anion exchange chromatography material with an equilibration buffer; b) loading the composition on the anion exchange chromatography material, wherein the load density exceeds the dynamic binding capacity of the anion exchange chromatography material for the polypeptide; c) collecting fractions comprising the polypeptide; d) washing the anion exchange chromatography material with equilibration buffer; e) passing a regeneration buffer through the anion exchange chromatography material, wherein the regeneration buffer comprises Tris and sodium acetate; f) passing a sanitization buffer through the anion exchange chromatography material; g) storing the anion exchange chromatography material in a storage buffer.

In some aspects, the invention provides a method for purifying a polypeptide from a composition comprising the polypeptide and an impurity using an ion exchange chromatography material wherein the ion exchange chromatography material is suitable for reuse, the method comprising the steps of a) equilibrating an ion exchange chromatography material with an equilibration buffer; b) loading the composition on the ion exchange chromatography material, wherein the load density exceeds the dynamic binding capacity of the ion exchange chromatography for the polypeptide; c) collecting fractions comprising the polypeptide; d) washing the ion exchange chromatography material with equilibration buffer; e) passing a regeneration buffer through the ion exchange chromatography material, wherein the regeneration buffer comprises sodium acetate; f) passing an acid wash through the ion exchange chromatography material, g) passing a sanitization buffer through the ion exchange chromatography material, wherein the sanitization buffer comprises about 0.5 N NaOH and about 1 M NaCl; and h) storing the ion exchange chromatography material in a storage buffer, wherein the storage buffer comprises about 0.1 N NaOH. In some embodiments, the ion exchange chromatography material is a cation exchange chromatography material. In some embodiments, the ion exchange chromatography material is an anion exchange chromatography material. In some embodiments, the ion exchange chromatography material is a mixed mode anion exchange chromatography material. In some embodiments, the ion exchange chromatography material is a Capto™ Adhere chromatography material.

In some embodiments of the invention, the ion exchange chromatography (e.g., the cation exchange chromatography) is used in the large-scale production of the polypeptide. In some embodiments, the polypeptide is an antibody; e.g., a monoclonal antibody.

### Definitions

The term “polypeptide” or “protein” are used interchangeably herein to refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. The terms “polypeptide” and “protein” as used herein specifically encompass antibodies.

“Purified” poly peptide (e.g., antibody or immunoadhesin) means that the polypeptide has been increased in purity, such that it exists in a form that is more pure than it exists in its natural environment and/or when initially synthesized and/or amplified under laboratory conditions. Purity is a relative term and does not necessarily mean absolute purity.

As used herein, “overload chromatography” refers to a chromatography procedure where the product of interest is loaded beyond the dynamic binding capacity of the chromatography material for the product. Impurities bind more strongly than the product resulting in a purified product eluent after protein breakthrough is achieved. In some examples, the product of interested is collected in fractions in the flow through after the dynamic binding capacity has been reached.

As used herein, the “material volume” of a chromatography material is the total volume of chromatography material used in a chromatography procedure. For example, when the chromatography material is in a column, the material volume is the total volume of chromatography material in the column. In this example, the “material volume” can also be referred to as the “column volume.”

A polypeptide “which binds” an antigen of interest, e.g. a tumor-associated polypeptide antigen target, is one that binds the antigen with sufficient affinity such that the polypeptide is useful as a diagnostic and/or therapeutic agent in targeting a cell or tissue expressing the antigen, and does not significantly cross-react with other polypeptides. In such embodiments, the extent of binding of the polypeptide to a “non-target” polypeptide will be less than about 10% of the binding of the polypeptide to its particular target polypeptide as determined by fluorescence activated cell sorting (FACS) analysis or radioimmunoprecipitation (RIA).

With regard to the binding of a polypeptide to a target molecule, the term “specific binding” or “specifically binds to” or is “specific for” a particular polypeptide or an epitope on a particular polypeptide target means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target, for example, an excess of non-labeled target. In this case, specific binding is indicated if the binding of the labeled target to a probe is competitively inhibited by excess unlabeled target.

The term “antibody” herein is used in the broadest sense and specifically covers monoclonal antibodies, polyclonal antibodies, multispecific antibodies (e.g. bispecific antibodies) formed from at least two intact antibodies, and antibody fragments so long as they exhibit the desired biological activity. The term “immunoglobulin” (Ig) is used interchangeable with antibody herein.

Antibodies are naturally occurring immunoglobulin molecules which have varying structures, all based upon the immunoglobulin fold. For example, IgG antibodies have two “heavy” chains and two “light” chains that are disulphide-bonded to form a functional antibody. Each heavy and light chain itself comprises a “constant” (C) and a “variable” (V) region. The V regions determine the antigen binding specificity of the antibody, whilst the C regions provide structural support and function in non-antigen-specific interactions with immune effectors. The antigen binding specificity of an antibody or antigen-binding fragment of an antibody is the ability of an antibody to specifically bind to a particular antigen.

The antigen binding specificity of an antibody is determined by the structural characteristics of the V region. The variability is not evenly distributed across the 110-amino acid span of the variable domains. Instead, the V regions consist of relatively invariant stretches called framework regions (FRs) of 15-30 amino acids separated by shorter regions of extreme variability called “hypervariable regions” that are each 9-12 amino acids long. The variable domains of native heavy and light chains each comprise four FRs, largely adopting a β-sheet configuration, connected by three hypervariable regions (“HVRs”), which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC).

Each V region typically comprises three hypervariable regions, for example, complementarity determining regions (“CDRs”), each of which contains a “hypervariable loop”, and four framework regions. An antibody binding site, the minimal structural unit required to bind with substantial affinity to a particular desired antigen, will therefore typically include the three CDRs, and at least three, preferably four, framework regions interspersed there between to hold and present the CDRs in the appropriate conformation. Classical four chain antibodies have antigen binding sites which are defined by V_{H} and V_{L} domains in cooperation. Certain antibodies, such as camel and shark antibodies, lack light chains and rely on binding sites formed by heavy chains only. Single domain engineered immunoglobulins can be prepared in which the binding sites are formed by heavy chains or light chains alone, in absence of cooperation between V_{H} and V_{L}.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions both in the light chain and the heavy chain variable domains. The more highly conserved portions of variable domains are called the framework regions (FRs). The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service. National Institutes of Health, Bethesda, Md. (1991)) The constant domains are not involved directly in binding an antibody to an antigen, but exhibit various effector functions, such as participation of the antibody in antibody dependent cellular cytotoxicity (ADCC).

The term “hypervariable region” when used herein refers to the amino acid residues of an antibody that are responsible for antigen binding. The hypervariable region may comprise amino acid residues from a “complementarity determining region” or “CDR” (e.g., around about residues 24-34 (L1), 50-56 (L2) and 89-97 (L3) in the V_{L}, and around about 31-35B (H1), 50-65 (H2) and 95-102 (H3) in the V_{H} (Kabat et al., Sequences of Proteins of Immunological Interest, 5th Ed. Public Health Service, National Institutes of Health, Bethesda, Md. (1991)) and/or those residues from a “hypervariable loop” (e.g. residues 26-32 (L1), 50-52 (L2) and 91-96 (L3) in the V_{L}, and 26-32 (H1), 52A-55 (H2) and 96-101 (H3) in the V_{H} (Chothia and Lesk J. Mol. Biol. 196:901-917 (1987)).

“Framework” or “FR” residues are those variable domain residues other than the hypervariable region residues as herein defined.

“Antibody fragments” comprise a portion of an intact antibody, preferably comprising the antigen binding region thereof. Examples of antibody fragments include Fab, Fab′, F(ab′)₂, and Fv fragments; diabodies; tandem diabodies (taDb), triabody, linear antibodies (e.g., U.S. Pat. No. 5,641,870, Example 2; Zapata et al., Protein Eng. 8(10):1057-1062 (1995)); one-armed antibodies, single variable domain antibodies, minibodies, single-chain antibody molecules; multispecific antibodies formed from antibody fragments (e.g., including but not limited to, db-Fc, taDb-Fc, taDb-CH3, (scFV)4-Fc, sc-Fv, di-scFv, bi-scFv, or tandem (di,tri)-scFv); Bi-specific T-cell engagers (BiTEs), and trifunctional antibody.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-binding sites and is still capable of cross-linking antigen.

“Fv” is the minimum antibody fragment that contains a complete antigen-recognition and antigen-binding site. This region consists of a dimer of one heavy chain and one light chain variable domain in tight, non-covalent association. It is in this configuration that the three hypervariable regions of each variable domain interact to define an antigen-binding site on the surface of the V_{H}-V_{L} dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site.

The Fab fragment also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear at least one free thiol group. F(ab′)₂ antibody fragments originally were produced as pairs of Fab′ fragments that have hinge cysteines between them. Other chemical couplings of antibody fragments are also known.

The “light chains” of antibodies (immunoglobulins) from any vertebrate species can be assigned to one of two clearly distinct types, called kappa (κ) and lambda (λ), based on the amino acid sequences of their constant domains.

Depending on the amino acid sequence of the constant domain of their heavy chains, antibodies can be assigned to different classes. There are five major classes of intact antibodies: IgA, IgD, IgE, IgG, and IgM, and several of these may be further divided into subclasses (isotypes), e.g., IgG1, IgG2, IgG3, IgG4, IgA, and IgA2. The heavy chain constant domains that correspond to the different classes of antibodies are called α, δ, ε, γ, and μ, respectively. The subunit structures and three-dimensional configurations of different classes of immunoglobulins are well known.

“Single-chain Fv” or “scFv” antibody fragments comprise the V_{H} and V_{L} domains of antibody, wherein these domains are present in a single polypeptide chain. In some embodiments, the Fv polypeptide further comprises a polypeptide linker between the V_{H} and V_{L} domains that enables the scFv to form the desired structure for antigen binding. For a review of scFv see Plückthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy chain variable domain (V_{H}) connected to a light chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097: WO 93/11161; and Hollinger et al., Proc. Natl. Acad. Sci. USA, 90:6444-6448 (1993).

The term “multispecific antibody” is used in the broadest sense and specifically covers an antibody that has polyepitopic specificity. Such multispecific antibodies include, but are not limited to, an antibody comprising a heavy chain variable domain (V_{H}) and a light chain variable domain (V_{L}), where the V_{H}V_{L} unit has polyepitopic specificity, antibodies having two or more V_{L} and V_{H} domains with each V_{H}V_{L} unit binding to a different epitope, antibodies having two or more single variable domains with each single variable domain binding to a different epitope, full length antibodies, antibody fragments such as Fab, Fv, dsFv, scFv, diabodies, bispecific diabodies, triabodies, tri-functional antibodies, antibody fragments that have been linked covalently or non-covalently. “Polyepitopic specificity” refers to the ability to specifically bind to two or more different epitopes on the same or different target(s). “Monospecific” refers to the ability to bind only one epitope. According to one embodiment, the multispecific antibody is an IgG antibody that binds to each epitope with an affinity of 5 μM to 0.001 pM, 3 μM to 0.001 pM, 1 μM to 0.001 pM, 0.5 μM to 0.001 pM, or 0.1 μM to 0.001 pM.

The expression “single domain antibodies” (sdAbs) or “single variable domain (SVD) antibodies” generally refers to antibodies in which a single variable domain (VH or VL) can confer antigen binding. In other words, the single variable domain does not need to interact with another variable domain in order to recognize the target antigen. Examples of single domain antibodies include those derived from camelids (lamas and camels) and cartilaginous fish (e.g., nurse sharks) and those derived from recombinant methods from humans and mouse antibodies (Nature (1989) 341:544-546; Dev Comp Immunol (2006) 30:43-56; Trend Biochem Sci (2001) 26:230-235; Trends Biotechnol (2003):21:484-490; WO 2005/035572; WO 03/035694; FEBs Lett (1994) 339:285-290; WO00/29004; WO 02/051870).

The term “monoclonal antibody” as used herein refers to an antibody obtained from a population of substantially homogeneous antibodies, i.e., the individual antibodies comprising the population are identical and/or bind the same epitope, except for possible variants that may arise during production of the monoclonal antibody, such variants generally being present in minor amounts. In contrast to polyclonal antibody preparations that typically include different antibodies directed against different determinants (epitopes), each monoclonal antibody is directed against a single determinant on the antigen. In addition to their specificity, the monoclonal antibodies are advantageous in that they are uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the methods provided herein may be made by the hybridoma method first described by Kohler et al., Nature 256:495 (1975), or may be made by recombinant DNA methods (see, e.g., U.S. Pat. No. 4,816,567). The “monoclonal antibodies” may also be isolated from phage antibody libraries using the techniques described in Clackson el al., Nature 352:624-628 (1991) and Marks et al., J. Mol. Bol. 222:581-597 (1991), for example.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA 81:6851-6855 (1984)). Chimeric antibodies of interest herein include “primatized” antibodies comprising variable domain antigen-binding sequences derived from a non-human primate (e.g. Old World Monkey, such as baboon, rhesus or cynomolgus monkey) and human constant region sequences (U.S. Pat. No. 5,693,780).

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence, except for FR substitution(s) as noted above. The humanized antibody optionally also will comprise at least a portion of an immunoglobulin constant region, typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

For the purposes herein, an “intact antibody” is one comprising heavy and light variable domains as well as an Fc region. The constant domains may be native sequence constant domains (e.g. human native sequence constant domains) or amino acid sequence variant thereof. Preferably, the intact antibody has one or more effector functions.

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain Particular amino acid residues are believed to form an interface between the light chain and heavy chain variable domains.

A “naked antibody” is an antibody (as herein defined) that is not conjugated to a heterologous molecule, such as a cytotoxic moiety or radiolabel.

In some embodiments, antibody “effector functions” refer to those biological activities attributable to the Fc region (a native sequence Fc region or amino acid sequence variant Fc region) of an antibody, and vary with the antibody isotype. Examples of antibody effector functions include C1q binding and complement dependent cytotoxicity; Fc receptor binding; antibody-dependent cell-mediated cytotoxicity (ADCC); phagocytosis; down regulation of cell surface receptors.

“Antibody-dependent cell-mediated cytotoxicity” and “ADCC” refer to a cell-mediated reaction in which nonspecific cytotoxic cells that express Fc receptors (FcRs) (e.g. Natural Killer (NK) cells, neutrophils, and macrophages) recognize bound antibody on a target cell and subsequently cause lysis of the target cell. The primary cells for mediating ADCC, NK cells, express FcγRIII only, whereas monocytes express FcγRI, FcγRII and FcγRIII. FcR expression on hematopoietic cells in summarized is Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al., Proc. Natl. Acad. Sci. (USA) 95:652-656 (1998).

“Human effector cells” are leukocytes that express one or more FcRs and perform effector functions. In some embodiments, the cells express at least FcγRIII and carry out ADCC effector function. Examples of human leukocytes that mediate ADCC include peripheral blood mononuclear cells (PBMC), natural killer (NK) cells, monocytes, cytotoxic T cells and neutrophils; with PBMCs and NK cells being preferred.

The terms “Fc receptor” or “FcR” are used to describe a receptor that binds to the Fc region of an antibody. In some embodiments, the FcR is a native sequence human FcR. Moreover, a preferred FcR is one that binds an IgG antibody (a gamma receptor) and includes receptors of the FcγRI, FcγRIII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (see Daëron, Annu. Rev. Immunol. 15:203-234 (1997)). FcRs are reviewed in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976) and Kim et al., J. Immunol. 24:249 (1994)).

The term “sequential” as used herein with regard to chromatography refers to having a first chromatography followed by a second chromatography. Additional steps may be included between the first chromatography and the second chromatography.

The term “continuous” as used herein with regard to chromatography refers to having a first chromatography material and a second chromatography material either directly connected or some other mechanism which allows for continuous flow between the two chromatography materials.

The term “isolated” as used herein refers to a molecule that has been separated from at least some of the components with which it is typically found in nature or produced. For example, a polypeptide is referred to as “isolated” when it is separated from at least some of the components of the cell in which it was produced. Where a polypeptide is secreted by a cell after expression, physically separating the supernatant containing the polypeptide from the cell that produced it is considered to be “isolating” the polypeptide. Similarly, a polynucleotide is referred to as “isolated” when it is not part of the larger polynucleotide (such as, for example, genomic DNA or mitochondrial DNA, in the case of a DNA polynucleotide) in which it is typically found in nature, or is separated from at least some of the components of the cell in which it was produced. e.g., in the case of an RNA polynucleotide. Thus, a DNA polynucleotide that is contained in a vector inside a host cell may be referred to as “isolated”.

“Contaminants” refer to materials that are different from the desired polypeptide product. The contaminant includes, without limitation: host cell materials, such as CHO host cell protein (CHOP); leached Protein A; nucleic acid, a variant, fragment, aggregate or derivative of the desired polypeptide; another polypeptide; endotoxin; viral contaminant; cell culture media component, etc. In some examples, the contaminant may be a host cell protein (HCP) from, for example but not limited to, a bacterial cell such as an E. coli cell, an insect cell, a prokaryotic cell, a eukaryotic cell, a yeast cell, a mammalian cell, an avian cell, a fungal cell.

Reference to “about” a value or parameter herein includes (and describes) variations that are directed to that value or parameter per se. For example, description referring to “about X” includes description of “X”.

As used herein and in the appended claims, the singular forms “a,” “or,” and “the” include plural referents unless the context clearly dictates otherwise. It is understood that aspects and variations of the invention described herein include “consisting” and/or “consisting essentially of” aspects and variations.

### Methods of Chromatography Cleaning

### Chromatography

The invention provides methods to clean or regenerate ion exchange chromatography materials for reuse wherein the ion exchange chromatography is used in an overload mode. In some embodiments, the chromatography materials are used for large-scale purification of a polypeptide; e.g., in manufacturing-scale production of polypeptides such as antibodies or fragments thereof. In some embodiments, the ion exchange chromatography material is a cation exchange chromatography material. In some embodiments, the ion exchange chromatography material is an anion exchange chromatography material.

In some aspects, the invention provides methods to clean an ion exchange chromatography material for reuse wherein the chromatography material is used in overload mode in the purification of a polypeptide, the method comprising the steps of a) passing a regeneration buffer through the ion exchange chromatography material, wherein the regeneration buffer comprises sodium acetate (NaOAc); b) passing of sanitization buffer through the ion exchange chromatography material; and c) storing the ion exchange chromatography material in a storage buffer. In some embodiments, the ion exchange chromatography material is a cation exchange chromatography material. In some embodiments, the ion exchange chromatography material is an anion exchange chromatography material.

In some embodiments, the regeneration buffer comprises about 50 to about 600 mM NaOAc. In some embodiments, the regeneration buffer comprises NaOAc at a concentration range of about any of 50 mM to 600 mM, 100 mM to 600 mM, 150 mM to 600 mM, 200 mM to 600 mM, 250 mM to 600 mM, 300 mM to 600 mM, 350 mM to 600 mM, 400 mM to 600 mM, 450 mM to 600 mM, 500 mM to 600 mM, 550 mM to 600 mM, 50 mM to 550 mM, 50 mM to 450 mM, 50 mM to 400 mM, 50 mM to 350 mM, 50 mM to 300 mM, 50 mM to 250 mM, 50 mM to 200 mM, 50 mM to 150 mM, 50 mM to 100 mM, 100 mM to 550 mM, 150 mM to 500 mM, 200 mM to 450 mM, 250 mM to 400 mM, or 300 mM to 400 mM. In some embodiments, the regeneration buffer comprises about any of 50, 100, 150, 200, 250, 300, 350, 400, 450, 500, 550, 600, or 650 mM NaOAc. In some embodiments, the regeneration buffer comprises about 350 mM NaOAc.

In some embodiments, the regeneration buffer comprises NaOAc at a concentration range of about any of 70 mM to 100 mM, 75 mM to 95 mM, or 80 mM to 90 mM. In some embodiments, the regeneration buffer comprises 85 mM NaOAc. In some embodiments, the regeneration buffer comprises NaOAc at a concentration range of about any of 70 mM to 100 mM, 75 mM to 95 mM, or 80 mM to 90 mM and further comprises Tris. In some embodiments, the regeneration buffer comprises NaOAc at a concentration range of about any of 70 mM to 100 mM, 75 mM to 95 mM, or 80 mM to 90 mM and further comprises about any of 25 mM to 75 mM, 30 mM to 75 mM, 35 mM to 40 mM, 45 mM to 75 mM, 50 mM to 75 mM, 25 mM to 70 mM, 25 mM to 65 mM, 25 mM to 60 mM, 25 mM to 55 mM, or 25 mM to 50 mM Tris. In some embodiments, the regeneration buffer comprises NaOAc at a concentration range of about any of 70 mM to 100 mM, 75 mM to 95 mM, or 80 mM to 90 mM and further comprises about any of 25 mM, 30 mM, 35 mM, 40 mM, 45 mM, 50 mM, 55 mM, 60 mM, 65 mM, 70 mM or 75 mM Tris. In some embodiments, the pH of the regeneration buffer is about pH 4.0 to about pH 11.0. In some embodiments, the pH of the regeneration buffer is about pH 4.0 to about pH 10.5, about pH 4.0 to about pH 10.0, about pH 4.0 to about pH 9.5, about pH 4.0 to about pH 9.0, about pH 4.0 to about pH 8.5, about pH 4.0 to about pH 8.0, about pH 5.5 to about pH 11.0, about pH 5.5 to about pH 10.5, about pH 5.5 to about pH 10.0, about pH 5.5 to about pH 9.5, about pH 5.5 to about pH 9.0, about pH 5.5 to about pH 8.5, about pH 5.5 to about pH 8.0, about pH 6.0 to about pH 11.0, about pH 6.5 to about pH 11.0, about pH 7.0 to about pH 11.0, about pH 7.5 to about pH 11.0, about pH 8.0 to about pH 11.0, about pH 6.0 to about pH 11.0, about pH 6.5 to about pH 10.5, about pH 7.0 to about pH 10.0, about pH 7.5 to about pH 9.5, or about pH 8.0 to about pH 9.0. In some embodiments, the regeneration buffer is about pH 7.5, about pH 8.0, about pH 8.1, about pH 8.2, about pH 8.3, about pH 8.4, about pH 8.5, or about pH 9.0. In some embodiments, the pH of the regeneration buffer is about pH 8.3. In some embodiments, the pH of the regeneration buffer comprises about 350 mM NaOAc at about pH 8.3. In some embodiments, the pH of the regeneration buffer is about pH 8. In some embodiments, the pH of the regeneration buffer comprises about 50 mM Tris and about 85 mM NaOAc at about pH 8.0.

In some embodiments, about 3 to about 12, about 3 to about 11, about 3 to about 10, about 3 to about 9, about 3 to about 8, about 3 to about 7, about 3 to about 6, about 3 to about 5, about 3 to about 4, about 4 to about 12, about 5 to about 12, about 6 to about 12, about 7 to about 12, about 8 to about 12, about 9 to about 12, about 10 to about 12, about 11 to about 12, about 5 to about 12, about 5 to about 10, about 8 to about 12, or about 9 to about 11 material volumes (e.g., column volumes) of regeneration buffer are passed through the ion exchange chromatography material. In some embodiments, about any of 3, 4, 5, 6, 7, 8, 9, 10, 11, or 12 material volumes (e.g., column volumes) of regeneration buffer are passed through the ion exchange chromatography material. In some embodiments, about 10 material volumes (e.g., column volumes) of regeneration buffer are passed through the ion exchange chromatography material.

In some aspects, the invention provides a method to clean an ion exchange chromatography material for reuse wherein the chromatography material is used in overload mode in the purification of a polypeptide, the method comprising the steps of a) passing a regeneration buffer through the ion exchange chromatography material, wherein the regeneration buffer comprises sodium acetate; b) passing an acid wash through the ion exchange chromatography material, c) passing of sanitization buffer through the ion exchange chromatography material, wherein the sanitization buffer comprises about 0.5 N NaOH and about 1 M NaCl; and d) storing the ion exchange chromatography material in a storage buffer, wherein the storage buffer comprises about 0.1 N NaOH. In some embodiments, the ion exchange chromatography material is a cation exchange chromatography material. In some embodiments, the ion exchange chromatography material is an anion exchange chromatography material. In some embodiments, the ion exchange chromatography material is a mixed mode anion exchange chromatography material. In some embodiments, the ion exchange chromatography material is a Capto™ Adhere chromatography material.

In some embodiments, the regeneration buffer comprises about 50 to about 600 mM NaOAc. In some embodiments, the regeneration buffer comprises NaOAc at a concentration range of about any of 50 mM to 600 mM, 100 mM to 600 mM, 150 mM to 600 mM, 200 mM to 600 mM, 250 mM to 600 mM, 300 mM to 600 mM, 350 mM to 600 mM, 400 mM to 600 mM, 450 mM to 600 mM, 500 mM to 600 mM, 550 mM to 600 mM, 50 mM to 550 mM, 50 mM to 450 mM, 50 mM to 400 mM, 50 mM to 350 mM, 50 mM to 300 mM, 50 mM to 250 mM, 50 mM to 200 mM, 50 mM to 150 mM, 50 mM to 100 mM, 100 mM to 550 mM, 150 mM to 500 mM, 200 mM to 450 mM, 250 mM to 400 mM, or 300 mM to 400 mM. In some embodiments, the regeneration buffer comprises about any of 50, 100, 150, 200, 250, 300, 350, 400, 450, 500, 550, 600, or 650 mM NaOAc. In some embodiments, the regeneration buffer comprises about 350 mM NaOAc.

In some embodiments, the pH of the regeneration buffer is about pH 2.0 to about pH 7.0. In some embodiments, the pH of the regeneration buffer is about pH 2.0 to about pH 6.5, about pH 2.0 to about pH 6.0, about pH 2.0 to about pH 5.5, about pH 2.0 to about pH 5.0, about pH 2.0 to about pH 4.5, about pH 2.0 to about pH 4.0, about pH 2.0 to about pH13.5, about pH 2.0 to about pH 3.0, about pH 2.0 to about pH 2.5, about pH 2.5 to about pH 7.0, about pH 3.0 to about pH 7.0, about pH 3.5 to about pH 7.0, about pH 4.0 to about pH 7.0, about pH 4.5 to about pH 7.0, about pH 5.0 to about pH 7.0, about pH 5.5 to about pH 7.0, about pH 6.0 to about pH 7.0, or about pH 6.5 to about pH 7.0. In some embodiments, the regeneration buffer is about pH 2.0, about pH 8.0, about pH 2.5, about pH 3.0, about pH 3.5, about pH 4.0, about pH 4.5, about pH 5.0, about pH 5.5, about pH 6.0, about pH 6.5, or about pH 7.0. In some embodiments, the regeneration buffer is 350 mM NaOAc, pH 4.0. In some embodiments, the regeneration buffer is 350 mM NaOAc, pH 5.0. In some embodiments, the regeneration buffer is 40 mM NaOAc, pH 4.0. In some embodiments, the regeneration buffer is 40 mM NaOAc, pH 5.0.

In some embodiments, about 3 to about 12, about 3 to about 11, about 3 to about 10, about 3 to about 9, about 3 to about 8, about 3 to about 7, about 3 to about 6, about 3 to about 5, about 3 to about 4, about 4 to about 12, about 5 to about 12, about 6 to about 12, about 7 to about 12, about 8 to about 12, about 9 to about 12, about 10 to about 12, about 11 to about 12, about 5 to about 12, about 5 to about 10, about 8 to about 12, or about 9 to about 11 material volumes (e.g., column volumes) of regeneration buffer are passed through the ion exchange chromatography material. In some embodiments, about any of 3, 4, 5, 6, 7, 8, 9, 10, 1, or 12 material volumes (e.g., column volumes) of regeneration buffer are passed through the ion exchange chromatography material. In some embodiments, about 7 material volumes (e.g., column volumes) of regeneration buffer are passed through the ion exchange chromatography material.

In some embodiments, the acid wash comprises NaOAc. In some embodiments, the acid wash comprises phosphoric acid. In some embodiments, the acid wash comprises NaOAc and phosphoric acid. In some embodiments, the concentration of NaOAc in the acid wash is more than about any of 0.1M to less than about 0.5 M. In some embodiments, the concentration of NaOAc in the acid wash is any of about 0.100 M, 0.120M, 0.130M, 0.140M, 0.150M, 0.160M, 0.161M, 0.162M, 0.163M, 0.164M, 0.167M, 0.168M, 0.169M, 0.170M, 0.180M, 0.190M, 0.200M, 0.250M, 0.300M, 0.350M, 0.400M, 0.450M, or 0.500M. In some embodiments, the concentration of phosphoric acid in the acid wash is any of about 0.10 M, 0.15M, 0.20M, 0.25M, 0.30M, 0.35M, 0.40M, 0.45M, 0.50M, 0.55M, or 0.60M. In some embodiments, the acid wash comprises about 0.167M NaOAc and about 0.3M phosphoric acid.

In some embodiments, about 1 to about 5, about 1 to about 4, about 1 to about 3, about 1 to about 2, about 2 to about 5, about 2 to about 4, about 2 to about 3, about 3 to about 5, about 3 to about 4, about 4 to about 5 material volumes (e.g., column volumes) of acid wash are passed through the ion exchange chromatography material. In some embodiments, about any of 1, 2, 3, 4, or 5 material volumes (e.g., column volumes) of acid wash are passed through the ion exchange chromatography material. In some embodiments, about 3 material volumes (e.g., column volumes) of acid wash are passed through the ion exchange chromatography material.

In some aspects, the invention provides methods to clean an ion exchange chromatography material for reuse wherein the chromatography material is used in overload mode in the purification of a polypeptide, the method comprising the steps of a) passing a first regeneration buffer through the ion exchange chromatography material, wherein the regeneration buffer comprises sodium acetate; b) passing a second regeneration buffer through the ion exchange chromatography material, wherein the second regeneration buffer comprises sodium acetate c) passing an acid wash through the ion exchange chromatography material, passing of sanitization buffer through the ion exchange chromatography material, wherein the sanitization buffer comprises about 0.5 N NaOH and about 1 M NaCl; and d) storing the ion exchange chromatography material in a storage buffer, wherein the storage buffer comprises about 0.1 N NaOH. In some embodiments, the ion exchange chromatography material is a cation exchange chromatography material. In some embodiments, the ion exchange chromatography material is an anion exchange chromatography material. In some embodiments, the ion exchange chromatography material is a mixed mode anion exchange chromatography material. In some embodiments, the ion exchange chromatography material is a Capto™ Adhere chromatography material.

In some embodiments, the first regeneration buffer and/or the second regeneration buffer comprises about 50 to about 600 mM NaOAc. In some embodiments, the first regeneration buffer and/or the second regeneration buffer comprises NaOAc at a concentration range of about any of 25 mM to 600 mM, 30 mM to 600 mM, 40 mM to 600 mM, 50 mM to 600 mM, 75 mM to 600 mM, 100 mM to 600 mM, 125 mM to 600 mM, 150 mM to 600 mM, 175 mM to 600 mM, 200 mM to 600 mM, 225 mM to 600 mM, 250 mM to 600 mM, 275 mM to 600 mM, 300 mM to 600 mM, 325 mM to 600 mM, 350 mM to 600 mM, 375 mM to 600 mM, 400 mM to 600 mM, 450 mM to 600 mM, 500 mM to 600 mM, 550 mM to 600 mM, 25 mM to 550 mM, 25 mM to 500 mM, 25 mM to 475 mM, 25 mM to 450 mM, 25 mM to 425 mM, 25 mM to 400 mM, 25 mM to 375 mM, 25 mM to 550 mM, 25 mM to 325 mM, 25 mM to 300 mM, 25 mM to 275 mM, 25 mM to 250 mM, 25 mM to 225 mM, 25 mM to 200 mM, 25 mM to 175 mM, 25 mM to 150 mM, 25 mM to 125 mM, 25 mM to 100 mM, 25 mM to 90 mM, 25 mM to 80 mM, 25 mM to 70 mM, 25 mM to 60 mM, 25 mM to 50 mM, 25 mM to 40 mM, or 25 mM to 30 mM. In some embodiments, the first regeneration buffer comprises about any of 10, 20, 30, 40, 50, 60, 70, 80, 90, 100, 150, 200, 250, 300, 350, 400, 450, 500, 550, or 600 mM NaOAc. In some embodiments, the regeneration buffer comprises about 350 mM NaOAc.

In some embodiments, the pH of the first regeneration buffer and/or the second regeneration buffer is about pH 2.0 to about pH 7.0. In some embodiments, the pH of the first regeneration buffer and/or the second regeneration buffer is about pH 2.0 to about pH 6.5, about pH 2.0 to about pH 6.0, about pH 2.0 to about pH 5.5, about pH 2.0 to about pH 5.0, about pH 2.0 to about pH 4.5, about pH 2.0 to about pH 4.0, about pH 2.0 to about pH 3.5, about pH 2.0 to about pH 3.0, about pH 2.0 to about pH 2.5, about pH 2.5 to about pH 7.0, about pH 3.0 to about pH 7.0, about pH 3.5 to about pH 7.0, about pH 4.0 to about pH 7.0, about pH 4.5 to about pH 7.0, about pH 5.0 to about pH 7.0, about pH 5.5 to about pH 7.0, about pH 6.0 to about pH 7.0, or about pH 6.5 to about pH 7.0. In some embodiments, the regeneration buffer is about pH 2.0, about pH 8.0, about pH 2.5, about pH 3.0, about pH 3.5, about pH 4.0, about pH 4.5, about pH 5.0, about pH 5.5, about pH 6.0, about pH 6.5, or about pH 7.0.

In some embodiments, the first regeneration buffer is about 40 mM NaOAc and about pH 4.0, and the second regeneration buffer is about 350 mM NaOAc and about pH 4.0. In some embodiments, the first regeneration buffer is about 40 mM NaOAc and about pH 5.0, and the second regeneration buffer is about 350 mM NaOAc and about pH 5.0. In some embodiments, the first regeneration buffer is about 350 mM NaOAc and about pH 4.0, and the second regeneration buffer is about 40 mM NaOAc and about pH 4.0. In some embodiments, the first regeneration buffer is about 350 mM NaOAc and about pH 5.0, and the second regeneration buffer is about 40 mM NaOAc and about pH 5.0.

In some embodiments, about 3 to about 12, about 3 to about 11, about 3 to about 10, about 3 to about 9, about 3 to about 8, about 3 to about 7, about 3 to about 6, about 3 to about 5, about 3 to about 4, about 4 to about 12, about 5 to about 12, about 6 to about 12, about 7 to about 12, about 8 to about 12, about 9 to about 12, about 10 to about 12, about 11 to about 12, about 5 to about 12, about 5 to about 10, about 8 to about 12, or about 9 to about 11 material volumes (e.g., column volumes) of the first regeneration buffer and/or the second regeneration buffer are passed through the ion exchange chromatography material. In some embodiments, about any of 3, 4, 5, 6, 7, 8, 9, 10, 11, or 12 material volumes (e.g., column volumes) of the first regeneration buffer and/or the second regeneration buffer are passed through the ion exchange chromatography material. In some embodiments, about 10 material volumes (e.g., column volumes) of first regeneration buffer and/or the second regeneration buffer are passed through the ion exchange chromatography material.

In some embodiments, the acid wash comprises NaOAc. In some embodiments, the acid wash comprises phosphoric acid. In some embodiments, the acid wash comprises NaOAc and phosphoric acid. In some embodiments, the concentration of NaOAc in the acid wash is more than about any of 0.1M to less than about 0.5 M. In some embodiments, the concentration of NaOAc in the acid wash is any of about 0.100 M, 0.120M, 0.130M, 0.140M, 0.150M, 0.160M, 0.161M, 0.162M, 0.163M, 0.164M, 0.167M, 0.168M, 0.169M, 0.170M, 0.180M, 0.190M, 0.200M, 0.250M, 0.300M, 0.350M, 0.400M, 0.450M, or 0.500M. In some embodiments, the concentration of phosphoric acid in the acid wash is any of about 0.10 M, 0.15M, 0.20M, 0.25M, 0.30M, 0.35M, 0.40M, 0.45M, 0.50M, 0.55M, or 0.60M. In some embodiments, the acid wash comprises about 0.167M NaOAc and about 0.3M phosphoric acid.

In some embodiments, about 1 to about 5, about 1 to about 4, about 1 to about 3, about 1 to about 2, about 2 to about 5, about 2 to about 4, about 2 to about 3, about 3 to about 5, about 3 to about 4, about 4 to about 5 material volumes (e.g., column volumes) of acid wash are passed through the ion exchange chromatography material. In some embodiments, about any of 1, 2, 3, 4, or 5 material volumes (e.g., column volumes) of acid wash are passed through the ion exchange chromatography material. In some embodiments, about 3 material volumes (e.g., column volumes) of acid wash are passed through the ion exchange chromatography material.

In some embodiments, the sanitization buffer comprises NaOH. In some embodiments, the sanitization buffer comprises about 0.1 N to about 1.0 N NaOH, about 0.1 N to about 0.9 N NaOH, about 0.1 N to about 0.8 N NaOH, about 0.1 N to about 0.7 N NaOH, about 0.1 N to about 0.8 N NaOH, about 0.1 N to about 0.6 N NaOH, about 0.1 N to about 0.5 N NaOH, about 0.2 N to about 1.0 N NaOH, about 0.3 N to about 1.0 N NaOH, about 0.4 N to about 1.0 N NaOH, about 0.5 N to about 1.0 N NaOH, about 0.2 N to about 0.8 N NaOH, about 0.3 N to about 0.7 N NaOH, or about 0.4 N to about 0.6 N NaOH. In some embodiments, sanitization buffer comprises about any of 0.1 N, 0.2 N, 0.3 N, 0.4 N, 0.5 N, 0.6 N, 0.7 N NaOH, 0.8 N, 0.9 N, or 1.0 N NaOH. In some embodiments, sanitization buffer comprises about 0.5 N NaOH.

In some embodiments, the sanitization buffer comprises NaOH and NaCl. In some embodiments, the sanitization buffer comprises about 0.1 N to about 1.0 N NaOH, about 0.1 N to about 0.9 N NaOH, about 0.1 N to about 0.8 N NaOH, about 0.1 N to about 0.7 N NaOH, about 0.1 N to about 0.8 N NaOH, about 0.1 N to about 0.6 N NaOH, about 0.1 N to about 0.5 N NaOH, about 0.2 N to about 1.0 N NaOH, about 0.3 N to about 1.0 N NaOH, about 0.4 N to about 1.0 N NaOH, about 0.5 N to about 1.0 N NaOH, about 0.2 N to about 0.8 N NaOH, about 0.3 N to about 0.7 N NaOH, or about 0.4 N to about 0.6 N NaOH. In some embodiments, sanitization buffer comprises about any of 0.1 N, 0.2 N, 0.3 N, 0.4 N, 0.5 N, 0.6 N, 0.7 N NaOH, 0.8 N, 0.9 N, or 1.0 N NaOH. In some embodiments, the sanitization buffer comprises about 0.5 M to about 1.5 M NaCl, 0.75 M to about 1.5 M NaCl, about 1.0 M NaCl to about 1.5 M NaCl, about 1.25 M NaCl to about 1.5 M NaCl, about 0.5 M NaCl to about 1.25 M NaCl, about 0.5 M NaCl to about 1.0 M NaOH, or about 0.5 M NaCl to about 0.75 M NaCl. In some embodiments, sanitization buffer comprises about any of 0.5 M, 0.75 M, 1.0 M, 1.25 M, or 1.5 M NaCl. In some embodiments, sanitization buffer comprises about 0.5 N NaOH and about 1 M NaCl.

In some embodiments, about 3 to about 12, about 3 to about 11, about 3 to about 10, about 3 to about 9, about 3 to about 8, about 3 to about 7, about 3 to about 6, about 3 to about 5, about 3 to about 4, about 4 to about 12, about 5 to about 12, about 6 to about 12, about 7 to about 12, about 8 to about 12, about 9 to about 12, about 10 to about 12, about 11 to about 12, about 5 to about 12, about 5 to about 10, about 8 to about 12, or about 9 to about 11 material volumes (e.g., column volumes) of sanitization buffer are passed through the ion exchange chromatography material. In some embodiments, about any of 3, 4, 5, 6, 7, 8, 9, 10, 11, or 12 material volumes (e.g., column volumes) of sanitization buffer are passed through the ion exchange chromatography material. In some embodiments, about 10 material volumes (e.g., column volumes) of sanitization buffer are passed through the ion exchange chromatography material. In some embodiments, about 4 material volumes (e.g., column volumes) of sanitization buffer are passed through the ion exchange chromatography material.

In some embodiments, the storage buffer comprises NaOH. In some embodiments, the storage buffer comprises about 0.05 N to about 0.5 N NaOH, 0.05 N to about 0.4 N NaOH, 0.05 N to about 0.4 N NaOH, 0.05 N to about 0.3 N NaOH, 0.05 N to about 0.2 N NaOH, 0.05 N to about 0.1 N NaOH, 0.1 N to about 0.5 N NaOH, 0.2 N to about 0.5 N NaOH, 0.3 N to about 0.05 N NaOH, or 0.4 N to about 0.5 N NaOH. In some embodiments, storage buffer comprises about any of 0.05 N, 0.06 N, 0.07 N NaOH, 0.08 N, 0.09 N, 0.1 N, 0.2 N, 0.3 N, 0.4 N or 0.5 N NaOH. In some embodiments, storage buffer comprises about 0.1 N NaOH.

In some embodiments, the ion exchange chromatography material is stored in about 1 to about 10, about 1 to about 8, about 1 to about 6, about 1 to about 5, about 1 to about 4, about 1 to about 3, about 1 to about 2, about 2 to about 10, about 3 to about 20, about 4 to about 10, about 5 to about 10, about 1 to about 7, about 2 to about 6, about 3 to about 5 material volumes (e.g., column volumes) of storage buffer. In some embodiments, the ion exchange chromatography material is stored in about any of 1, 2, 3, 4, 5, 6, 7, 8, 9, or 10 material volumes (e.g., column volumes) of storage buffer. In some embodiments, the ion exchange chromatography material is stored in about 4 material volumes (e.g., column volumes) of storage buffer. In some embodiments, the ion exchange chromatography material is stored in about 3 material volumes (e.g., column volumes) of storage buffer.

In some aspects, the invention provides methods to clean an ion exchange chromatography material for reuse wherein the chromatography material is used in overload mode in the purification of a polypeptide, the method comprising the steps of a) passing a regeneration buffer through the ion exchange chromatography material, wherein the regeneration buffer comprises about 350 mM sodium acetate at about pH 8.3; b) passing of sanitization buffer through the ion exchange chromatography material, wherein the sanitization buffer comprises about 0.5 N NaOH, and c) storing the ion exchange chromatography material in a storage buffer, wherein the storage buffer comprises about 0.1 N NaOH. In some aspects, the invention provides methods to clean an ion exchange chromatography material for reuse wherein the chromatography material is used in overload mode in the purification of a polypeptide, the method comprising the steps of a) passing about 10 material volumes of a regeneration buffer through the ion exchange chromatography material, wherein the regeneration buffer comprises about 350 mM sodium acetate at about pH 8.3; b) passing about 10 material volumes of sanitization buffer through the ion exchange chromatography material, wherein the sanitization buffer comprises about 0.5 N NaOH; and c) storing the ion exchange chromatography material in about 4 material volumes of a storage buffer, wherein the storage buffer comprises about 0.1 N NaOH. In some embodiments, the ion exchange chromatography material is a cation exchange chromatography material. In some embodiments, the ion exchange chromatography material is an anion exchange chromatography material.

In some aspects, the invention provides methods to clean a cation exchange chromatography material for reuse wherein the chromatography material is used in overload mode in the purification of a polypeptide, the method comprising the steps of a) passing a regeneration buffer through the cation exchange chromatography material, wherein the regeneration buffer comprises about 350 mM sodium acetate at about pH 8.3; b) passing of sanitization buffer through the cation exchange chromatography material, wherein the sanitization buffer comprises about 0.5 N NaOH; and c) storing the cation exchange chromatography material in a storage buffer, wherein the storage buffer comprises about 0.1 N NaOH. In some aspects, the invention provides methods to clean a cation exchange chromatography material for reuse wherein the chromatography material is used in overload mode in the purification of a polypeptide, the method comprising the steps of a) passing about 10 material volumes of a regeneration buffer through the cation exchange chromatography material, wherein the regeneration buffer comprises about 350 mM sodium acetate at about pH 8.3; b) passing about 10 material volumes of sanitization buffer through the cation exchange chromatography material, wherein the sanitization buffer comprises about 0.5 N NaOH; and c) storing the cation exchange chromatography material in about 4 material volumes of a storage buffer, wherein the storage buffer comprises about 0.1 N NaOH.

In some aspects, the invention provides methods to clean an anion exchange chromatography material for reuse wherein the chromatography material is used in overload mode in the purification of a polypeptide, the method comprising the steps of a) passing a regeneration buffer through the anion exchange chromatography material, wherein the regeneration buffer comprises about 350 mM sodium acetate at about pH 8.3; b) passing of sanitization buffer through the anion exchange chromatography material, wherein the sanitization buffer comprises about 0.5 N NaOH; and c) storing the anion exchange chromatography material in a storage buffer, wherein the storage buffer comprises about 0.1 N NaOH. In some aspects, the invention provides methods to clean an anion exchange chromatography material for reuse wherein the chromatography material is used in overload mode in the purification of a polypeptide, the method comprising the steps of a) passing about 10 material volumes of a regeneration buffer through the anion exchange chromatography material, wherein the regeneration buffer comprises about 350 mM sodium acetate at about pH 8.3, b) passing about 10 material volumes of sanitization buffer through the anion exchange chromatography material, wherein the sanitization buffer comprises about 0.5 N NaOH; and c) storing the anion exchange chromatography material in about 4 material volumes of a storage buffer, wherein the storage buffer comprises about 0.1 N NaOH.

In some embodiments, the invention provides a method to clean an ion exchange chromatography material for reuse wherein the chromatography material is used in overload mode in the purification of a polypeptide, the method comprising the steps of a) passing a regeneration buffer through the ion exchange chromatography material, wherein the regeneration buffer comprises about 25 mM Tris and about 85 mM sodium acetate at about pH 8.0; b) passing a sanitization buffer through the ion exchange chromatography material, wherein the sanitization buffer comprises about 0.5 N NaOH; and c) storing the ion exchange chromatography material in a storage buffer, wherein the storage buffer comprises about 0.1 N NaOH. In some embodiments, the invention provides a method to clean an ion exchange chromatography material for reuse wherein the chromatography material is used in overload mode in the purification of a polypeptide, the method comprising the steps of a) passing about 10 material volumes of a regeneration buffer through the ion exchange chromatography material, wherein the regeneration buffer comprises about 25 mM Tris and about 85 mM sodium acetate at about pH 8.0; b) passing about 10 material volumes of a sanitization buffer through the ion exchange chromatography material, wherein the sanitization buffer comprises about 0.5 N NaOH; and c) storing the ion exchange chromatography material in about 4 material volumes of a storage buffer, wherein the storage buffer comprises about 0.1 N NaOH. In some embodiments, the ion exchange chromatography material is a cation exchange chromatography material. In some embodiments, the ion exchange chromatography material is an anion exchange chromatography material.

In some embodiments, the invention provides a method to clean a cation exchange chromatography material for reuse wherein the chromatography material is used in overload mode in the purification of a polypeptide, the method comprising the steps of a) passing a regeneration buffer through the cation exchange chromatography material, wherein the regeneration buffer comprises about 25 mM Tris and about 85 mM sodium acetate at about pH 8.0; b) passing a sanitization buffer through the cation exchange chromatography material, wherein the sanitization buffer comprises about 0.5 N NaOH; and c) storing the cation exchange chromatography material in a storage buffer, wherein the storage buffer comprises about 0.1 N NaOH. In some embodiments, the invention provides a method to clean a cation exchange chromatography material for reuse wherein the chromatography material is used in overload mode in the purification of a polypeptide, the method comprising the steps of a) passing about 10 material volumes of a regeneration buffer through the cation exchange chromatography material, wherein the regeneration buffer comprises about 25 mM Tris and about 85 mM sodium acetate at about pH 8.0; b) passing about 10 material volumes of a sanitization buffer through the cation exchange chromatography material, wherein the sanitization buffer comprises about 0.5 N NaOH; and c) storing the cation exchange chromatography material in about 4 material volumes of a storage buffer, wherein the storage buffer comprises about 0.1 N NaOH.

In some embodiments, the invention provides a method to clean an anion exchange chromatography material for reuse wherein the chromatography material is used in overload mode in the purification of a polypeptide, the method comprising the steps of a) passing a regeneration buffer through the anion exchange chromatography material, wherein the regeneration buffer comprises about 25 mM Tris and about 85 mM sodium acetate at about pH 8.0; b) passing a sanitization buffer through the anion exchange chromatography material, wherein the sanitization buffer comprises about 0.5 N NaOH; and c) storing the anion exchange chromatography material in a storage buffer, wherein the storage buffer comprises about 0.1 N NaOH. In some embodiments, the invention provides a method to clean an anion exchange chromatography material for reuse wherein the chromatography material is used in overload mode in the purification of a polypeptide, the method comprising the steps of a) passing about 10 material volumes of a regeneration buffer through the anion exchange chromatography material, wherein the regeneration buffer comprises about 25 mM Tris and about 85 mM sodium acetate at about pH 8.0; b) passing about 10 material volumes of a sanitization buffer through the anion exchange chromatography material, wherein the sanitization buffer comprises about 0.5 N NaOH; and c) storing the anion exchange chromatography material in about 4 material volumes of a storage buffer, wherein the storage buffer comprises about 0.1 N NaOH.

In some aspects, the invention provides methods for purifying a polypeptide from a composition comprising the polypeptide and an impurity using an ion exchange chromatography material wherein the ion exchange chromatography material is suitable for reuse, the method comprising the steps of a) equilibrating an ion exchange chromatography material with an equilibration buffer; b) loading the composition on the ion exchange chromatography material, wherein the load density is ≤about 1000 g/L; c) collecting fractions comprising the polypeptide; d) washing the ion exchange chromatography material with equilibration buffer; e) passing a regeneration buffer through the ion exchange chromatography material; f) passing of sanitization buffer through the ion exchange chromatography material and g) storing the ion exchange chromatography material in a storage buffer. In some embodiments, the ion exchange chromatography material is a cation exchange chromatography material. In some embodiments, the ion exchange chromatography material is an anion exchange chromatography material.

In some aspects, the invention provides methods for purifying a polypeptide from a composition comprising the polypeptide and an impurity using a cation exchange chromatography material wherein the cation exchange chromatography material is suitable for reuse, the method comprising the steps of a) equilibrating a cation exchange chromatography material with an equilibration buffer; b) loading the composition on the cation exchange chromatography material, wherein the load density is ≤about 1000 g/L; c) collecting fractions comprising the polypeptide; d) washing the cation exchange chromatography material with equilibration buffer; e) passing a regeneration buffer through the cation exchange chromatography material; f) passing of sanitization buffer through the cation exchange chromatography material and g) storing the cation exchange chromatography material in a storage buffer.

In some aspects, the invention provides methods for purifying a polypeptide from a composition comprising the polypeptide and an impurity using an anion exchange chromatography material wherein the anion exchange chromatography material is suitable for reuse, the method comprising the steps of a) equilibrating an anion exchange chromatography material with an equilibration buffer; b) loading the composition on the anion exchange chromatography material, wherein the load density is ≤about 1000 g/L; c) collecting fractions comprising the polypeptide; d) washing the anion exchange chromatography material with equilibration buffer; e) passing a regeneration buffer through the anion exchange chromatography material; f) passing of sanitization buffer through the anion exchange chromatography material and g) storing the anion exchange chromatography material in a storage buffer.

In some aspects, the invention provides methods for purifying a polypeptide from a composition comprising the polypeptide and an impurity using an ion exchange chromatography material wherein the ion exchange chromatography material is suitable for reuse, the method comprising the steps of a) equilibrating an ion exchange chromatography material with an equilibration buffer; b) loading the composition on the ion exchange chromatography material, wherein the load density is ≥20 g/L; c) collecting fractions comprising the polypeptide; d) washing the ion exchange chromatography material with equilibration buffer; e) passing a regeneration buffer through the ion exchange chromatography material; f) passing of sanitization buffer through the ion exchange chromatography material and g) storing the ion exchange chromatography material in a storage buffer. In some embodiments, the ion exchange chromatography material is a cation exchange chromatography material. In some embodiments, the ion exchange chromatography material is an anion exchange chromatography material.

In some aspects, the invention provides methods for purifying a polypeptide from a composition comprising the polypeptide and an impurity using a cation exchange chromatography material wherein the cation exchange chromatography material is suitable for reuse, the method comprising the steps of a) equilibrating a cation exchange chromatography material with an equilibration buffer; b) loading the composition on the cation exchange chromatography material, wherein the load density is ≥20 g/L; c) collecting fractions comprising the polypeptide; d) washing the cation exchange chromatography material with equilibration buffer; e) passing a regeneration buffer through the cation exchange chromatography material; f) passing of sanitization buffer through the cation exchange chromatography material and g) storing the cation exchange chromatography material in a storage buffer.

In some aspects, the invention provides methods for purifying a polypeptide from a composition comprising the polypeptide and an impurity using an anion exchange chromatography material wherein the anion exchange chromatography material is suitable for reuse, the method comprising the steps of a) equilibrating an anion exchange chromatography material with an equilibration buffer; b) loading the composition on the anion exchange chromatography material, wherein the load density is ≥20 g/L; c) collecting fractions comprising the polypeptide; d) washing the anion exchange chromatography material with equilibration buffer; e) passing a regeneration buffer through the anion exchange chromatography material; f) passing of sanitization buffer through the anion exchange chromatography material and g) storing the anion exchange chromatography material in a storage buffer.

In some aspects, the invention provides methods for purifying a polypeptide from a composition comprising the polypeptide and an impurity using an ion exchange chromatography material wherein the ion exchange chromatography material is suitable for reuse, the method comprising the steps of a) equilibrating an ion exchange chromatography material with an equilibration buffer; b) loading the composition on the ion exchange chromatography material, wherein the load density exceeds the dynamic binding capacity of the ion exchange chromatography material for the polypeptide; c) collecting fractions comprising the polypeptide; d) washing the ion exchange chromatography material with equilibration buffer; e) passing a regeneration buffer through the ion exchange chromatography material; f) passing of sanitization buffer through the ion exchange chromatography material and g) storing the ion exchange chromatography material in a storage buffer. In some embodiments, the ion exchange chromatography material is a cation exchange chromatography material. In some embodiments, the ion exchange chromatography material is an anion exchange chromatography material.

In some aspects, the invention provides methods for purifying a polypeptide from a composition comprising the polypeptide and an impurity using a cation exchange chromatography material wherein the cation exchange chromatography material is suitable for reuse, the method comprising the steps of a) equilibrating a cation exchange chromatography material with an equilibration buffer; b) loading the composition on the cation exchange chromatography material, wherein the load density exceeds the dynamic binding capacity of the cation exchange chromatography material for the polypeptide; c) collecting fractions comprising the polypeptide; d) washing the cation exchange chromatography material with equilibration buffer; e) passing a regeneration buffer through the cation exchange chromatography material; f) passing of sanitization buffer through the cation exchange chromatography material and g) storing the cation exchange chromatography material in a storage buffer.

In some aspects, the invention provides methods for purifying a polypeptide from a composition comprising the polypeptide and an impurity using an anion exchange chromatography material wherein the anion exchange chromatography material is suitable for reuse, the method comprising the steps of a) equilibrating an anion exchange chromatography material with an equilibration buffer; b) loading the composition on the anion exchange chromatography material, wherein the load density exceeds the dynamic binding capacity of the anion exchange chromatography material for the polypeptide; c) collecting fractions comprising the polypeptide; d) washing the anion exchange chromatography material with equilibration buffer; e) passing a regeneration buffer through the anion exchange chromatography material; f) passing of sanitization buffer through the anion exchange chromatography material and g) storing the anion exchange chromatography material in a storage buffer.

In some embodiments, the equilibration buffer comprises about 10 mM to about 100 mM, about 20 mM to about 100 mM, about 30 mM to about 100 mM, about 40 mM to about 100 mM, about 10 mM to about 90 mM, about 10 mM to about 80 mM, about 10 mM to about 70 mM, about 10 mM to about 60 mM, about 10 mM to about 50 mM, about 10 mM to about 40 mM, about 10 mM to about 70 mM, about 20 mM to about 60 mM, or about 30 mM to about 50 mM NaOAc. In some embodiments, the equilibration buffer comprises about any of 10 mM, 20 mM, 30 mM, 40 mM, 50 mM, 60 mM, 70 mM, 80 mM, 90 mM, or 100 mM NaOAc. In some embodiments, the equilibration buffer comprises about 40 mM NaOAc.

In some embodiments, the pH of the equilibration buffer is about 4.0 to about 8.0, about 5.0 to about 8.0, about 6.0 to about 8.0, about 7.0 to about 8.0, about 4.0 to about 7.0, about 4.0 to about 6.0, about 4.0 to about 5.0, about 4.0 to about 7.0, about 4.5 to about 6.5, or about 5.0 to about 6.0. In some embodiments, the pH of the equilibration buffer is about any of 4.0, 4.5, 5.0, 5.5, 6.0, 6.5, 7.0, 7.5 or 8.0. In some embodiments, the pH of the equilibration buffer is about 5.5.

In some embodiments, about 3 to about 10, about 3 to about 9, about 3 to about 8, about 3 to about 7, about 3 to about 6, about 3 to about 5, about 3 to about 4, about 4 to about 10, about 5 to about 10, about 6 to about 10, about 7 to about 10, about 8 to about 10, about 9 to about 10, about 3 to about 8, about 4 to about 7, or about 5 to about 6 material volumes (e.g., column volumes) of equilibration buffer are passed through the ion exchange chromatography material prior to loading the composition. In some embodiments, about any of 3, 4, 5, 6, 7, 8, 9, or 10 material volumes (e.g., column volumes) of equilibration buffer are passed through the ion exchange chromatography material prior to loading the composition. In some embodiments, about 5 material volumes (e.g., column volumes) of equilibration buffer are passed through the ion exchange chromatography material prior to loading the composition. In some embodiments, the ion exchange material is washed with equilibration buffer after loading the composition until a sufficient amount of polypeptide is collected in the fractions.

In some embodiments, the regeneration buffer comprises about 50 to about 600 mM NaOAc. In some embodiments, the regeneration buffer comprises about 350 mM NaOAc. In some embodiments, the regeneration buffer comprises about 25 to about 75 mM Tris and about 50 to about 100 mM NaOAc. In some embodiments, the regeneration buffer comprises about 50 mM Tris and about 85 mM NaOAc. In some embodiments, the pH of the regeneration buffer is about pH 4.0 to 11.0. In some embodiments, the pH of the regeneration buffer is about pH 8.3. In some embodiments, the pH of the regeneration buffer is about pH 8.0. In some embodiments, the regeneration buffer comprises about 350 mM NaOAc at about pH 8.3. In some embodiments, the regeneration buffer comprises about 50 mM Tris and about 85 mM NaOAc at about pH 8.0. In some embodiments, about 3 to about 12 material volumes of regeneration buffer are passed through the ion exchange chromatography material. In some embodiments, about 10 material volumes of regeneration buffer are passed through the ion exchange chromatography material.

In some embodiments, the sanitization buffer comprises about 0.1 N to about 1 N NaOH. In some embodiments, the sanitization buffer comprises about 0.5 N NaOH. In some embodiments, about 2 to about 12 material volumes of sanitization buffer are passed through the ion exchange chromatography material. In some embodiments, about 10 material volumes of sanitization buffer am passed passing a regeneration buffer through the ion exchange chromatography material.

In some embodiments, the storage buffer comprises about 0.05 N to about 0.5 N NaOH. In some embodiments, the sanitization buffer comprises about 0.1 N NaOH. In some embodiments the ion exchange chromatography material is stored in about 1 to 5 material volumes of storage buffer. In some embodiments, the ion exchange chromatography material is stored in about 4 material volumes of storage buffer.

In some embodiments, the load density of the composition is ≤1000 g/L chromatography material. In some embodiments, the load density of the composition is any of about ≤1000 g/L, ≤900 g/L, ≤800 g/L, ≤750 g/L, ≤700 g/L, ≤600 g/L, ≤500 g/L, ≤400 g/L, ≤300 g/L, ≤250 g/L, ≤200 g/L, ≤100 g/L chromatography material. In some embodiments, the load density of the composition is between any of about 20 g/L and about 1000 g/L, about 50 g/L and about 1000 g/L, about 100 g/L and about 1000 g/L, about 200 g/L and about 1000 g/L, about 250 g/L and about 1000 g/L, about 300 g/L and about 1000 g/L, about 400 g/L and about 1000 g/L, about 500 g/L and about 1000 g/L, about 600 g/L and about 1000 g/L, about 700 g/L and about 1000 g/L, about 750 g/L and about 1000 g/L, about 800 g/L and about 1000 g/L, or about 900 g/L and about 1000 g/L, chromatography material. In some embodiments, the load density of the composition is between any of about 20 g/L and about 900 g/L, about 20 g/L and about 800 g/L, about 20 g/L and about 750 g/L, about 20 g/L and about 700 g/L, about 20 g/L and about 600 g/L, about 20 g/L and about 500 g/L, about 20 g/L and about 400 g/L, about 20 g/L and about 300 g/L, about 20 g/L and about 250 g/L, about 20 g/L and about 200 g/L, about 20 g/L and about 100 g/L, about 20 g/L and about 75 g/L, or about 20 g/L and about 50 g/L, chromatography material.

In some embodiments, the load density of the composition is ≥20 g/L chromatography material. In some embodiments, the load density of the composition is any of about ≥20 g/L, ≥50 g/L, ≥75 g/L, ≥100 g/L, ≥200 g/L, ≥250 g/L, ≥300 g/L, ≥400 g/L, ≥500 g/L, ≥600 g/L, ≥700 g/L, ≥750 g/L, ≥800 g/L, ≥900 g/L, ≥1000 g/L, ≥1200 g/L, ≥1400 g/L, ≥1500 g/L, ≥1750 g/L, or ≥2000 g/L chromatography material. In some embodiments, the load density of the composition is between any of about 20 g/L and about 2000 g/L, about 50 g/L and about 2000 g/L, about 100 g/L and about 2000 g/L, about 200 g/L and about 2000 g/L, about 250 g/L and about 2000 g/L, about 300 g/L and about 2000 g/L, about 400 g/L and about 2000 g/L, about 500 g/L and about 2000 g/L, about 600 g/L and about 2000 g/L, about 700 g/L and about 2000 g/L, about 750 g/L and about 2000 g/L, about 800 g/L and about 2000 g/L, about 900 g/L and about 2000 g/L, about 1000 g/L and about 2000 g/L, about 1250 g/L and about 2000 g/L. or about 1500 g/L and about 2000 g/L chromatography material. In some embodiments, the load density of the composition is between any of about 20 g/L and about 1750 g/L, about 20 g/L and about 1500 g/L, about 20 g/L and about 1250 g/L, about 20 g/L and about 1200 g/L, or about 20 g/L and about 1100 g/L chromatography material.

In some embodiments, the load density of the composition exceeds the dynamic binding capacity of the chromatography material for the polypeptide. In some embodiments, the load density of the composition exceeds the dynamic binding capacity of the chromatography material for the polypeptide by about any of 2× (2-fold), 3×, 4×, 5×, 6×, 7×, 8×, 9×, 10×, 11×, 12×, 13×, 14×, 15×, 20×, 25×, 30×, 40×, 50×, 60×, 70×, 75×, 80×, 90×, or 100×. In some embodiments, the load density of the composition exceeds the dynamic binding capacity of the chromatography material for the polypeptide by between 2× and 100×, 5× and 100×, 10× and 100×, 20× and 100×, 25× and 100×, 30× and 100×, 40× and 100×, 50× and 100×, 60× and 100×, 70× and 100×, 75× and 100×, 80× and 100×, or 90× and 100×. In some embodiments, the load density of the composition exceeds the dynamic binding capacity of the chromatography material for the polypeptide by between 2× and 100×, 2× and 75×, 2× and 50×, 2× and 25×, 2× and 20×, 2× and 15×, 2× and 14×, 2× and 13×, 2× and 12×, 2× and 11×, 2× and 10×, 2× and 9×, 2× and 8×, 2× and 7×, 2× and 6×, 2× and 5×, 2× and 4×, or 2× and 3×.

In some aspects, the invention provides a method for purifying a polypeptide from a composition comprising the polypeptide and an impurity using an ion exchange chromatography material wherein the ion exchange chromatography material is suitable for reuse, the method comprising the steps of a) equilibrating an ion exchange chromatography material with an equilibration buffer; b) loading the composition on the ion exchange chromatography material, wherein the load density exceeds the dynamic binding capacity of the ion exchange chromatography for the polypeptide; c) collecting fractions comprising the polypeptide; d) washing the ion exchange chromatography material with equilibration buffer; e) passing a regeneration buffer through the ion exchange chromatography material, wherein the regeneration buffer comprises sodium acetate; f) passing an acid wash through the ion exchange chromatography material, g) passing a sanitization buffer through the ion exchange chromatography material, wherein the sanitization buffer comprises about 0.5 N NaOH and about 1 M NaCl; and h) storing the ion exchange chromatography material in a storage buffer, wherein the storage buffer comprises about 0.1 N NaOH. In some embodiments, the ion exchange chromatography material is a cation exchange chromatography material. In some embodiments, the ion exchange chromatography material is an anion exchange chromatography material. In some embodiments, the ion exchange chromatography material is a mixed mode anion exchange chromatography material. In some embodiments, the ion exchange chromatography material is a Capto™ Adhere chromatography material.

In some embodiments, the equilibration buffer comprises about 50 mM to about 100 mM, about 55 mM to about 100 mM, about 60 mM to about 100 mM, about 65 mM to about 100 mM, about 70 mM to about 100 mM, about 75 mM to about 100 mM, about 80 mM to about 100 mM, about 85 mM to about 100 mM, about 90 mM to about 100 mM, about 95 mM to about 100 mM, about 50 mM to about 95 mM, about 50 mM to about 90 mM, about 50 mM to about 85 mM, about 50 mM to about 80 mM, about 50 mM to about 75 mM, about 50 mM to about 70 mM, about 50 mM to about 65 mM, about 50 mM to about 60 mM, or about 50 mM to about 55 mM NaOAc. In some embodiments, the equilibration buffer comprises about any of 50 mM, 55 mM, 60 mM, 65 mM, 70 mM, 75 mM, 80 mM, 85 mM, 90 mM, 95 mM, or 100 mM NaOAc. In some embodiments, the equilibration buffer comprises about 85 mM NaOAc. In some embodiments, the equilibration buffer comprises about 20 mM to about 80 mM, about 30 mM to about 80 mM, about 40 mM to about 80 mM, about 50 mM to about 80 mM, about 60 mM to about 80 mM, about 70 mM to about 80 mM, about 20 mM to about 70 mM, about 20 mM to about 60 mM, about 20 mM to about 50 mM, about 20 mM to about 40 mM, or about 20 mM to about 30 mM Tris. In some embodiments, the equilibration buffer comprises about any of 25 mM, 30 mM, 35 mM, 40 mM, 45 mM, 50 mM, 55 mM, 60 mM, 65 mM, 70 mM, or 70 mM Tris. In some embodiments, the equilibration buffer comprises about 50 mM Tris. In some embodiments, the equilibration buffer comprises about 50 mM Tris and 85 mM NaOAc.

In some embodiments, the pH of the equilibration buffer is about 6.0 to about 10.0, about 7.0 to about 10.0, about 8.0 to about 10.0, about 9.0 to about 10.0, about 9.0 to about 9.0, about 6.0 to about 8.0, or about 6.0 to about 7.0. In some embodiments, the pH of the equilibration buffer is about any of 6.0, 6.5, 7.0, 7.5, 8.0, 8.5, 9.0, 9.5 or 10.0. In some embodiments, the pH of the equilibration buffer is about 8.0. In some embodiments, the equilibration buffer comprises about 50 mM Tris and 85 mM NaOAc, about pH 8.0.

In some embodiments, about 3 to about 10, about 3 to about 9, about 3 to about 8, about 3 to about 7, about 3 to about 6, about 3 to about 5, about 3 to about 4, about 4 to about 10, about 5 to about 10, about 6 to about 10, about 7 to about 10, about 8 to about 10, about 9 to about 10, about 3 to about 8, about 4 to about 7, or about 5 to about 6 material volumes (e.g., column volumes) of equilibration buffer are passed through the ion exchange chromatography material prior to loading the composition. In some embodiments, about any of 3, 4, 5, 6, 7, 8, 9, or 10 material volumes (e.g., column volumes) of equilibration buffer are passed through the ion exchange chromatography material prior to loading the composition. In some embodiments, about 5 material volumes (e.g., column volumes) of equilibration buffer are passed through the ion exchange chromatography material prior to loading the composition. In some embodiments, the ion exchange material is washed with equilibration buffer after loading the composition until a sufficient amount of polypeptide is collected in the fractions. In some embodiments, about any of 5, 6, 7, 8, 9, 10, 11, 12, 13, 14, or 15 material volumes (e.g., column volumes) of equilibration buffer are passed through the ion exchange chromatography material after loading the composition. In some embodiments, about 10 material volumes (e.g., column volumes) of equilibration buffer are passed through the ion exchange chromatography material after loading the composition.

In some embodiments, the regeneration buffer comprises about 50 to about 600 mM NaOAc. In some embodiments, the regeneration buffer comprises NaOAc at a concentration range of about any of 25 mM to 600 mM, 30 mM to 600 mM, 40 mM to 600 mM, 50 mM to 600 mM, 75 mM to 600 mM, 100 mM to 600 mM, 125 mM to 600 mM, 150 mM to 600 mM, 175 mM to 600 mM, 200 mM to 600 mM, 225 mM to 600 mM, 250 mM to 600 mM, 275 mM to 600 mM, 300 mM to 600 mM, 325 mM to 600 mM, 350 mM to 600 mM, 375 mM to 600 mM, 400 mM to 600 mM, 450 mM to 600 mM, 500 mM to 600 mM, 550 mM to 600 mM, 25 mM to 550 mM, 25 mM to 500 mM, 25 mM to 475 mM, 25 mM to 450 mM, 25 mM to 425 mM, 25 mM to 400 mM, 25 mM to 375 mM, 25 mM to 550 mM, 25 mM to 325 mM, 25 mM to 300 mM, 25 mM to 275 mM, 25 mM to 250 mM, 25 mM to 225 mM, 25 mM to 200 mM, 25 mM to 175 mM, 25 mM to 150 mM, 25 mM to 125 mM, 25 mM to 100 mM, 25 mM to 90 mM, 25 mM to 80 mM, 25 mM to 70 mM, 25 mM to 60 mM, 25 mM to 50 mM, 25 mM to 40 mM, or 25 mM to 30 mM. In some embodiments, the regeneration buffer comprises about any of 10, 20, 30, 40, 50, 60, 70, 80, 90, 100, 150, 200, 250, 300, 350, 400, 450, 500, 550, or 600 mM NaOAc. In some embodiments, the regeneration buffer comprises about 350 mM NaOAc. In some embodiments, the regeneration buffer comprises about 40 mM NaOAc.

In some embodiments, the pH of the regeneration buffer is about pH 2.0 to about pH 7.0. In some embodiments, the pH of the regeneration buffer is about pH 2.0 to about pH 6.5, about pH 2.0 to about pH 6.0, about pH 2.0 to about pH 5.5, about pH 2.0 to about pH 5.0, about pH 2.0 to about pH 4.5, about pH 2.0 to about pH 4.0, about pH 2.0 to about pH 3.5, about pH 2.0 to about pH 3.0, about pH 2.0 to about pH 2.5, about pH 2.5 to about pH 7.0, about pH 3.0 to about pH 7.0, about pH 3.5 to about pH 7.0, about pH 4.0 to about pH 7.0, about pH 4.5 to about pH 7.0, about pH 5.0 to about pH 7.0, about pH 5.5 to about pH 7.0, about pH 6.0 to about pH 7.0, or about pH 6.5 to about pH 7.0. In some embodiments, the regeneration buffer is about pH 2.0, about pH 8.0, about pH 2.5, about pH 3.0, about pH 3.5, about pH 4.0, about pH 4.5, about pH 5.0, about pH 5.5, about pH 6.0, about pH 6.5, or about pH 7.0. In some embodiments, the pH of regeneration buffer is about 4.0. In some embodiments, the regeneration buffer is about 350 mM NaOAc at about pH 4.0. In some embodiments, the regeneration buffer is about 350 mM NaOAc at about pH 5.0. In some embodiments, the regeneration buffer is about 40 mM NaOAc at about pH 4.0. In some embodiments, the regeneration buffer is about 40 mM NaOAc at about pH 4.0.

In some embodiments, about 3 to about 12, about 3 to about 11, about 3 to about 10, about 3 to about 9, about 3 to about 8, about 3 to about 7, about 3 to about 6, about 3 to about 5, about 3 to about 4, about 4 to about 12, about 5 to about 12, about 6 to about 12, about 7 to about 12, about 8 to about 12, about 9 to about 12, about 10 to about 12, about 11 to about 12, about 5 to about 12, about 5 to about 10, about 8 to about 12, or about 9 to about 11 material volumes (e.g., column volumes) of the regeneration buffer are passed through the ion exchange chromatography material. In some embodiments, about any of 3, 4, 5, 6, 7, 8, 9, 10, 11, or 12 material volumes (e.g., column volumes) of the regeneration buffer are passed through the ion exchange chromatography material. In some embodiments, about 7 material volumes (e.g., column volumes) of regeneration buffer are passed through the ion exchange chromatography material.

In some embodiments, the acid wash comprises NaOAc. In some embodiments, the acid wash comprises phosphoric acid. In some embodiments, the acid wash comprises NaOAc and phosphoric acid. In some embodiments, the concentration of NaOAc in the acid wash is more than about any of 0.1M to less than about 0.5 M. In some embodiments, the concentration of NaOAc in the acid wash is any of about 0.100 M, 0.120M, 0.130M, 0.140M, 0.150M, 0.160M, 0.161M, 0.162M, 0.163M, 0.164M, 0.167M, 0.168M, 0.169M, 0.170M, 0.180M, 0.190M, 0.200M, 0.250M, 0.300M, 0.350M, 0.400M, 0.450M, or 0.500M. In some embodiments, the concentration of phosphoric acid in the acid wash is any of about 0.10 M, 0.15M, 0.20M, 0.25M, 0.30M, 0.35M, 0.40M, 0.45M, 0.50M, 0.55M, or 0.60M. In some embodiments, the acid wash comprises about 0.167M NaOAc and about 0.3M phosphoric acid.

In some embodiments, about 1 to about 5, about 1 to about 4, about 1 to about 3, about 1 to about 2, about 2 to about 5, about 2 to about 4, about 2 to about 3, about 3 to about 5, about 3 to about 4, about 4 to about 5 material volumes (e.g., column volumes) of acid wash are passed through the ion exchange chromatography material. In some embodiments, about any of 1, 2, 3, 4, or 5 material volumes (e.g., column volumes) of acid wash are passed through the ion exchange chromatography material. In some embodiments, about 3 material volumes (e.g., column volumes) of acid wash are passed through the ion exchange chromatography material.

In some embodiments, the sanitization buffer comprises about 0.1 N to about 1 N NaOH In some embodiments, the sanitization buffer comprises about 0.5 N NaOH. In some embodiments, the sanitization buffer comprises about 0.5 M to about 1.5 M NaCl. In some embodiments, the sanitization buffer comprises about 0.5 M NaCl to about 2 M NaCl. In some embodiments, the sanitization buffer comprises about 1.0 M NaCl. In some embodiments, the sanitization buffer comprises about 0.5 N NaOH and about M NaCl. In some embodiments, about 1 to about 10 material volumes of sanitization buffer are passed through the ion exchange chromatography material. In some embodiments, about 4 material volumes of sanitization buffer are passed through the ion exchange chromatography material.

In some embodiments, the storage buffer comprises about 0.05 N to about 0.5 N NaOH. In some embodiments, the sanitization buffer comprises about 0.1 N NaOH. In some embodiments, the ion exchange chromatography material is stored in about 1 to 5 material volumes of storage buffer. In some embodiments, the ion exchange chromatography material is stored in about 3 material volumes of storage buffer.

In some embodiments, the load density of the composition is ≤1000 g/L chromatography material. In some embodiments, the load density of the composition is any of about ≤1000 g/L, ≤900 g/L, ≤800 g/L, ≤750 g/L, ≤700 g/L, ≤600 g/L, ≤500 g/L, ≤400 g/L, ≤300 g/L, ≤250 g/L, ≤200 g/L, ≤100 g/L chromatography material. In some embodiments, the load density of the composition is between any of about 20 g/L and about 1000 g/L, about 50 g/L and about 1000 g/L, about 100 g/L and about 1000 g/L, about 200 g/L and about 1000 g/L, about 250 g/L and about 1000 g/L, about 300 g/L and about 1000 g/L, about 400 g/L and about 1000 g/L, about 500 g/L and about 1000 g/L, about 600 g/L and about 1000 g/L, about 700 g/L and about 1000 g/L, about 750 g/L and about 1000 g/L, about 800 g/L and about 1000 g/L, or about 900 g/L and about 1000 g/L, chromatography material. In some embodiments, the load density of the composition is between any of about 20 g/L and about 900 g/L, about 20 g/L and about 800 g/L, about 20 g/L and about 750 g/L, about 20 g/L and about 700 g/L, about 20 g/L and about 600 g/L, about 20 g/L and about 500 g/L, about 20 g/L and about 400 g/L, about 20 g/L and about 300 g/L, about 20 g/L and about 250 g/L, about 20 g/L and about 200 g/L, about 20 g/L and about 100 g/L, about 20 g/L and about 75 g/L, or about 20 g/L and about 50 g/L, chromatography material.

In some embodiments, the load density of the composition is ≥20 g/L chromatography material. In some embodiments, the load density of the composition is any of about ≥20 g/L, ≥50 g/L, ≥75 g/L, ≥100 g/L, ≥200 g/L, ≥250 g/L, ≥300 g/L, ≥400 g/L, ≥500 g/L, ≥600 g/L, ≥700 g/L, ≥750 g/L, ≥800 g/L, ≥900 g/L, ≥1000 g/L, ≥1200 g/L, ≥1400 g/L, ≥1500 g/L, ≥1750 g/L, or ≥2000 g/L chromatography material. In some embodiments, the load density of the composition is between any of about 20 g/L and about 2000 g/L, about 50 g/L and about 2000 g/L, about 100 g/L and about 2000 g/L, about 200 g/L and about 2000 g/L, about 250 g/L and about 2000 g/L, about 300 g/L and about 2000 g/L, about 400 g/L and about 2000 g/L, about 500 g/L and about 2000 g/L, about 600 g/L and about 2000 g/L, about 700 g/L and about 2000 g/L, about 750 g/L and about 2000 g/L, about 800 g/L and about 2000 g/L, about 900 g/L and about 2000 g/L, about 1000 g/L and about 2000 g/L, about 1250 g/L and about 2000 g/L, or about 1500 g/L and about 2000 g/L chromatography material. In some embodiments, the load density of the composition is between any of about 20 g/L and about 1750 g/L, about 20 g/L and about 1500 g/L, about 20 g/L and about 1250 g/L, about 20 g/L and about 1200 g/L, or about 20 g/L and about 1100 g/L chromatography material.

In some embodiments, the load density of the composition exceeds the dynamic binding capacity of the chromatography material for the polypeptide. In some embodiments, the load density of the composition exceeds the dynamic binding capacity of the chromatography material for the polypeptide by about any of 2× (2-fold), 3×, 4×, 5×, 6×, 7×, 8×, 9×, 10×, 11×, 12×, 13×, 14×, 15×, 20×, 25×, 30×, 40×, 50×, 60×, 70×, 75×, 80×, 90×, or 100×. In some embodiments, the load density of the composition exceeds the dynamic binding capacity of the chromatography material for the polypeptide by between 2× and 100×, 5× and 100×, 10× and 100×, 20× and 100×, 25× and 100×, 30× and 100×, 40× and 100×, 50×, and 100×, 60× and 100×, 70× and 100×, 75× and 100×, 80× and 100×, or 90× and 100×. In some embodiments, the load density of the composition exceeds the dynamic binding capacity of the chromatography material for the polypeptide by between 2× and 100×, 2× and 75×, 2× and 50×, 2× and 25×, 2× and 20×, 2× and 15×, 2× and 14×, 2× and 13×, 2× and 12×, 2× and 11×, 2× and 10×, 2× and 9×, 2× and 8×, 2× and 7×, 2× and 6×, 2× and 5×, 2× and 4×, or 2× and 3×.

In some aspects, the invention provides methods for purifying a polypeptide from a composition comprising the polypeptide and an impurity using an ion exchange chromatography material wherein the ion exchange chromatography material is suitable for reuse, the method comprising the steps of a) equilibrating an ion exchange chromatography material with an equilibration buffer; b) loading the composition on the ion exchange chromatography material, wherein the load density exceeds the dynamic binding capacity of the ion exchange chromatography for the polypeptide; c) collecting fractions comprising the polypeptide; d) washing the ion exchange chromatography material with equilibration buffer; e) passing a first regeneration buffer through the ion exchange chromatography material, wherein the regeneration buffer comprises sodium acetate; f) passing a second regeneration buffer through the ion exchange chromatography material, wherein the second regeneration buffer comprises sodium acetate passing a regeneration buffer through the ion exchange chromatography material, wherein the regeneration buffer comprises sodium acetate; h) passing an acid wash through the ion exchange chromatography material; i) passing a sanitization buffer through the ion exchange chromatography material, wherein the sanitization buffer comprises about 0.5 N NaOH; and j) storing the ion exchange chromatography material in a storage buffer, wherein the storage buffer comprises about 0.1 N NaOH. In some embodiments, the ion exchange chromatography material is a cation exchange chromatography material. In some embodiments, the ion exchange chromatography material is an anion exchange chromatography material. In some embodiments, the ion exchange chromatography material is a mixed mode anion exchange chromatography material. In some embodiments, the ion exchange chromatography material is a Capto™ Adhere chromatography material.

In some embodiments, the equilibration buffer comprises about 50 mM to about 100 mM, about 55 mM to about 100 mM, about 60 mM to about 100 mM, about 65 mM to about 100 mM, about 70 mM to about 100 mM, about 75 mM to about 100 mM, about 80 mM to about 100 mM, about 85 mM to about 100 mM, about 90 mM to about 100 mM, about 95 mM to about 100 mM, about 50 mM to about 95 mM, about 50 mM to about 90 mM, about 50 mM to about 85 mM, about 50 mM to about 80 mM, about 50 mM to about 75 mM, about 50 mM to about 70 mM, about 50 mM to about 65 mM, about 50 mM to about 60 mM, or about 50 mM to about 55 mM NaOAc. In some embodiments, the equilibration buffer comprises about any of 50 mM, 55 mM, 60 mM, 65 mM, 70 mM, 75 mM, 80 mM, 85 mM, 90 mM, 95 mM, or 100 mM NaOAc. In some embodiments, the equilibration buffer comprises about 85 mM NaOAc. In some embodiments, the equilibration buffer comprises about 20 mM to about 80 mM, about 30 mM to about 80 mM, about 40 mM to about 80 mM, about 50 mM to about 80 mM, about 60 mM to about 80 mM, about 70 mM to about 80 mM, about 20 mM to about 70 mM, about 20 mM to about 60 mM, about 20 mM to about 50 mM, about 20 mM to about 40 mM, or about 20 mM to about 30 mM Tris. In some embodiments, the equilibration buffer comprises about any of 25 mM, 30 mM, 35 mM, 40 mM, 45 mM, 50 mM, 55 mM, 60 mM, 65 mM, 70 mM, or 70 mM Tris. In some embodiments, the equilibration buffer comprises about 50 mM Tris. In some embodiments, the equilibration buffer comprises about 50 mM Tris and 85 mM NaOAc.

In some embodiments, the pH of the equilibration buffer is about 6.0 to about 10.0, about 7.0 to about 10.0, about 8.0 to about 10.0, about 9.0 to about 10.0, about 9.0 to about 9.0, about 6.0 to about 8.0, or about 6.0 to about 7.0. In some embodiments, the pH of the equilibration buffer is about any of 6.0, 6.5, 7.0, 7.5, 8.0, 8.5, 9.0, 9.5 or 10.0. In some embodiments, the pH of the equilibration buffer is about 8.0. In some embodiments, the equilibration buffer comprises about 50 mM Tris and 85 mM NaOAc, about pH 8.0.

In some embodiments, about 3 to about 10, about 3 to about 9, about 3 to about 8, about 3 to about 7, about 3 to about 6, about 3 to about 5, about 3 to about 4, about 4 to about 10, about 5 to about 10, about 6 to about 10, about 7 to about 10, about 8 to about 10, about 9 to about 10, about 3 to about 8, about 4 to about 7, or about 5 to about 6 material volumes (e.g., column volumes) of equilibration buffer are passed through the ion exchange chromatography material prior to loading the composition. In some embodiments, about any of 3, 4, 5, 6, 7, 8, 9, or 10 material volumes (e.g., column volumes) of equilibration buffer are passed through the ion exchange chromatography material prior to loading the composition. In some embodiments, about 5 material volumes (e.g., column volumes) of equilibration buffer are passed through the ion exchange chromatography material prior to loading the composition. In some embodiments, the ion exchange material is washed with equilibration buffer after loading the composition until a sufficient amount of polypeptide is collected in the fractions. In some embodiments, about any of 5, 6, 7, 8, 9, 10, 11, 12, 13, 14, or 15 material volumes (e.g., column volumes) of equilibration buffer are passed through the ion exchange chromatography material after loading the composition. In some embodiments, about 10 material volumes (e.g., column volumes) of equilibration buffer are passed through the ion exchange chromatography material after loading the composition.

In some embodiments, the first regeneration buffer and/or the second regeneration buffer comprises about 50 to about 600 mM NaOAc. In some embodiments, the first regeneration buffer and/or the second regeneration buffer comprises NaOAc at a concentration range of about any of 25 mM to 600 mM, 30 mM to 600 mM, 40 mM to 600 mM, 50 mM to 600 mM, 75 mM to 600 mM, 100 mM to 600 mM, 125 mM to 600 mM, 150 mM to 600 mM, 175 mM to 600 mM, 200 mM to 600 mM, 225 mM to 600 mM, 250 mM to 600 mM, 275 mM to 600 mM, 300 mM to 600 mM, 325 mM to 600 mM, 350 mM to 600 mM, 375 mM to 600 mM, 400 mM to 600 mM, 450 mM to 600 mM, 500 mM to 600 mM, 550 mM to 600 mM, 25 mM to 550 mM, 25 mM to 500 mM, 25 mM to 475 mM, 25 mM to 450 mM, 25 mM to 425 mM, 25 mM to 400 mM, 25 mM to 375 mM, 25 mM to 550 mM, 25 mM to 325 mM, 25 mM to 300 mM, 25 mM to 275 mM, 25 mM to 250 mM, 25 mM to 225 mM, 25 mM to 200 mM, 25 mM to 175 mM, 25 mM to 150 mM, 25 mM to 125 mM, 25 mM to 100 mM, 25 mM to 90 mM, 25 mM to 80 mM, 25 mM to 70 mM, 25 mM to 60 mM, 25 mM to 50 mM, 25 mM to 40 mM, or 25 mM to 30 mM. In some embodiments, the first regeneration buffer comprises about any of 10, 20, 30, 40, 50, 60, 70, 80, 90, 100, 150, 200, 250, 300, 350, 400, 450, 500, 550, or 600 mM NaOAc. In some embodiments, the regeneration buffer comprises about 350 mM NaOAc.

In some embodiments, the pH of the first regeneration buffer and/or the second regeneration buffer is about pH 2.0 to about pH 7.0. In some embodiments, the pH of the first regeneration buffer and/or the second regeneration buffer is about pH 2.0 to about pH 6.5, about pH 2.0 to about pH 6.0, about pH 2.0 to about pH 5.5, about pH 2.0 to about pH 5.0, about pH 2.0 to about pH 4.5, about pH 2.0 to about pH 4.0, about pH 2.0 to about pH 3.5, about pH 2.0 to about pH 3.0, about pH 2.0 to about pH 2.5, about pH 2.5 to about pH 7.0, about pH 3.0 to about pH 7.0, about pH 3.5 to about pH 7.0, about pH 4.0 to about pH 7.0, about pH 4.5 to about pH 7.0, about pH 5.0 to about pH 7.0, about pH 5.5 to about pH 7.0, about pH 6.0 to about pH 7.0, or about pH 6.5 to about pH 7.0. In some embodiments, the regeneration buffer is about pH 2.0, about pH 8.0, about pH 2.5, about pH 3.0, about pH 3.5, about pH14.0, about pH 4.5, about pH 5.0, about pH 5.5, about pH 6.0, about pH 6.5, or about pH 7.0.

In some embodiments, the first regeneration buffer is about 40 mM NaOAc and about pH 4.0, and the second regeneration buffer is about 350 mM NaOAc and about pH 4.0. In some embodiments, the first regeneration buffer is about 40 mM NaOAc and about pH 5.0, and the second regeneration buffer is about 350 mM NaOAc and about pH 5.0. In some embodiments, the first regeneration buffer is about 350 mM NaOAc and about pH 4.0, and the second regeneration buffer is about 40 mM NaOAc and about pH 4.0. In some embodiments, the first regeneration buffer is about 350 mM NaOAc and about pH 5.0, and the second regeneration buffer is about 40 mM NaOAc and about pH 5.0.

In some embodiments, about 3 to about 12, about 3 to about 11, about 3 to about 10, about 3 to about 9, about 3 to about 8, about 3 to about 7, about 3 to about 6, about 3 to about 5, about 3 to about 4, about 4 to about 12, about 5 to about 12, about 6 to about 12, about 7 to about 12, about 8 to about 12, about 9 to about 12, about 10 to about 12, about 11 to about 12, about 5 to about 12, about 5 to about 10, about 8 to about 12, or about 9 to about 11 material volumes (e.g., column volumes) of the first regeneration buffer and/or the second regeneration buffer are passed through the ion exchange chromatography material. In some embodiments, about any of 3, 4, 5, 6, 7, 8, 9, 10, 11, or 12 material volumes (e.g., column volumes) of the first regeneration buffer and/or the second regeneration buffer are passed through the ion exchange chromatography material. In some embodiments, about 10 material volumes (e.g., column volumes) of first regeneration buffer and/or the second regeneration buffer are passed through the ion exchange chromatography material.

In some embodiments, the acid wash comprises NaOAc. In some embodiments, the acid wash comprises phosphoric acid. In some embodiments, the acid wash comprises NaOAc and phosphoric acid. In some embodiments, the concentration of NaOAc in the acid wash is more than about any of 0.1 M to less than about 0.5 M. In some embodiments, the concentration of NaOAc in the acid wash is any of about 0.100 M, 0.120M, 0.130M, 0.140M, 0.150M, 0.160M, 0.161M, 0.162M, 0.163M, 0.164M, 0.167M, 0.168M, 0.169M, 0.170M, 0.180M, 0.190M, 0.200M, 0.250M, 0.300M, 0.350M, 0.400M, 0.450M, or 0.500M. In some embodiments, the concentration of phosphoric acid in the acid wash is any of about 0.10 M, 0.15M, 0.20M, 0.25M, 0.30M, 0.35M, 0.40M, 0.45M, 0.50M, 0.55M, or 0.60M. In some embodiments, the acid wash comprises about 0.167M NaOAc and about 0.3M phosphoric acid.

In some embodiments, about 1 to about 5, about 1 to about 4, about 1 to about 3, about 1 to about 2, about 2 to about 5, about 2 to about 4, about 2 to about 3, about 3 to about 5, about 3 to about 4, about 4 to about 5 material volumes (e.g., column volumes) of acid wash are passed through the ion exchange chromatography material. In some embodiments, about any of 1, 2, 3, 4, or 5 material volumes (e.g., column volumes) of acid wash are passed through the ion exchange chromatography material. In some embodiments, about 3 material volumes (e.g., column volumes) of acid wash are passed through the ion exchange chromatography material.

In some embodiments, the sanitization buffer comprises about 0.1 N to about 1 N NaOH In some embodiments, the sanitization buffer comprises about 0.5 N NaOH. In some embodiments, the sanitization buffer comprises about 0.5 M to about 1.5 M NaCl. In some embodiments, the sanitization buffer comprises about 1.0 M NaCl. In some embodiments, the sanitization buffer comprises about 0.5 N NaOH and about 1M NaCl. In some embodiments, about 1 to about 10 material volumes of sanitization buffer are passed through the ion exchange chromatography material. In some embodiments, about 4 material volumes of sanitization buffer are passed through the ion exchange chromatography material.

In some embodiments, the storage buffer comprises about 0.05 N to about 0.5 N NaOH.

In some embodiments, the sanitization buffer comprises about 0.1 N NaOH. In some embodiments, the ion exchange chromatography material is stored in about 1 to 5 material volumes of storage buffer. In some embodiments, the ion exchange chromatography material is stored in about 3 material volumes of storage buffer.

In some embodiments, the load density of the composition is ≤1000 g/L chromatography material. In some embodiments, the load density of the composition is any of about ≤100 g/L, ≤900 g/L, ≤800 g/L, ≤750 g/L, ≤700 g/L, ≤600 g/L, ≤500 g/L, ≤400 g/L, ≤300 g/L, ≤250 g/L, ≤200 g/L, ≤100 g/L chromatography material. In some embodiments, the load density of the composition is between any of about 20 g/L and about 1000 g/L, about 50 g/L and about 1000 g/L, about 100 g/L and about 1000 g/L, about 200 g/L and about 1000 g/L, about 250 g/L and about 1000 g/L, about 300 g/L and about 1000 g/L, about 400 g/L and about 1000 g/L, about 500 g/L and about 1000 g/L, about 600 g/L and about 1000 g/L, about 700 g/L and about 1000 g/L, about 750 g/L and about 1000 g/L, about 800 g/L and about 1000 g/L, or about 900 g/L and about 1000 g/L, chromatography material. In some embodiments, the load density of the composition is between any of about 20 g/L and about 900 g/L, about 20 g/L and about 800 g/L, about 20 g/L and about 750 g/L, about 20 g/L and about 700 g/L, about 20 g/L and about 600 g/L, about 20 g/L and about 500 g/L, about 20 g/L and about 400 g/L, about 20 g/L and about 300 g/L, about 20 g/L and about 250 g/L, about 20 g/L and about 200 g/L, about 20 g/L and about 100 g/L, about 20 g/L and about 75 g/L, or about 20 g/L and about 50 g/L, chromatography material.

In some embodiments, the load density of the composition is ≥20 g/L chromatography material. In some embodiments, the load density of the composition is any of about ≥20 g/L, ≥50 g/L, ≥75 g/L, ≥100 g/L, ≥200 g/L, ≥250 g/L, ≥300 g/L, ≥400 g/L, ≥500 g/L, ≥600 g/L, ≥700 g/L, ≥750 g/L, ≥800 g/L, ≥900 g/L, ≥1000 g/L, ≥1200 g/L, ≥1400 g/L, ≥1500 g/L, ≥1750 g/L, or ≥2000 g/L chromatography material. In some embodiments, the load density of the composition is between any of about 20 g/L and about 2000 g/L, about 50 g/L and about 2000 g/L, about 100 g/L and about 2000 g/L, about 200 g/L and about 2000 g/L, about 250 g/L and about 2000 g/L, about 300 g/L and about 2000 g/L, about 400 g/L and about 2000 g/L, about 500 g/L and about 2000 g/L, about 600 g/L and about 2000 g/L, about 700 g/L and about 2000 g/L, about 750 g/L and about 2000 g/L, about 800 g/L and about 2000 g/L, about 900 g/L and about 2000 g/L, about 1000 g/L and about 2000 g/L, about 1250 g/L and about 2000 g/L, or about 1500 g/L and about 2000 g/L chromatography material. In some embodiments, the load density of the composition is between any of about 20 g/L and about 1750 g/L, about 20 g/L and about 1500 g/L, about 20 g/L and about 1250 g/L, about 20 g/L and about 1200 g/L, or about 20 g/L and about 1100 g/L chromatography material.

In some embodiments, the load density of the composition exceeds the dynamic binding capacity of the chromatography material for the polypeptide. In some embodiments, the load density of the composition exceeds the dynamic binding capacity of the chromatography material for the polypeptide by about any of 2× (2-fold), 3×, 4×, 5×, 6×, 7×, 8×, 9×, 10×, 11×, 12×, 13×, 14×, 15×, 20×, 25×, 30×, 40×, 50×, 60×, 70×, 75×, 80×, 90×, or 100×. In some embodiments, the load density of the composition exceeds the dynamic binding capacity of the chromatography material for the polypeptide by between 2× and 100×, 5× and 100×, 10× and 100×, 20× and 100×, 25× and 100×, 30× and 100×, 40× and 100×, 50× and 100×, 60× and 100×, 70×, and 100×, 75× and 100×, 80× and 100×, or 90× and 100×. In some embodiments, the load density of the composition exceeds the dynamic binding capacity of the chromatography material for the polypeptide by between 2× and 100×, 2× and 75×, 2× and 50×, 2× and 25×, 2× and 20×, 2× and 15×, 2× and 14×, 2× and 13×, 2× and 12×, 2× and 11×, 2× and 10×, 2× and 9×, 2× and 8×, 2× and 7×, 2× and 6×, 2× and 5×, 2× and 4×, or 2× and 3×.

In some aspects, the invention provides methods for purifying a polypeptide from a composition comprising the polypeptide and an impurity using an ion exchange chromatography material wherein the ion exchange chromatography material is suitable for reuse, the method comprising the steps of a) equilibrating an ion exchange chromatography material with an equilibration buffer, wherein the equilibration buffer comprises about 40 mM NaOAc; b) loading the composition on the ion exchange chromatography material, wherein the load density is ≤about 1000 g/L; c) collecting fractions comprising the polypeptide; d) washing the ion exchange chromatography material with equilibration buffer; e) passing a regeneration buffer through the ion exchange chromatography material, wherein the regeneration buffer comprises about 350 mM NaOAc at about pH 8.3; f) passing of sanitization buffer through the ion exchange chromatography material, wherein the sanitization buffer comprises about 0.5 N NaOH; and g) storing the ion exchange chromatography material in a storage buffer, wherein the storage buffer comprises about 0.1 N NaOH. In some aspects, the invention provides methods for purifying a polypeptide from a composition comprising the polypeptide and an impurity using an ion exchange chromatography material wherein the ion exchange chromatography material is suitable for reuse, the method comprising the steps of a) equilibrating an ion exchange chromatography material with about 4 material volumes of an equilibration buffer, wherein the equilibration buffer comprises about 40 mM NaOAc; b) loading the composition on the ion exchange chromatography material, wherein the load density is ≤about 1000 g/L; c) collecting fractions comprising the polypeptide; d) washing the ion exchange chromatography material with equilibration buffer; e) passing about 10 material volumes of a regeneration buffer through the ion exchange chromatography material, wherein the regeneration buffer comprises about 350 mM NaOAc at about pH 8.3; f) passing about 10 material volumes of sanitization buffer through the ion exchange chromatography material, wherein the sanitization buffer comprises about 0.5 N NaOH; and g) storing the ion exchange chromatography material in about 4 material volumes of a storage buffer, wherein the storage buffer comprises about 0.1 N NaOH In some embodiments, the load density of the composition is any of about ≤1000 g/L, ≤900 g/L, ≤800 g/L, ≤750 g/L, ≤700 g/L, ≤600 g/L, ≤500 g/L, ≤400 g/L, ≤300 g/L, ≤250 g/L, ≤200 g/L, ≤100 g/L chromatography material.

In some aspects, the invention provides methods for purifying a polypeptide from a composition comprising the polypeptide and an impurity using a cation exchange chromatography material wherein the cation exchange chromatography material is suitable for reuse, the method comprising the steps of a) equilibrating a cation exchange chromatography material with an equilibration buffer, wherein the equilibration buffer comprises about 40 mM NaOAc; b) loading the composition on the cation exchange chromatography material, wherein the load density is ≤about 1000 g/L; c) collecting fractions comprising the polypeptide; d) washing the cation exchange chromatography material with equilibration buffer; e) passing a regeneration buffer through the cation exchange chromatography material, wherein the regeneration buffer comprises about 350 mM NaOAc at about pH 8.3; f) passing of sanitization buffer through the cation exchange chromatography material, wherein the sanitization buffer comprises about 0.5 N NaOH; and g) storing the cation exchange chromatography material in a storage buffer, wherein the storage buffer comprises about 0.1 N NaOH. In some aspects, the invention provides methods for purifying a polypeptide from a composition comprising the polypeptide and an impurity using a cation exchange chromatography material wherein the cation exchange chromatography material is suitable for reuse, the method comprising the steps of a) equilibrating a cation exchange chromatography material with about 4 material volumes of an equilibration buffer, wherein the equilibration buffer comprises about 40 mM NaOAc; b) loading the composition on the cation exchange chromatography material, wherein the load density is ≤about 1000 g/L; c) collecting fractions comprising the polypeptide; d) washing the cation exchange chromatography material with equilibration buffer; e) passing about 10 material volumes of a regeneration buffer through the cation exchange chromatography material, wherein the regeneration buffer comprises about 350 mM NaOAc at about pH 8.3; f) passing about 10 material volumes of sanitization buffer through the cation exchange chromatography material, wherein the sanitization buffer comprises about 0.5 N NaOH; and g) storing the cation exchange chromatography material in about 4 material volumes of a storage buffer, wherein the storage buffer comprises about 0.1 N NaOH. In some embodiments, the load density of the composition is any of about ≤1000 g/L, ≤900 g/L, ≤800 g/L, ≤750 g/L, ≤700 g/L, ≤600 g/L, ≤500 g/L, ≤400 g/L, ≤300 g/L, ≤250 g/L, ≤200 g/L, ≤100 g/L chromatography material.

In some aspects, the invention provides methods for purifying a polypeptide from a composition comprising the polypeptide and an impurity using an anion exchange chromatography material wherein the anion exchange chromatography material is suitable for reuse, the method comprising the steps of a) equilibrating an anion exchange chromatography material with an equilibration buffer, wherein the equilibration buffer comprises about 40 mM NaOAc; b) loading the composition on the anion exchange chromatography material, wherein the load density is ≤about 1000 g/L; c) collecting fractions comprising the polypeptide; d) washing the anion exchange chromatography material with equilibration buffer; e) passing a regeneration buffer through the anion exchange chromatography material, wherein the regeneration buffer comprises about 350 mM NaOAc at about pH 8.3; f) passing of sanitization buffer through the anion exchange chromatography material, wherein the sanitization buffer comprises about 0.5 N NaOH; and g) storing the anion exchange chromatography material in a storage buffer, wherein the storage buffer comprises about 0.1 N NaOH. In some aspects, the invention provides methods for purifying a polypeptide from a composition comprising the polypeptide and an impurity using an anion exchange chromatography material wherein the anion exchange chromatography material is suitable for reuse, the method comprising the steps of a) equilibrating a anion exchange chromatography material with about 4 material volumes of an equilibration buffer, wherein the equilibration buffer comprises about 40 mM NaOAc; b) loading the composition on the anion exchange chromatography material, wherein the load density is ≤about 1000 g/L; c) collecting fractions comprising the polypeptide; d) washing the anion exchange chromatography material with equilibration buffer; e) passing about 10 material volumes of a regeneration buffer through the anion exchange chromatography material, wherein the regeneration buffer comprises about 350 mM NaOAc at about pH 8.3; f) passing about 10 material volumes of sanitization buffer through the anion exchange chromatography material, wherein the sanitization buffer comprises about 0.5 N NaOH; and g) storing the anion exchange chromatography material in about 4 material volumes of a storage buffer, wherein the storage buffer comprises about 0.1 N NaOH. In some embodiments, the load density of the composition is any of about ≤1000 g/L, ≤900 g/L, ≤800 g/L, ≤750 g/L, ≤700 g/L, ≤600 g/L, ≤500 g/L, ≤400 g/L, ≤300 g/L, ≤250 g/L, ≤200 g/L, ≤100 g/L chromatography material.

In some aspects, the invention provides methods for purifying a polypeptide from a composition comprising the polypeptide and an impurity using an ion exchange chromatography material wherein the ion exchange chromatography material is suitable for reuse, the method comprising the steps of a) equilibrating an ion exchange chromatography material with an equilibration buffer, wherein the equilibration buffer comprises about 40 mM NaOAc; b) loading the composition on the ion exchange chromatography material, wherein the load density is ≥about 20 g/L; c) collecting fractions comprising the polypeptide; d) washing the ion exchange chromatography material with equilibration buffer; e) passing a regeneration buffer through the ion exchange chromatography material, wherein the regeneration buffer comprises about 350 mM NaOAc at about pH 8.3; f) passing of sanitization buffer through the ion exchange chromatography material, wherein the sanitization buffer comprises about 0.5 N NaOH; and g) storing the ion exchange chromatography material in a storage buffer, wherein the storage buffer comprises about 0.1 N NaOH. In some aspects, the invention provides methods for purifying a polypeptide from a composition comprising the polypeptide and an impurity using an ion exchange chromatography material wherein the ion exchange chromatography material is suitable for reuse, the method comprising the steps of a) equilibrating an ion exchange chromatography material with about 4 material volumes of an equilibration buffer, wherein the equilibration buffer comprises about 40 mM NaOAc; b) loading the composition on the ion exchange chromatography material, wherein the load density is ≥about 20 g/L; c) collecting fractions comprising the polypeptide; d) washing the ion exchange chromatography material with equilibration buffer; e) passing about 10 material volumes of a regeneration buffer through the ion exchange chromatography material, wherein the regeneration buffer comprises about 350 mM NaOAc at about pH 8.3; f) passing about 10 material volumes of sanitization buffer through the ion exchange chromatography material, wherein the sanitization buffer comprises about 0.5 N NaOH; and g) storing the ion exchange chromatography material in about 4 material volumes of a storage buffer, wherein the storage buffer comprises about 0.1 N NaOH. In some embodiments, the load density of the composition is any of about ≥20 g/L, ≥50 g/L, ≥70 g/L, ≥100 g/L, ≥200 g/L, ≥250 g/L, ≥300 g/L, ≥400 g/L, ≥500 g/L, ≥600 g/L, ≥700 g/L, ≥750 g/L, ≥800 g/L, ≥900 g/L, ≥1000 g/L, ≥1200 g/L, ≥1400 g/L, ≥1500 g/L, ≥1750 g/L, or ≥2000 g/L chromatography material.

In some aspects, the invention provides methods for purifying a polypeptide from a composition comprising the polypeptide and an impurity using a cation exchange chromatography material wherein the cation exchange chromatography material is suitable for reuse, the method comprising the steps of a) equilibrating a cation exchange chromatography material with an equilibration buffer, wherein the equilibration buffer comprises about 40 mM NaOAc; b) loading the composition on the cation exchange chromatography material, wherein the load density is ≥about 20 g/L; c) collecting fractions comprising the polypeptide; d) washing the cation exchange chromatography material with equilibration buffer; e) passing a regeneration buffer through the cation exchange chromatography material, wherein the regeneration buffer comprises about 350 mM NaOAc at about pH 8.3; f) passing of sanitization buffer through the cation exchange chromatography material, wherein the sanitization buffer comprises about 0.5 N NaOH; and g) storing the cation exchange chromatography material in a storage buffer, wherein the storage buffer comprises about 0.1 N NaOH. In some aspects, the invention provides methods for purifying a polypeptide from a composition comprising the polypeptide and an impurity using a cation exchange chromatography material wherein the cation exchange chromatography material is suitable for reuse, the method comprising the steps of a) equilibrating a cation exchange chromatography material with about 4 material volumes of an equilibration buffer, wherein the equilibration buffer comprises about 40 mM NaOAc; b) loading the composition on the cation exchange chromatography material, wherein the load density is 2 about 20 g/L; c) collecting fractions comprising the polypeptide; d) washing the cation exchange chromatography material with equilibration buffer; e) passing about 10 material volumes of a regeneration buffer through the cation exchange chromatography material, wherein the regeneration buffer comprises about 350 mM NaOAc at about pH 8.3; f) passing about 10 material volumes of sanitization buffer through the cation exchange chromatography material, wherein the sanitization buffer comprises about 0.5 N NaOH; and g) storing the cation exchange chromatography material in about 4 material volumes of a storage buffer, wherein the storage buffer comprises about 0.1 N NaOH. In some embodiments, the load density of the composition is any of about ≥20 g/L, ≥50 g/L, ≥70 g/L, ≥100 g/L, ≥200 g/L, ≥250 g/L, ≥300 g/L, ≥400 g/L, ≥500 g/L, ≥600 g/L, ≥700 g/L, ≥750 g/L, ≥800 g/L, ≥900 g/L, ≥1000 g/L, ≥1200 g/L, ≥1400 g/L, ≥1500 g/L, ≥1750 g/L, or ≥22000 g/L chromatography material.

In some aspects, the invention provides methods for purifying a polypeptide from a composition comprising the polypeptide and an impurity using an anion exchange chromatography material wherein the anion exchange chromatography material is suitable for reuse, the method comprising the steps of a) equilibrating an anion exchange chromatography material with an equilibration buffer, wherein the equilibration buffer comprises about 40 mM NaOAc; b) loading the composition on the anion exchange chromatography material, wherein the load density is ≥about 20 g/L; c) collecting fractions comprising the polypeptide; d) washing the anion exchange chromatography material with equilibration buffer; e) passing a regeneration buffer through the anion exchange chromatography material, wherein the regeneration buffer comprises about 350 mM NaOAc at about pH 8.3; f) passing of sanitization buffer through the anion exchange chromatography material, wherein the sanitization buffer comprises about 0.5 N NaOH; and g) storing the anion exchange chromatography material in a storage buffer, wherein the storage buffer comprises about 0.1 N NaOH. In some aspects, the invention provides methods for purifying a polypeptide from a composition comprising the polypeptide and an impurity using an anion exchange chromatography material wherein the anion exchange chromatography material is suitable for reuse, the method comprising the steps of a) equilibrating a anion exchange chromatography material with about 4 material volumes of an equilibration buffer, wherein the equilibration buffer comprises about 40 mM NaOAc; b) loading the composition on the anion exchange chromatography material, wherein the load density is ≥about 20 g/L; c) collecting fractions comprising the polypeptide; d) washing the anion exchange chromatography material with equilibration buffer; e) passing about 10 material volumes of a regeneration buffer through the anion exchange chromatography material, wherein the regeneration buffer comprises about 350 mM NaOAc at about pH 8.3; f) passing about 10 material volumes of sanitization buffer through the anion exchange chromatography material, wherein the sanitization buffer comprises about 0.5 N NaOH; and g) storing the anion exchange chromatography material in about 4 material volumes of a storage buffer, wherein the storage buffer comprises about 0.1 N NaOH. In some embodiments, the load density of the composition is any of about ≥20 g/L, ≥50 g/L, ≥70 g/L, ≥100 g/L, ≥200 g/L, ≥250 g/L, ≥300 g/L, ≥400 g/L, ≥500 g/L, ≥600 g/L, ≥700 g/L, ≥750 g/L, ≥800 g/L, ≥900 g/L, ≥1000 g/L, ≥1200 g/L, ≥1400 g/L, ≥1500 g/L, ≥1750 g/L, or ≥2000 g/L chromatography material.

In some aspects, the invention provides methods for purifying a polypeptide from a composition comprising the polypeptide and an impurity using an ion exchange chromatography material wherein the ion exchange chromatography material is suitable for reuse, the method comprising the steps of a) equilibrating an ion exchange chromatography material with an equilibration buffer, wherein the equilibration buffer comprises about 40 mM NaOAc; b) loading the composition on the ion exchange chromatography material, wherein the load density exceeds the dynamic binding capacity of the ion exchange chromatography material for the polypeptide; c) collecting fractions comprising the polypeptide; d) washing the ion exchange chromatography material with equilibration buffer; e) passing a regeneration buffer through the ion exchange chromatography material, wherein the regeneration buffer comprises about 350 mM NaOAc at about pH 8.3; f) passing of sanitization buffer through the ion exchange chromatography material, wherein the sanitization buffer comprises about 0.5 N NaOH; and g) storing the ion exchange chromatography material in a storage buffer, wherein the storage buffer comprises about 0.1 N NaOH. In some aspects, the invention provides methods for purifying a polypeptide from a composition comprising the polypeptide and an impurity using an ion exchange chromatography material wherein the ion exchange chromatography material is suitable for reuse, the method comprising the steps of a) equilibrating an ion exchange chromatography material with about 4 material volumes of an equilibration buffer, wherein the equilibration buffer comprises about 40 mM NaOAc; b) loading the composition on the ion exchange chromatography material, wherein the load density exceeds the dynamic binding capacity of the ion exchange chromatography material for the polypeptide; c) collecting fractions comprising the polypeptide; d) washing the ion exchange chromatography material with equilibration buffer; e) passing about 10 material volumes of a regeneration buffer through the ion exchange chromatography material, wherein the regeneration buffer comprises about 350 mM NaOAc at about pH 8.3; f) passing about 10 material volumes of sanitization buffer through the ion exchange chromatography material, wherein the sanitization buffer comprises about 0.5 N NaOH; and g) storing the ion exchange chromatography material in about 4 material volumes of a storage buffer, wherein the storage buffer comprises about 0.1 N NaOH. In some embodiments, the load density of the composition exceeds the dynamic binding capacity of the chromatography material for the polypeptide by between 2× and 100×. In some embodiments, the load density of the composition exceeds the dynamic binding capacity of the chromatography material for the polypeptide by about 10×.

In some aspects, the invention provides methods for purifying a polypeptide from a composition comprising the polypeptide and an impurity using a cation exchange chromatography material wherein the cation exchange chromatography material is suitable for reuse, the method comprising the steps of a) equilibrating a cation exchange chromatography material with an equilibration buffer, wherein the equilibration buffer comprises about 40 mM NaOAc; b) loading the composition on the cation exchange chromatography material, wherein the load density exceeds the dynamic binding capacity of the cation exchange chromatography material for the polypeptide; c) collecting fractions comprising the polypeptide; d) washing the cation exchange chromatography material with equilibration buffer; e) passing a regeneration buffer through the cation exchange chromatography material, wherein the regeneration buffer comprises about 350 mM NaOAc at about pH 8.3; f) passing of sanitization buffer through the cation exchange chromatography material, wherein the sanitization buffer comprises about 0.5 N NaOH; and g) storing the cation exchange chromatography material in a storage buffer, wherein the storage buffer comprises about 0.1 N NaOH. In some aspects, the invention provides methods for purifying a polypeptide from a composition comprising the polypeptide and an impurity using a cation exchange chromatography material wherein the cation exchange chromatography material is suitable for reuse, the method comprising the steps of a) equilibrating a cation exchange chromatography material with about 4 material volumes of an equilibration buffer, wherein the equilibration buffer comprises about 40 mM NaOAc; b) loading the composition on the cation exchange chromatography material, wherein the load density exceeds the dynamic binding capacity of the cation exchange chromatography material for the polypeptide; c) collecting fractions comprising the polypeptide; d) washing the cation exchange chromatography material with equilibration buffer; c) passing about 10 material volumes of a regeneration buffer through the cation exchange chromatography material, wherein the regeneration buffer comprises about 350 mM NaOAc at about pH 8.3, f) passing about 10 material volumes of sanitization buffer through the cation exchange chromatography material, wherein the sanitization buffer comprises about 0.5 N NaOH; and g) storing the cation exchange chromatography material in about 4 material volumes of a storage buffer, wherein the storage buffer comprises about 0.1 N NaOH. In some embodiments, the load density of the composition exceeds the dynamic binding capacity of the chromatography material for the polypeptide by between 2× and 100×. In some embodiments, the load density of the composition exceeds the dynamic binding capacity of the chromatography material for the polypeptide by about 10×.

In some aspects, the invention provides methods for purifying a polypeptide from a composition comprising the polypeptide and an impurity using an anion exchange chromatography material wherein the anion exchange chromatography material is suitable for reuse, the method comprising the steps of a) equilibrating an anion exchange chromatography material with an equilibration buffer, wherein the equilibration buffer comprises about 40 mM NaOAc; b) loading the composition on the anion exchange chromatography material, wherein the load density exceeds the dynamic binding capacity of the anion exchange chromatography material for the polypeptide; c) collecting fractions comprising the polypeptide; d) washing the anion exchange chromatography material with equilibration buffer; e) passing a regeneration buffer through the anion exchange chromatography material, wherein the regeneration buffer comprises about 350 mM NaOAc at about pH 8.3; f) passing of sanitization buffer through the anion exchange chromatography material, wherein the sanitization buffer comprises about 0.5 N NaOH; and g) storing the anion exchange chromatography material in a storage buffer, wherein the storage buffer comprises about 0.1 N NaOH. In some aspects, the invention provides methods for purifying a polypeptide from a composition comprising the polypeptide and an impurity using an anion exchange chromatography material wherein the anion exchange chromatography material is suitable for reuse, the method comprising the steps of a) equilibrating a anion exchange chromatography material with about 4 material volumes of an equilibration buffer, wherein the equilibration buffer comprises about 40 mM NaOAc; b) loading the composition on the anion exchange chromatography material, wherein the load density exceeds the dynamic binding capacity of the ion exchange chromatography material for the polypeptide; c) collecting fractions comprising the polypeptide; d) washing the anion exchange chromatography material with equilibration buffer; e) passing about 10 material volumes of a regeneration buffer through the anion exchange chromatography material, wherein the regeneration buffer comprises about 350 mM NaOAc at about pH 8.3; f) passing about 10 material volumes of sanitization buffer through the anion exchange chromatography material, wherein the sanitization buffer comprises about 0.5 N NaOH; and g) storing the anion exchange chromatography material in about 4 material volumes of a storage buffer, wherein the storage buffer comprises about 0.1 N NaOH. In some embodiments, the load density of the composition exceeds the dynamic binding capacity of the chromatography material for the polypeptide by between 2× and 100×. In some embodiments, the load density of the composition exceeds the dynamic binding capacity of the chromatography material for the polypeptide by about 10×.

In some aspects, the invention provides methods for purifying a polypeptide from a composition comprising the polypeptide and an impurity using an ion exchange chromatography material wherein the ion exchange chromatography material is suitable for reuse, the method comprising the steps of a) equilibrating an ion exchange chromatography material with an equilibration buffer, wherein the equilibration buffer comprises about 40 mM NaOAc; b) loading the composition on the ion exchange chromatography material, wherein the load density is ≤about 1000 g/L; c) collecting fractions comprising the polypeptide; d) washing the ion exchange chromatography material with equilibration buffer; e) passing a regeneration buffer through the ion exchange chromatography material, wherein the regeneration buffer comprises about 25 mM Tris and about 85 mM NaOAc at about pH 8.0; f) passing of sanitization buffer through the ion exchange chromatography material, w % herein the sanitization buffer comprises about 0.5 N NaOH; and g) storing the ion exchange chromatography material in a storage buffer, wherein the storage buffer comprises about 0.1 N NaOH. In some aspects, the invention provides methods for purifying a polypeptide from a composition comprising the polypeptide and an impurity using an ion exchange chromatography material wherein the ion exchange chromatography material is suitable for reuse, the method comprising the steps of a) equilibrating an ion exchange chromatography material with about 4 material volumes of an equilibration buffer, wherein the equilibration buffer comprises about 40 mM NaOAc; b) loading the composition on the ion exchange chromatography material, wherein the load density is ≤about 1000 g/L, c) collecting fractions comprising the polypeptide, d) washing the ion exchange chromatography material with equilibration buffer; e) passing about 10 material volumes of a regeneration buffer through the ion exchange chromatography material, wherein the regeneration buffer comprises about 25 mM Tris and about 85 mM NaOAc at about pH 8.0; f) passing about 10 material volumes of sanitization buffer through the ion exchange chromatography material, wherein the sanitization buffer comprises about 0.5 N NaOH; and g) storing the ion exchange chromatography material in about 4 material volumes of a storage buffer, wherein the storage buffer comprises about 0.1 N NaOH. In some embodiments, the load density of the composition is any of about ≤1000 g/L, ≤900 g/L, ≤800 g/L, ≤750 g/L, ≤700 g/L, ≤600 g/L, ≤500 g/L, ≤400 g/L, ≤300 g/L, ≤250 g/L, ≤200 g/L, ≤100 g/L chromatography material.

In some aspects, the invention provides methods for purifying a polypeptide from a composition comprising the polypeptide and an impurity using a cation exchange chromatography material wherein the cation exchange chromatography material is suitable for reuse, the method comprising the steps of a) equilibrating a cation exchange chromatography material with an equilibration buffer, wherein the equilibration buffer comprises about 40 mM NaOAc; b) loading the composition on the cation exchange chromatography material, wherein the load density is ≤about 1000 g/L; c) collecting fractions comprising the polypeptide; d) washing the cation exchange chromatography material with equilibration buffer; e) passing a regeneration buffer through the cation exchange chromatography material, wherein the regeneration buffer comprises about 25 mM Tris and about 85 mM NaOAc at about pH 8.0; f) passing of sanitization buffer through the cation exchange chromatography material, wherein the sanitization buffer comprises about 0.5 N NaOH; and g) storing the cation exchange chromatography material in a storage buffer, wherein the storage buffer comprises about 0.1 N NaOH. In some aspects, the invention provides methods for purifying a polypeptide from a composition comprising the polypeptide and an impurity using a cation exchange chromatography material wherein the cation exchange chromatography material is suitable for reuse, the method comprising the steps of a) equilibrating a cation exchange chromatography material with about 4 material volumes of an equilibration buffer, wherein the equilibration buffer comprises about 40 mM NaOAc; b) loading the composition on the cation exchange chromatography material, wherein the load density is ≤about 1000 g/L; c) collecting fractions comprising the polypeptide; d) washing the cation exchange chromatography material with equilibration buffer; e) passing about 10 material volumes of a regeneration buffer through the cation exchange chromatography material, wherein the regeneration buffer comprises about 25 mM Tris and about 85 mM NaOAc at about pH 8.0; f) passing about 10 material volumes of sanitization buffer through the cation exchange chromatography material, wherein the sanitization buffer comprises about 0.5 N NaOH; and g) storing the cation exchange chromatography material in about 4 material volumes of a storage buffer, wherein the storage buffer comprises about 0.1 N NaOH. In some embodiments, the load density of the composition is any of about ≤1000 g/L, ≤900 g/L, ≤800 g/L, ≤750 g/L, ≤700 g/L, ≤600 g/L, ≤500 g/L, ≤400 g/L, ≤300 g/L, ≤250 g/L, ≤200 g/L, ≤100 g/L chromatography material.

In some aspects, the invention provides methods for purifying a polypeptide from a composition comprising the polypeptide and an impurity using an anion exchange chromatography material wherein the anion exchange chromatography material is suitable for reuse, the method comprising the steps of a) equilibrating an anion exchange chromatography material with an equilibration buffer, wherein the equilibration buffer comprises about 40 mM NaOAc; b) loading the composition on the anion exchange chromatography material, wherein the load density is ≤about 1000 g/L; c) collecting fractions comprising the polypeptide; d) washing the anion exchange chromatography material with equilibration buffer; e) passing a regeneration buffer through the anion exchange chromatography material, wherein the regeneration buffer comprises about 25 mM Tris and about 85 mM NaOAc at about pH 8.0; f) passing of sanitization buffer through the anion exchange chromatography material, wherein the sanitization buffer comprises about 0.5 N NaOH; and g) storing the anion exchange chromatography material in a storage buffer, wherein the storage buffer comprises about 0.1 N NaOH. In some aspects, the invention provides methods for purifying a polypeptide from a composition comprising the polypeptide and an impurity using an anion exchange chromatography material wherein the anion exchange chromatography material is suitable for reuse, the method comprising the steps of a) equilibrating an anion exchange chromatography material with about 4 material volumes of an equilibration buffer, wherein the equilibration buffer comprises about 40 mM NaOAc; b) loading the composition on the anion exchange chromatography material, wherein the load density is ≤about 1000 g/L; c) collecting fractions comprising the polypeptide; d) washing the anion exchange chromatography material with equilibration buffer; e) passing about 10 material volumes of a regeneration buffer through the anion exchange chromatography material, wherein the regeneration buffer comprises about 25 mM Tris and about 85 mM NaOAc at about pH 8.0; f) passing about 10 material volumes of sanitization buffer through the anion exchange chromatography material, wherein the sanitization buffer comprises about 0.5 N NaOH; and g) storing the anion exchange chromatography material in about 4 material volumes of a storage buffer, wherein the storage buffet comprises about 0.1 N NaOH. In some embodiments, the load density of the composition is any of about ≤1000 g/L, ≤900 g/L, ≤800 g/L, ≤750 g/L, ≤700 g/L, ≤600 g/L, ≤500 g/L, ≤400 g/L, ≤300 g/L, ≤250 g/L, ≤200 g/L, ≤100 g/L chromatography material.

In some aspects, the invention provides methods for purifying a polypeptide from a composition comprising the polypeptide and an impurity using an ion exchange chromatography material wherein the ion exchange chromatography material is suitable for reuse, the method comprising the steps of a) equilibrating an ion exchange chromatography material with an equilibration buffer, wherein the equilibration buffer comprises about 40 mM NaOAc; b) loading the composition on the ion exchange chromatography material, wherein the load density is ≥about 20 g/L, c) collecting fractions comprising the polypeptide; d) washing the ion exchange chromatography material with equilibration buffer; e) passing a regeneration buffer through the ion exchange chromatography material, wherein the regeneration buffer comprises about 25 mM Tris and about 85 mM NaOAc at about pH 8.0; f) passing of sanitization buffer through the ion exchange chromatography material, wherein the sanitization buffer comprises about 0.5 N NaOH; and g) storing the ion exchange chromatography material in a storage buffer, wherein the storage buffer comprises about 0.1 N NaOH. In some aspects, the invention provides methods for purifying a polypeptide from a composition comprising the polypeptide and an impurity using an ion exchange chromatography material wherein the ion exchange chromatography material is suitable for reuse, the method comprising the steps of a) equilibrating an ion exchange chromatography material with about 4 material volumes of an equilibration buffer, wherein the equilibration buffer comprises about 40 mM NaOAc; b) loading the composition on the ion exchange chromatography material, wherein the load density is ≥about 20 g/L; c) collecting fractions comprising the polypeptide; d) washing the ion exchange chromatography material with equilibration buffer; e) passing about 10 material volumes of a regeneration buffer through the ion exchange chromatography material, wherein the regeneration buffer comprises about 25 mM Tris and about 85 mM NaOAc at about pH 8.0; f) passing about 10 material volumes of sanitization buffer through the ion exchange chromatography material, wherein the sanitization buffer comprises about 0.5 N NaOH; and g) storing the ion exchange chromatography material in about 4 material volumes of a storage buffer, wherein the storage buffer comprises about 0.1 N NaOH. In some embodiments, the load density of the composition is any of about ≥20 g/L, ≥50 g/L, ≥70 g/L, ≥100 g/L, ≥200 g/L, ≥250 g/L, ≥300 g/L, ≥400 g/L, ≥500 g/L, ≥600 g/L, ≥700 g/L, ≥750 g/L, ≥800 g/L, ≥900 g/L, ≥1000 g/L, ≥1200 g/L, ≥1400 g/L, ≥1500 g/L, ≥1750 g/L, or ≥2000 g/L chromatography material.

In some aspects, the invention provides methods for purifying a polypeptide from a composition comprising the polypeptide and an impurity using a cation exchange chromatography material wherein the cation exchange chromatography material is suitable for reuse, the method comprising the steps of a) equilibrating a cation exchange chromatography material with an equilibration buffer, wherein the equilibration buffer comprises about 40 mM NaOAc; b) loading the composition on the cation exchange chromatography material, wherein the load density is ≥about 20 g/L; c) collecting fractions comprising the polypeptide; d) washing the cation exchange chromatography material with equilibration buffer; e) passing a regeneration buffer through the cation exchange chromatography material, wherein the regeneration buffer comprises about 25 mM Tris and about 85 mM NaOAc at about pH 8.0; f) passing of sanitization buffer through the cation exchange chromatography material, wherein the sanitization buffer comprises about 0.5 N NaOH; and g) storing the cation exchange chromatography material in a storage buffer, wherein the storage buffer comprises about 0.1 N NaOH. In some aspects, the invention provides methods for purifying a polypeptide from a composition comprising the polypeptide and an impurity using a cation exchange chromatography material wherein the cation exchange chromatography material is suitable for reuse, the method comprising the steps of a) equilibrating a cation exchange chromatography material with about 4 material volumes of an equilibration buffer, wherein the equilibration buffer comprises about 40 mM NaOAc; b) loading the composition on the cation exchange chromatography material, wherein the load density is ≥about 20 g/L; c) collecting fractions comprising the polypeptide; d) washing the cation exchange chromatography material with equilibration buffer; c) passing about 10 material volumes of a regeneration buffer through the cation exchange chromatography material, wherein the regeneration buffer comprises about 25 mM Tris and about 85 mM NaOAc at about pH 8.0; f) passing about 10 material volumes of sanitization buffer through the cation exchange chromatography material, wherein the sanitization buffer comprises about 0.5 N NaOH; and g) storing the cation exchange chromatography material in about 4 material volumes of a storage buffer, wherein the storage buffer comprises about 0.1 N NaOH. In some embodiments, the load density of the composition is any of about ≥20 g/L, ≥50 g/L, ≥70 g/L, ≥100 g/L, ≥200 g/L, ≥250 g/L, ≥300 g/L, ≥400 g/L, ≥500 g/L, ≥600 g/L, ≥700 g/L, ≥750 g/L, ≥800 g/L, ≥900 g/L, ≥1000 g/L, ≥1200 g/L, ≥1400 g/L, ≥1500 g/L, ≥1750 g/L, or ≥2000 g/L chromatography material.

In some aspects, the invention provides methods for purifying a polypeptide from a composition comprising the polypeptide and an impurity using an anion exchange chromatography material wherein the anion exchange chromatography material is suitable for reuse, the method comprising the steps of a) equilibrating an anion exchange chromatography material with an equilibration buffer, wherein the equilibration buffer comprises about 40 mM NaOAc; b) loading the composition on the anion exchange chromatography material, wherein the load density is ≥about 20 g/L; c) collecting fractions comprising the polypeptide; d) washing the anion exchange chromatography material with equilibration buffer; e) passing a regeneration buffer through the anion exchange chromatography material, wherein the regeneration buffer comprises about 25 mM Tris and about 85 mM NaOAc at about pH 8.0; f) passing of sanitization buffer through the anion exchange chromatography material, wherein the sanitization buffer comprises about 0.5 N NaOH; and g) storing the anion exchange chromatography material in a storage buffer, wherein the storage buffer comprises about 0.1 N NaOH. In some aspects, the invention provides methods for purifying a polypeptide from a composition comprising the polypeptide and an impurity using an anion exchange chromatography material wherein the anion exchange chromatography material is suitable for reuse, the method comprising the steps of a) equilibrating an anion exchange chromatography material with about 4 material volumes of an equilibration buffer, wherein the equilibration buffer comprises about 40 mM NaOAc; b) loading the composition on the anion exchange chromatography material, wherein the load density is ≥about 20 g/L; c) collecting fractions comprising the polypeptide; d) washing the anion exchange chromatography material with equilibration buffer; e) passing about 10 material volumes of a regeneration buffer through the anion exchange chromatography material, wherein the regeneration buffer comprises about 25 mM Tris and about 85 mM NaOAc at about pH 8.0; f) passing about 10 material volumes of sanitization buffer through the anion exchange chromatography material, wherein the sanitization buffer comprises about 0.5 N NaOH; and g) storing the anion exchange chromatography material in about 4 material volumes of a storage buffer, wherein the storage buffer comprises about 0.1 N NaOH. In some embodiments, the load density of the composition is any of about ≥20 g/L, ≥50 g/L, ≥70 g/L, ≥100 g/L, ≥200 g/L, ≥250 g/L, ≥300 g/L, ≥400 g/L, ≥500 g/L, ≥600 g/L, ≥700 g/L, ≥750 g/L, ≥200 g/L, ≥900 g/L, ≥1000 g/L, ≥1200 g/L, ≥1400 g/L, ≥1500 g/L, ≥1750 g/L, or ≥2000 g/L chromatography material.

In some aspects, the invention provides methods for purifying a polypeptide from a composition comprising the polypeptide and an impurity using an ion exchange chromatography material wherein the ion exchange chromatography material is suitable for reuse, the method comprising the steps of a) equilibrating an ion exchange chromatography material with an equilibration buffer, wherein the equilibration buffer comprises about 40 mM NaOAc; b) loading the composition on the ion exchange chromatography material, wherein the load density exceeds the dynamic binding capacity of the ion exchange chromatography material for the polypeptide; c) collecting fractions comprising the polypeptide, d) washing the ion exchange chromatography material with equilibration buffer; e) passing a regeneration buffer through the ion exchange chromatography material, wherein the regeneration buffer comprises about 25 mM Tris and about 85 mM NaOAc at about pH 8.0; f) passing of sanitization buffer through the ion exchange chromatography material, wherein the sanitization buffer comprises about 0.5 N NaOH; and g) storing the ion exchange chromatography material in a storage buffer, wherein the storage buffer comprises about 0.1 N NaOH. In some aspects, the invention provides methods for purifying a polypeptide from a composition comprising the polypeptide and an impurity using an ion exchange chromatography material wherein the ion exchange chromatography material is suitable for reuse, the method comprising the steps of a) equilibrating an ion exchange chromatography material with about 4 material volumes of an equilibration buffer, wherein the equilibration buffer comprises about 40 mM NaOAc; b) loading the composition on the ion exchange chromatography material, wherein the load density exceeds the dynamic binding capacity of the ion exchange chromatography material for the polypeptide; c) collecting fractions comprising the polypeptide; d) washing the ion exchange chromatography material with equilibration buffer; e) passing about 10 material volumes of a regeneration buffer through the ion exchange chromatography material, wherein the regeneration buffer comprises about 25 mM Tris and about 85 mM NaOAc at about pH 8.0; f) passing about 10 material volumes of sanitization buffer through the ion exchange chromatography material, wherein the sanitization buffer comprises about 0.5 N NaOH; and g) storing the ion exchange chromatography material in about 4 material volumes of a storage buffer, wherein the storage buffer comprises about 0.1 N NaOH. In some embodiments, the load density of the composition exceeds the dynamic binding capacity of the chromatography material for the polypeptide by between 2× and 100×. In some embodiments, the load density of the composition exceeds the dynamic binding capacity of the chromatography material for the polypeptide by about 10×.

In some aspects, the invention provides methods for purifying a polypeptide from a composition comprising the polypeptide and an impurity using a cation exchange chromatography material wherein the cation exchange chromatography material is suitable for reuse, the method comprising the steps of a) equilibrating a cation exchange chromatography material with an equilibration buffer, wherein the equilibration buffer comprises about 40 mM NaOAc; b) loading the composition on the cation exchange chromatography material, wherein the load density exceeds the dynamic binding capacity of the cation exchange chromatography material for the polypeptide; c) collecting fractions comprising the polypeptide; d) washing the cation exchange chromatography material with equilibration buffer; e) passing a regeneration buffer through the cation exchange chromatography material, wherein the regeneration buffer comprises about 25 mM Tris and about 85 mM NaOAc at about pH 8.0; f) passing of sanitization buffer through the cation exchange chromatography material, wherein the sanitization buffer comprises about 0.5 N NaOH; and g) storing the cation exchange chromatography material in a storage buffer, wherein the storage buffer comprises about 0.1 N NaOH. In some aspects, the invention provides methods for purifying a polypeptide from a composition comprising the polypeptide and an impurity using a cation exchange chromatography material wherein the cation exchange chromatography material is suitable for reuse, the method comprising the steps of a) equilibrating a cation exchange chromatography material with about 4 material volumes of an equilibration buffer, wherein the equilibration buffer comprises about 40 mM NaOAc; b) loading the composition on the cation exchange chromatography material, wherein the load density exceeds the dynamic binding capacity of the cation exchange chromatography material for the polypeptide; c) collecting fractions comprising the polypeptide; d) washing the cation exchange chromatography material with equilibration buffer; e) passing about 10 material volumes of a regeneration buffer through the cation exchange chromatography material, wherein the regeneration buffer comprises about 25 mM Tris and about 85 mM NaOAc at about pH 8.0; f) passing about 10 material volumes of sanitization buffer through the cation exchange chromatography material, wherein the sanitization buffer comprises about 0.5 N NaOH; and g) storing the cation exchange chromatography material in about 4 material volumes of a storage buffer, wherein the storage buffer comprises about 0.1 N NaOH. In some embodiments, the load density of the composition exceeds the dynamic binding capacity of the chromatography material for the polypeptide by between 2× and 100×. In some embodiments, the load density of the composition exceeds the dynamic binding capacity of the chromatography material for the polypeptide by about 10×.

In some aspects, the invention provides methods for purifying a polypeptide from a composition comprising the polypeptide and an impurity using an anion exchange chromatography material wherein the anion exchange chromatography material is suitable for reuse, the method comprising the steps of a) equilibrating an anion exchange chromatography material with an equilibration buffer, wherein the equilibration buffer comprises about 40 mM NaOAc; b) loading the composition on the anion exchange chromatography material, wherein the load density exceeds the dynamic binding capacity of the anion exchange chromatography material for the polypeptide; c) collecting fractions comprising the polypeptide; d) washing the anion exchange chromatography material with equilibration buffer; e) passing a regeneration buffet through the anion exchange chromatography material, wherein the regeneration buffer comprises about 25 mM Tris and about 85 mM NaOAc at about pH 8.0; f) passing of sanitization buffer through the anion exchange chromatography material, wherein the sanitization buffer comprises about 0.5 N NaOH; and g) storing the anion exchange chromatography material in a storage buffer, wherein the storage buffer comprises about 0.1 N NaOH. In some aspects, the invention provides methods for purifying a polypeptide from a composition comprising the polypeptide and an impurity using an anion exchange chromatography material wherein the anion exchange chromatography material is suitable for reuse, the method comprising the steps of a) equilibrating an anion exchange chromatography material with about 4 material volumes of an equilibration buffer, wherein the equilibration buffer comprises about 40 mM NaOAc; b) loading the composition on the anion exchange chromatography material, wherein the load density exceeds the dynamic binding capacity of the ion exchange chromatography material for the polypeptide; c) collecting fractions comprising the polypeptide; d) washing the anion exchange chromatography material with equilibration buffer; e) passing about 10 material volumes of a regeneration buffer through the anion exchange chromatography material, wherein the regeneration buffer comprises about 25 mM Tris and about 85 mM NaOAc at about pH 8.0; f) passing about 10 material volumes of sanitization buffer through the anion exchange chromatography material, wherein the sanitization buffer comprises about 0.5 N NaOH; and g) storing the anion exchange chromatography material in about 4 material volumes of a storage buffer, wherein the storage buffer comprises about 0.1 N NaOH. In some embodiments, the load density of the composition exceeds the dynamic binding capacity of the chromatography material for the polypeptide by between 2× and 100×. In some embodiments, the load density of the composition exceeds the dynamic binding capacity of the chromatography material for the polypeptide by about 10×.

In some embodiments of any of the methods described herein, the chromatography material is an ion exchange material (e.g., a cation exchange material or an anion exchange material). In some embodiments, the ion exchange material is in a chromatography column (e.g., a cation exchange chromatography column or an anion exchange chromatography column).

In some embodiments, the cation exchange material is a solid phase that is negatively charged and has free cations for exchange with cations in an aqueous solution passed over or through the solid phase. In some embodiments of any of the methods described herein, the cation exchange material may be a membrane, a monolith, or resin. In some embodiments, the cation exchange material may be a resin. The cation exchange material may comprise a carboxylic acid functional group or a sulfonic acid functional group such as, but not limited to, sulfonate, carboxylic, carboxymethyl sulfonic acid, sulfoisobutyl, sulfoethyl, carboxyl, sulphopropyl, sulphonyl, sulphoxyethyl, or orthophosphate. In some embodiments of the above, the cation exchange chromatography material is a cation exchange chromatography resin. In some embodiments of the above, the cation exchange chromatography material is a cation exchange chromatography membrane. In some embodiments of the invention, the chromatography material is not a cation exchange chromatography material. In some embodiments, the cation exchange chromatography material is used for large-scale purification of a polypeptide; e.g., in manufacturing-scale production of polypeptides such as antibodies or fragments thereof.

In some embodiments of any of the methods described herein, the ion exchange material may utilize a conventional chromatography material or a convective chromatography material. The conventional chromatography materials include, for example, perfusive materials (e.g., poly (styrene-divinylbenzene) resin) and diffusive materials (e.g., cross-linked agarose resin). In some embodiments, the poly (styrene-divinylbenzene) resin can be Poros™ resin. In some embodiments, the cross-linked agarose resin may be sulphopropyl-Sepharose® Fast Flow (‘SPSFF’) resin. The convective chromatography material may be a membrane (e.g., polyethersulfone) or monolith material (e.g. cross-linked polymer). The polyethersulfone membrane may be Mustang. The cross-linked polymer monolith material may be cross-linked poly(glycidyl methacrylate-co-ethylene dimethacrylate).

Examples of cation exchange materials are known in the art include, but are not limited to Mustang® S, Sartobind® S, SO3 Monolith, S Ceramic HyperD™, Poros™ XS, Poros™ HS50, Poros™ HS20, SPSFF, SP-Sepharose® XL (SPXL), CM Sepharose® Fast Flow, Capto™ S, Fractogel® Se HiCap, Fractogel® SO3, or Fractogel® COO. In some embodiments of any of the methods described herein, the cation exchange material is Poros™ HS50. In some embodiments, the Poros™ HS resin may be Poros™ HS 50 μm or Poros™ HS 20 μm particles.

In some aspects of the invention, the anion exchange chromatography material is a solid phase that is positively charged and has free anions for exchange with anions in an aqueous solution passed over or through the solid phase. In some embodiments of any of the methods described herein, the anion exchange material may be a membrane, a monolith, or resin. In an embodiment, the anion exchange material may be a resin. In some embodiments, the anion exchange material may comprise a primary amine, a secondary amine, a tertiary amine or a quaternary ammonium ion functional group, a polyamine functional group, or a diethylaminoethyl functional group. In some embodiments of the above, the anion exchange chromatography material is an anion exchange chromatography column. In some embodiments of the above, the anion exchange chromatography material is an anion exchange chromatography membrane. Examples of anion exchange materials are know in the art and include, but are not limited to Poros™ HQ 50, Poros™ PI 50, Poros™ D, Mustang® Q, Q Sepharose® FF, and DEAE Sepharose®.

In some embodiments, the ion exchange chromatography material is a mixed mode ion exchange chromatography material. Mixed mode chromatography materials comprise functional groups capable of one of more of the following functionalities: anionic exchange, cation exchange, hydrogen bonding, and hydrophobic interactions. In some embodiments, the ion exchange chromatography material is a mixed mode cation exchange chromatography material. Examples of cation exchange mixed mode material include but are not limited to Capto MMC™, Capto MMC™ ImpRes, Nuvia™ cPrime™, and Toyopearl MX Trp-650M. In some embodiments, the ion exchange chromatography material is a mixed mode anion exchange chromatography material. Examples of anion exchange mixed mode material include but are not limited to Capto Adhere™ and Capto Adhere™ ImpRes.

In some embodiments, the ion exchange chromatography material is used to purify multiple polypeptide products. In some embodiments, the ion exchange chromatography material is used to purify multiple antibody products. In some embodiments, the carryover after the cleaning methods comprises one or more of <0.25 mg/mL total protein, <1 ppm IgG fragments, <1 ppm IgG aggregates, <1 ppm leached Protein A, <1 μg/mL CZE LIF, <1 ppm CHOP, and <1 pg/mL CHO DNA.

In some embodiments, the ion exchange chromatography is combined with one or more other chromatography procedures in the purification of a polypeptide. In some embodiments, the ion exchange chromatography is combined with one or more other chromatography procedures in the purification of an antibody. In some embodiments, the ion exchange chromatography is combined with one or more other chromatography procedures in the large-scale purification of a polypeptide; e.g., in manufacturing-scale production of polypeptides such as antibodies or fragments thereof. In some embodiments, the ion exchange chromatography is combined with one or more other chromatography procedures in the large-scale purification of a polypeptide; e.g. in manufacturing-scale production of polypeptides such as antibodies or fragments thereof. In some embodiments, the one or more other chromatography procedures are performed prior to the overload ion exchange chromatography. In some embodiments, the one or more other chromatography procedures are performed after the overload ion exchange chromatography. In some embodiments, one or more other chromatography procedures are performed prior to the overload ion exchange chromatography and one or more other chromatography procedures are performed after the overload ion exchange chromatography. In some embodiments, the overload ion exchange chromatography is combined with one or more chromatography procedures selected from the group consisting of mixed mode chromatography, cation exchange chromatography, anion exchange chromatography, a hydrophobic interaction chromatography (HIC), affinity chromatography, and size-exclusion chromatography.

In some embodiments, the cation exchange chromatography is combined with one or more other chromatography procedures in the purification of a polypeptide. In some embodiments, the cation exchange chromatography is combined with one or more other chromatography procedures in the purification of an antibody. In some embodiments, the cation exchange chromatography is combined with one or more other chromatography procedures in the large-scale purification of a polypeptide; e.g., in manufacturing-scale production of polypeptides such as antibodies or fragments thereof. In some embodiments, the cation exchange chromatography is combined with one or more other chromatography procedures in the large-scale purification of a polypeptide; e.g., in manufacturing-scale production of polypeptides such as antibodies or fragments thereof. In some embodiments, the one or more other chromatography procedures are performed prior to the overload cation exchange chromatography. In some embodiments, the one or more other chromatography procedures are performed after the overload cation exchange chromatography. In some embodiments, one or more other chromatography procedures are performed prior to the overload cation exchange chromatography and one or more other chromatography procedures are performed after the overload cation exchange chromatography. In some embodiments, the overload cation exchange chromatography is combined with one or more chromatography procedures selected from the group consisting of mixed mode chromatography, anion exchange chromatography, a hydrophobic interaction chromatography (HIC), affinity chromatography, size-exclusion chromatography and additional cation exchange chromatography.

An example of a 2000 L purification process for a monoclonal antibody includes harvesting CHO cells producing the antibody to generate a feedstream. Subjecting the feedstream to a Protein A affinity chromatography (e.g., MabSelect SuRe™) in bind and elute mode at a load density≤30 g/L resin, subjecting the Protein A eluate to cation exchange chromatography (e.g. POROS™ XS) in bind and elute mode at a load density of ≤85 g/L resin, subjecting the cation exchange eluate to a mixed-mode anion exchange chromatography (e.g., Q-Sepharose® FF) in flow-through mode at a load density of ≤100 g/L resin, and subjecting the mixed-mode anion exchange fractions to ultrafiltration/diafiltration (e.g., Pellicon® 3) to formulate the antibody at a final concentration of 50 g/L.

Another example of a 2000 L purification process for a monoclonal antibody includes harvesting CHO cells producing the antibody to generate a feedstream. Subjecting the feedstream to a Protein A affinity chromatography (e.g., MabSelect SuRe™) in bind and elute mode at a load density≤30 g/L resin, subjecting the Protein A eluate to cation exchange chromatography (e.g. POROS™ HS) in bind and elute mode at a load density of ≤41 g/L resin, subjecting the cation exchange eluate to a mixed-mode anion exchange chromatography (e.g., Q-Sepharose® FF) in flow-through mode at a load density of ≤100 g/L resin, subjecting the mixed-mode anion exchange fractions a filter to remove virus (e.g., 1:1 Viresolve® ProShield/Viresolve® Pro) at 1.2-2.6 kg/m², and subjecting feedstream to ultrafiltration/diafiltration to formulate the antibody at a final concentration of 50 g/L.

An example of a 12000 L manufacturing-scale purification process for a monoclonal antibody includes harvesting CHO cells producing the antibody to generate a feedstream. Subjecting the feedstream to a detergent virus inactivation step (e.g., Triton-CG110), subjecting the feedstream to a Protein A affinity chromatography (e.g., MabSelect SuRe™) in bind and elute mode at a load density≤40 g/L resin, subjecting the Protein A eluate to cation exchange chromatography (e.g. POROS™ HS) in overload mode at a load density of ≤1000 g/L resin, subjecting the cation exchange eluate to a mixed-mode anion exchange chromatography (e.g., Capto™ Adhere) in flow-through mode at a load density of ≤350 g/L resin, and subjecting the mixed-mode anion exchange fractions to ultrafiltration/diafiltration (e.g., Pellicon® 3) to formulate the antibody at a final concentration of 60 g/L.

Another example of a 12000 L manufacturing-scale purification process for a monoclonal antibody includes harvesting CHO cells producing the antibody to generate a feedstream. Subjecting the feedstream to a Protein A affinity chromatography (e.g., MabSelect SuRe™) in bind and elute mode at a load density≤35 g/L resin, subjecting the Protein A eluate to cation exchange chromatography (e.g. POROS™ HS) in overload mode at a load density of ≤800 g/L resin, subjecting the cation exchange eluate to a mixed-mode anion exchange chromatography (e.g., Capto™ Adhere) in flow-through mode at a load density of ≤250 g/L resin, subjecting the mixed-mode anion exchange fractions a filter to remove virus (e.g., 1:1 Viresolve® ProShield/Viresolve® Pro) at 9-10 kg/m², and subjecting feedstream to ultrafiltration/diafiltration to formulate the antibody at a final concentration of 100 g/L.

In some aspects of the invention, the overload cation exchange chromatography is used in combination with an affinity chromatography material. Examples of affinity chromatography materials include, but are not limited to chromatography materials derivatized with Protein A and/or Protein G. Examples of affinity chromatography material include, but are not limited to, Prosep®-VA, Prosep®-VA Ultra Plus, Protein A Sepharose® fast flow, Toyopearl® Protein A. MAbSelect, MAbSelect SuRe™ and MAbSelect SuRe™ LX, protein L, FcXL, protein XL, kappa chromatography, and kappaXL chromatography. In some embodiments of the above, the affinity chromatography material is an affinity chromatography column. In some embodiments of the above, the affinity chromatography material is an affinity chromatography membrane.

In some aspects of the invention, the overload ion exchange chromatography (e.g., overload cation exchange chromatography) is used in combination with a mixed mode chromatography material. In some embodiments ofany of the methods described herein, the chromatography material is a mixed mode material comprising functional groups capable of one of more of the following functionalities: anionic exchange, cation exchange, hydrogen bonding, and hydrophobic interactions. In some embodiments, the mixed mode material comprises functional groups capable of anionic exchange and hydrophobic interactions. The mixed mode material may contain N-benzyl-N-methyl ethanol amine, 4-mercapto-ethyl-pyridine, hexylamine, or phenylpropylamine as ligand or contain cross-linked polyallylamine. Examples of the mixed mode materials include Capto™ Adhere resin, QMA resin, Capto™ MMC resin, MEP HyperCel™ resin, HEA HyperCel™ resin, PPA HyperCel™ resin, or ChromaSorb™ membrane or Sartobind® STIC. In some embodiments, the mixed mode material is Capto™ Adhere resin. In some embodiments of the above, the mixed mode material is a mixed mode chromatography column. In some embodiments of the above, the mixed mode material is a mixed mode membrane.

In some aspects of the invention, the overload ion exchange chromatography (e.g., overload cation exchange chromatography) is used in combination with an anion exchange chromatography material. In some embodiments, the anion exchange chromatography material is a solid phase that is positively charged and has free anions for exchange with anions in an aqueous solution passed over or through the solid phase. In some embodiments of any of the methods described herein, the anion exchange material may be a membrane, a monolith, or resin. In an embodiment, the anion exchange material may be a resin. In some embodiments, the anion exchange material may comprise a primary amine, a secondary amine, a tertiary amine or a quaternary ammonium ion functional group, a polyamine functional group, or a diethylaminoethyl functional group. In some embodiments of the above, the anion exchange chromatography material is an anion exchange chromatography column. In some embodiments of the above, the anion exchange chromatography material is an anion exchange chromatography membrane.

In some aspects of the invention, the overload ion exchange chromatography (e.g., overload cation exchange chromatography) is used in combination with a hydrophobic interaction chromatography material. Hydrophobic interaction chromatography (HIC) is a liquid chromatography technique that separates biomolecules according to hydrophobicity. Examples of HIC chromatography materials include, but are not limited to. Toyopearl® hexyl 650, Toyopcarl® butyl 650, Toyopcarl® phenyl 650, Toyopcarl® ether 650, Source, Resource, Sepharose® Hi-Trap, Octyl Sepharose®, Phenyl Sepharose®. In some embodiments of the above, the HIC chromatography material is a HIC chromatography column. In some embodiments of the above, the HIC chromatography material is a HIC chromatography membrane.

In some aspects of the invention, the overload ion exchange chromatography (e.g., overload cation exchange chromatography) is used in combination with a hydroxyapatite (HA) chromatography material. Examples of hydroxyapatite chromatography material include but am limited to HA Ultrogel®, and CHT hydroxyapatite. In some embodiments of the above, the HA chromatography material is a HA chromatography column. In some embodiments of the above, the HA chromatography material is a HA chromatography membrane.

In some embodiments, the overload ion exchange chromatography is performed in a column (i.e., column chromatography). In some embodiments, the one or more other chromatography procedures are performed in columns.

In some embodiments, the invention provides methods to clean or regenerate an alkali stable chromatography material; e.g. an alkali stable chromatography column.

In some embodiments of any of the methods described herein, the flow rate is less than about any of 50 material volumes/hr, 40 material volumes/hr, or 30 material volumes/hr. The flow rate may be between about any of 5 material volumes/hr and 50 material volumes/hr, 10 material volumes/hr and 40 material volumes/hr. or 18 material volumes/hr and 36 material volumes/hr. In some embodiments, the flow rate is about any of 9 material volumes/hr, 18 material volumes/hr, 25 material volumes/hr, 30 material volumes/hr, 36 material volumes/hr, or 40 material volumes/hr.

In some embodiments, the chromatography material is in a chromatography column. In some embodiments of any of the methods described herein, the flow rate is less than about any of 50 column volumes (CV)/hr, 40 CV/hr, 30 CV/hr, 20 CV/hr, or 15 CV/hr. The flow rate may be between about any of 5 CV/hr and 50 CV/hr, 10 CV/hr and 40 CV/hr. or 15 CV/hr and 20 CV/hr. In some embodiments, the flow rate is about any of 10 CV/hr, 15 CV/hr, 20 CV/hr, 25 CV/hr, 30 CV/hr, or 40 CV/hr. In some embodiments of any of the methods described herein, the flow rate is less than about any of 100 cm/hr, 75 cm/hr, or 50 cm/hr. The flow rate may be between about any of 25 cm/hr and 150 cm/hr, 25 cm/hr and 100 cm/hr, 50 cm/hr and 100 cm/hr, or 65 cm/hr and 85 cm/hr.

Bed height is the height of chromatography material used. In some embodiments of any of the method described herein, the bed height is greater than about any of 3 cm, 10 cm, or 15 cm. The bed height may be between about any of 3 cm and 35 cm, 5 cm and 15 cm, 3 cm and 10 cm, or 5 cm and 8 cm. In some embodiments, the bed height is about any of 3 cm, 5 cm, 10 cm, 15 cm, 20 cm, 25 cm, or 30 cm. In some embodiments, bed height is determined based on the amount of polypeptide or contaminants in the load. In some embodiments, the chromatography material is in a column used for large-scale purification of a polypeptide; e.g., in manufacturing-scale production of polypeptides such as antibodies or fragments thereof. In some embodiments, the manufacturing-scale chromatography material has a bed height of about any of 10 cm, 15, cm, 20 cm, 25 cm or 30 cm.

Bed diameter is the diameter of chromatography material used. In some embodiments of any of the method described herein, the bed diameter is greater than about any of 80 cm, 100 cm, or 120 cm. In some embodiments, the bed diameter is about any of 50 cm, 60 cm, 70 cm, 80 cm, 90 cm, 100 cm, 110 cm, 120 cm, 130 cm, 140 cm, 150 cm, 160 cm, 170 cm, 180 cm, 190, or 200 cm. In some embodiments, bed diameter is determined based on the amount of polypeptide or contaminants in the load. In some embodiments, the chromatography material is in a column used for large-scale purification of a polypeptide; e.g., in manufacturing-scale production of polypeptides such as antibodies or fragments thereof. In some embodiments, the manufacturing-scale chromatography material had a bed diameter of about any 50 cm, 60 cm, 70 cm, 80 cm, 90 cm, 100 cm, 110 cm, 120 cm, 130 cm, 140 cm, 150 cm, 160 cm, 170 cm, 180 cm, 190, or 200 cm.

In some embodiments, the chromatography is in a column or vessel with a volume of greater than about 1 mL, 2 mL, 3 mL, 4 mL, 5 mL, 6 mL, 7 mL, 8 mL, 9 mL, 10 mL, 15 mL, 20 mL, 25 mL, 30 mL, 40 mL, 50 mL, 75 mL, 100 mL, 200 mL, 300 mL, 400 mL, 500 mL, 600 mL, 700 mL, 800 mL, 900 mL, 1 L, 2 L, 3 L, 4 L, 5 L, 6 L, 7 L, 8 L, 9 L, 10 L, 25 L, 50 L, 100 L, 200 L, 300 L, 400 L, 500 L, 600 L, 700 L, 800 L, 900 L or 1000 L. In some embodiments, the vessel has a bed height of 14 cm and a bed volume of 80 cm; e.g. a large-scale Protein A column. In some embodiments, the vessel has a bed height of 19 cm and a bed volume of 100 cm. e.g. a large-scale anion exchange column. In some embodiments, the vessel has a bed height of 30 cm and a bed volume of 120 cm. e.g. a large-scale ion exchange column.

Load, as used herein, is the composition loaded onto a chromatography material. In some embodiments, the load is a polypeptide that is loaded onto a chromatography material that had been previously used to isolate a different polypeptide. Loading buffer is the buffer used to load the composition comprising the product of interest onto a chromatography material. The chromatography material may be equilibrated with an equilibration buffer prior to loading the composition which is to be purified. In some examples, a wash buffer is used after loading the composition onto a chromatography material and before elution of the polypeptide of interest from the solid phase. In some embodiments, the wash buffer is the same as the load buffer. In some embodiments, some of the product of interest, e.g. a polypeptide, may be removed from the chromatography material by the wash buffer (e.g. overload mode).

Elution, as used herein, is the removal of the product, e.g. polypeptide, from the chromatography material. In some embodiments of the invention, the elution is a “mock elution” where an elution procedure is applied to a chromatography material for which a protein was not loaded subsequent of the last cleaning procedure. In some embodiments of the invention, the mock elution procedure is applied to a chromatography material following any one of the cleaning procedure described herein. In some embodiments, the mock elution mimics the elution that will be used to elute a protein that will be applied to the material in an effort to determine if there may be carryover material (e.g., contaminants) during the actual production run. A mock elution can be used as a means to evaluate the efficacy of the cleaning procedure.

Elution buffer is the buffer used to elute the polypeptide or other product of interest from a chromatography material. In many cases, an elution buffer has a different physical characteristic than the load buffer. For example, the elution buffer may have a different conductivity than load buffer or a different pH than the load buffer. In some embodiments, the elution buffer has a lower conductivity than the load buffer. In some embodiments, the elution buffer has a higher conductivity than the load buffer. In some embodiments, the elution buffer has a lower pH than the load buffer. In some embodiments, the elution buffer has a higher pH than the load buffer. In some embodiments, the elution buffer has a different conductivity and a different pH than the load buffer. The elution buffer can have any combination of higher or lower conductivity and higher or lower pH.

Conductivity refers to the ability of an aqueous solution to conduct an electric current between two electrodes. In solution, the current flows by ion transport. Therefore, with an increasing amount of ions present in the aqueous solution, the solution will have a higher conductivity. The basic unit of measure for conductivity is the Siemen (or mho), mho (mS/cm), and can be measured using a conductivity meter, such as various models of Orion conductivity meters. Since electrolytic conductivity is the capacity of ions in a solution to carry electrical current, the conductivity of a solution may be altered by changing the concentration of ions therein. For example, the concentration of a buffering agent and/or the concentration of a salt (e.g. sodium chloride, sodium acetate, or potassium chloride) in the solution may be altered in order to achieve the desired conductivity Preferably, the salt concentration of the various buffers is modified to achieve the desired conductivity.

### Contaminants

The invention provides methods for the reuse of chromatography materials for use on a large-scale purification of a polypeptide; e.g., in manufacturing-scale production of polypeptides such as antibodies or fragments thereof. The method provides for the multiple uses of chromatography materials for multiple polypeptide products. For example, using the methods of the invention, a first antibody can be purified at an industrial scale on a chromatography material, followed by the methods of cleaning/regenerating the chromatography material described herein, and then followed by the industrial scale purification of a second antibody product. In some embodiments, the methods of the invention are used to reduce the “carryover” of previous products that were purified using the chromatography material. In some embodiments, the carryover contaminants include but are not limited to whole antibodies, IgG fragments, Fc, Fc fragments, and antibody aggregates.

In some embodiments of any of the methods described herein, the at least one contaminant is any one or more of host cell material, such as CHOP; leached Protein A; nucleic acid; a variant, fragment, aggregate or derivative of the desired polypeptide; another polypeptide; endotoxin; viral contaminant; cell culture media component, carboxypeptidase B, gentamicin, etc. In some examples, the contaminant may be a host cell protein (HCP) from, for example but not limited to, a bacterial cell such as an E. coli cell, an insect cell, a prokaryotic cell, a eukaryotic cell, a yeast cell, a mammalian cell, an avian cell, a fungal cell.

Leached Protein A is Protein A detached or washed from a solid phase to which it is bound. For example, leached Protein A can be leached from Protein A chromatography material. The amount of Protein A may be measured, for example, by ELISA. In some embodiments of any of the methods described herein, the amount of leached Protein A is reduced by greater than about any of 10%, 20%, 30%, 40%, 50%, 60%, 70%, 80%, or 90%. The amount of leached Protein A may be reduced by between about any of 10% and 99%, 30% and 95%, 30% and 99%, 50% and 95%, 50% and 99%, 75% and 99%, or 85% and 99%. In some embodiments, the amount of leached Protein A is reduced by about any of 10%, 20%, 30%, 40%, 50%, 60%, 70%, 80%, 90%, or 95%. In some embodiments, the reduction is determined by comparing the amount of leached Protein A in the composition recovered from a purification step(s) to the amount of leached Protein A in the composition before the purification step(s).

Host cell proteins (HCP) are proteins from the cells in which the polypeptide was produced. For example, CHOP are proteins from host cells, i.e., Chinese Hamster Ovary Proteins. The amount of CHOP may be measured by enzyme-linked immunosorbent assay (“ELISA”) or Meso Scale Discovery (“MSO”). In some embodiments of any of the methods described herein, the amount of HCP (e.g. CHOP) in the eluate is at a minimum in a mock elution. In some embodiments, the level of host cell protein in an eluate from a mock elution is compared with and without cleaning method or before and after cleaning method.

Methods of measuring DNA such as host cell DNA are known in the art and described in the examples section. In some embodiments of any of the methods described herein, the amount of DNA is reduced by greater than about any of 10%, 20%, 30%, 40%, 50%, 60%, 70%, 80%, or 90%. The amount of DNA may be reduced by between about any of 10% and 99%, 30% and 95%, 30% and 99%, 50% and 95%, 50% and 99%, 75% and 99%, or 85% and 99%. The amount of DNA may be reduced by about any of 10%, 20%, 30%, 40%, 50%, 60%, 70%, 80%, 90%, 95%, or 99%. In some embodiments, the reduction is determined by comparing the amount of DNA in the composition recovered from a purification step(s) to the amount of DNA in the composition before the purification step(s).

Fragment polypeptide can be low molecular weight (LMW) protein. In some embodiments, the fragmented polypeptide is a fragment of the polypeptide of interest. Examples of LMW protein include, but not limited to, a Fab (Fragment antigen binding), Fc (fragment, crystallizable) regions or combination of both or any random fragmented part of an antibody of interest. Methods of measuring fragmented protein (e.g., LMW protein) are known in the art and described in the examples section. In some embodiments of any of the methods described herein, the amount of LMW protein is reduced by greater than about any of 5%, 10%, 20%, 30%, 40%, 50%, 60%, 700, 80%, 90%, or 95%. The amount of LMW protein may be reduced by between about any of 10% and 99%, 30% and 95%, 30% and 99%, 50% and 95%, 50% and 99%, 75% and 99%, or 85% and 99%. The amount of LMW protein may be reduced by about any of 5%, 10%, 20%, 30%, 40%, 50%, 60%, 70%, 80%, 90%, or 95%. In some embodiments, the reduction is determined by comparing the amount of fragmented protein (e.g., LMW protein) in the composition recovered from a purification step(s) to the amount of fragmented protein (e.g., LMW protein) in the composition before the purification step(s).

Aggregated polypeptide can be high molecular weight (HMW) protein. In some embodiments, the aggregated polypeptide is multimers of the polypeptide of interest. The HMW protein may be a dimer, up to 8× monomer, or larger of the polypeptide of interest. Methods of measuring aggregated protein (e.g., HMW protein) are known in the art. In some embodiments, the level of HMW in a mock elution is at a minimum; e.g., less than about 5 ppm, less than about 4 ppm, less than about 3 ppm, less than about 2 ppm or less than about 1 ppm. In some embodiments of any of the methods described herein, the amount of aggregated protein is reduced by greater than about any of 5%, 10%, 20%, 30%, 40%, 50%, 60%, 70%, 80%, 90%, or 95%. The amount of aggregated protein may be reduced by between about any of 10% and 99%, 30% and 95%, 30% and 99%, 50% and 95%, 50% and 99%, 75% and 99%, or 85% and 99%. The amount of aggregated protein may be reduced by about any of 5%, 10%, 20%, 30%, 40%, 50%, 60%, 70%, 80%, 90%, or 95%. In some embodiments, the reduction is determined by comparing the amount of aggregated protein (e.g., HMW protein) in the composition recovered from a purification step(s) to the amount of aggregated protein (e.g., HMW protein) in the composition before the purification step(s).

Cell culture media component refers to a component present in a cell culture media. A cell culture media may be a cell culture media at the time of harvesting cells. In some embodiments, the cell culture media component is gentamicin. The amount of gentamicin may be measured by ELISA. In some embodiments of any of the methods described herein, the amount of cell culture media component is reduced by greater than about any of 10%, 20%, 30%, 40%, 50%, 60%, 70%, 80%, or 90%. The amount of cell culture media component may be reduced by between about any of 10% and 99%, 30% and 95%, 30% and 99%, 50% and 95%, 50% and 99%, 75% and 99%, or 85% and 99%. In some embodiments, the amount of cell culture media component is reduced by about any of 10%, 20%, 30%, 40%, 50%, 60%, 70%, 80%, 90%, 95%, or 98%. In some embodiments, the reduction is determined by comparing the amount of cell culture media component in the composition recovered from a purification step(s) to the amount of cell culture media component in the composition before the purification step(s).

### Methods to Detect Contaminants

The invention provides methods to evaluate the effectiveness of the cleaning of the reusable chromatography material. For example, a chromatography material in which a polypeptide had been previously loaded and eluted at least one time is cleaned by one of the methods of the invention described above. A mock elution is then run on the material where no additional polypeptide had been loaded on the material after the cleaning procedure. The mock elution may follow the elution procedure that was used on the polypeptide that was previously loaded on the material or the elution procedure may follow the elution procedure for the polypeptide that will be purified after the cleaning procedure. In some embodiments, a mock loading is run on the material prior to the mock elution. A mock loading uses the same procedure to load a polypeptide on the material with the exception that the polypeptide is not included in the loading. In some embodiments, the eluent from the mock elution is collected in one or more fractions. In some embodiments, the eluent of the mock elution is collected in a single fraction. In some embodiments, the eluent, or a sample of the eluent, is analyzed for contaminants including carryover polypeptides from the previous loading of the chromatography material, IgG fragments, leached Protein A, CHOP and CHO DNA.

### Polypeptide Quantification

The concentration of polypeptide such as a monoclonal antibody (mAb) can be determined via absorbance at 280 and 320 nm using a UV-visible spectrophotometer (8453 model G1103A; Agilent Technologies; Santa Clara, Calif., U.S.A.) or NanoDrop™ 1000 model ND-1000 (Thermo Fisher Scientific; Waltham, Mass., U.S.A.). Species other than the polypeptide previously loaded on the reusable chromatography material or the polypeptide loaded onto a material cleaned by the methods of the invention (i.e. impurities) may be too low in concentration to have an appreciable effect on UV absorbance. As needed, samples may be diluted with an appropriate non-interfering diluent in the range of 0.1-1.0 absorbance unit. Sample preparation and UV measurement are performed in duplicate and the average value is recorded. MAb absorption coefficients may range from 1.42 to 1.645/mg·ml·cm.

Total protein can be determined by a capillary zone electrophoresis/Laser-induced fluorescence detection assay.

### IgG Detection

Intact human IgG and human IgG fragments may be detected using an intact human IgG-specific or IgG fragment-specific ELISA. Human Fc may be detected using a human Fc-specific ELISA.

### IgG Aggregates

IgG aggregates may be detected using size exclusion chromatography. IgG aggregates are typically larger than intact IgGs.

### CHO Host Cell Protein (CHOP) Quantification

An ELISA may be used to quantify the levels of the CHO host cell protein called CHOP. Anti-CHOP antibodies are immobilized on microtiter plate wells Dilutions of the samples containing CHOP, standards and controls are incubated in the wells, followed by incubation with anti-CHOP antibodies conjugated with horseradish peroxidase (HRP). The HRP enzymatic activity can be detected with o-phenylenediamine, and the CHOP is quantified by reading absorbance at 490 nm in a microtiter plate reader. Based on the principles of sandwich ELISA, the concentration of peroxidase corresponds to the CHOP concentration. The assay range for the ELISA is typically 5-320 ng/ml with intra-assay variability <10%. CHOP values may be reported in units of ng/ml. Alternatively, CHOP values may be divided by the polypeptide concentration and the results may be reported in PPM (parts per million; e.g. ng of CHOP/mg of polypeptide). The CHOP ELISA may be used to quantify total CHOP levels in a sample but docs not quantify the concentration of individual proteins.

### CHO DNA Quantification

CHO DNA in product samples may be quantified using real-time PCR (TaqMan PCR). DNA from samples and controls may first be extracted using Qiagen's Virus Biorobot kit. The extracted samples, controls, and standard DNA, are subject to TaqMan real time Polymerase chain reaction (PCR) using PCR primers and probe in a 96-well plate with ABI's sequence detection system. The primers are defined by a 110 base pair segment of a repetitive DNA sequence in the Cricetulus griseus genome. The probe is labeled with a fluorescent reporter dye at 5′ end and a quencher dye at the 3′ end. When the probe is intact, the emission spectrum of the reporter is suppressed by the quencher. The 5′ nuclease activity of polymerase hydrolyzes the probe and releases the report, which results in an increase in fluorescence emission. The sequence detector quantifies the amplified product in direct proportion to the increase in fluorescence emission measured continuously during the DNA amplification. Cycle numbers at which DNA had amplifies past the threshold (CT) are calculated for the standard curve. A standard curve ranging 1 pg/mL-10,000 pg/mL may be generated, which is used for quantifying DNA in samples.

### Leached Protein A Quantification

The level of leached Protein-A in the Protein A pools may be determined by a sandwich Protein-A ELISA. Chicken anti-staphylococcal Protein A antibodies are immobilized on microtiter plate wells. The sample treatment procedure may include sample dilution and dissociation of the Protein A/IgG complex using microwave assisted heating as a pretreatment step before running the samples on a sandwich ELISA. Protein A, if present in the sample, may bind to the coated antibody. Bound Protein A is detected using horseradish peroxidase conjugated anti-protein antibodies. Horseradish peroxidase enzymatic activity is quantified with a two component TMB substrate solution which produces a colorimetric signal.

### Polypeptides

The methods of the invention may be used to clean chromatography material used in the purification of multiple polypeptides. In some embodiments, the chromatography material is used in large-scale; e.g., in manufacturing-scale production of polypeptides such as antibodies or fragments thereof. In some embodiments, a chromatography material is used in the purification of a first polypeptide, such as a first antibody, the material is then cleaned by the methods of the invention, and then the chromatography material can be used to purify a second polypeptide, such as a second antibody. In some embodiments, the cleaning is effective such that the preparation comprising the second purified polypeptide is essentially free of the first polypeptide. In some embodiments, the preparation comprising the second purified polypeptide (e.g. a second antibody) comprises less than 1 ppm of the first polypeptide (e.g. a first antibody) In some embodiments, the second purified polypeptide comprises less than any one of 1 ppm, 2 ppm, 3 ppm, 4 ppm, 5 ppm, 10 ppm, 20 ppm, 30 ppm, 40 ppm, 50 ppm or 100 ppm of the first polypeptide.

In some embodiments, the methods of the invention are used to reuse chromatography material used to purify therapeutic polypeptides. In some embodiments, the polypeptide is an antagonist. In some embodiments, the polypeptide is an agonist. In some embodiments, the polypeptide is an antibody. In some embodiments, the polypeptide is epitope tagged. In some embodiments, the polypeptide retains a biological and/or immunological activity. In some embodiments, the polypeptide is an antagonist. In some embodiments, the polypeptide initiates complement dependent cytotoxicity. In some embodiments the polypeptide is an antibody or immunoadhesin.

In some embodiments, the polypeptide, the first polypeptide and/or the second polypeptide, has a molecular weight of greater than about any of 5,000 Daltons, 10,000 Daltons, 15,000 Daltons, 25,000 Daltons, 50,000 Daltons, 75,000 Daltons, 100,000 Dalton, 125,000 Daltons, or 150,000 Daltons. The polypeptide may have a molecular weight between about any of 50,000 Daltons to 200,000 Daltons or 100,000 Daltons to 200,000 Daltons. Alternatively, the polypeptide for use herein may have a molecular weight of about 120,000 Daltons or about 25,000 Daltons.

pI is the isoelectric point and is the pH at which a particular molecule or the surface of the molecule carries no net electrical charge. In some embodiments of any of the methods described herein, the pI of the polypeptide, e.g. the first polypeptide and/or the second polypeptide, may be between about any of 6 to 10, 7 to 9, or 8 to 9 In some embodiments, the polypeptide has a pi of about any of 6, 7, 7.5, 8, 8.5, 9, 9.5, or 10.

The polypeptides to be purified using reusable chromatography material cleaned by the methods described herein are generally produced using recombinant techniques Methods for producing recombinant proteins are described, e.g., in U.S. Pat. Nos. 5,534,615 and 4,816,567, specifically incorporated herein by reference. In some embodiments, the protein of interest is produced in a CHO cell (see, e.g. WO 94/11026). When using recombinant techniques, the polypeptides can be produced intracellularly, in the periplasmic space, or directly secreted into the medium.

The polypeptides to be purified using reusable chromatography material cleaned by the methods described herein may be recovered from culture medium or from host cell lysates. Cells employed in expression of the polypeptides can be disrupted by various physical or chemical means, such as freeze-thaw cycling, sonication, mechanical disruption, or cell lysing agents. If the polypeptide is produced intracellularly, as a first step, the particulate debris, either host cells or lysed fragments, are removed, for example, by centrifugation or ultrafiltration. Carter et al., Bio/Technology 10: 163-167 (1992) describe a procedure for isolating polypeptides which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the polypeptide is secreted into the medium, supernatants from such expression systems are generally first concentrated using a commercially available polypeptide concentration filter, for example, an Amicon® or Millipore Pellicon®, ultrafiltration unit. A protease inhibitor such as PMSF may be included in any of the foregoing steps to inhibit proteolysis and antibiotics may be included to prevent the growth of adventitious contaminants.

Examples of polypeptides that may be purified using reusable chromatography material cleaned by the methods described herein include but are not limited to immunoglobulins, immunoadhesins, antibodies, enzymes, hormones, fusion proteins, Fc-containing proteins, immunoconjugates, cytokines and interleukins. Examples of polypeptide include, but are not limited to, mammalian proteins, such as, e.g., renin; a hormone, a growth hormone, including human growth hormone and bovine growth hormone; growth hormone releasing factor; parathyroid hormone; thyroid stimulating hormone; lipoproteins; alpha-1-antitrypsin; insulin A-chain; insulin B-chain; proinsulin; follicle stimulating hormone; calcitonin; luteinizing hormone; glucagon; clotting factors such as factor VIIIC, factor IX, tissue factor, and von Willebrands factor, anti-clotting factors such as Protein C; atrial natriuretic factor, lung surfactant; a plasminogen activator, such as urokinase or human urine or tissue-type plasminogen activator (t-PA); bombesin; thrombin; hemopoietic growth factor; tumor necrosis factor-alpha and -beta; enkephalinase; RANTES (regulated on activation normally T-cell expressed and secreted); human macrophage inflammatory protein (MIP-1-alpha); a serum albumin such as human serum albumin; Muellerian-inhibiting substance; relaxin A-chain; relaxin B-chain; prorelaxin; mouse gonadotropin-associated peptide, an enzyme, a microbial protein, such as beta-lactamase; DNase; IgE; a cytotoxic T-lymphocyte associated antigen (CTLA), such as CTLA-4; inhibin; activin; vascular endothelial growth factor (VEGF); receptors for hormones or growth factors; Protein A or D; rheumatoid factors; a neurotrophic factor such as bone-derived neurotrophic factor (BDNF), neurotrophin-3, -4, -5, or -6 (NT-3, NT-4, NT-5, or NT-6), or a nerve growth factor such as NGF-b; platelet-derived growth factor (PDGF); fibroblast growth factor such as aFGF and bFGF; epidermal growth factor (EGF); transforming growth factor (TGF) such as TGF-alpha and TGF-beta, including TGF-β1, TGF-β2, TGF-β3, TGF-β4, or TGF-β5, insulin-like growth factor-I and -II (IGF-I and IGF-II); des(1-3)-IGF-I (brain IGF-I), insulin-like growth factor binding proteins (IGFBPs); a cytokine; CD proteins such as CD3, CD4, CD8, CD19 and CD20; erythropoietin; osteoinductive factors; immunotoxins; a fusion polypeptide. i.e. a polypeptide comprised on two or more heterologous polypeptides or fragments thereof and encoded by a recombinant nucleic acid; an Fc-containing polypeptide, for example, a fusion protein comprising an immunoglobulin Fc region, or fragment thereof, fused to a second polypeptide; an immunoconjugate; a bone morphogenetic protein (BMP); an interferon such as interferon-alpha, -beta, and -gamma; colony stimulating factors (CSFs), e.g., M-CSF. GM-CSF, and G-CSF; interleukins (ILs), e.g., IL-1 to IL-10; superoxide dismutase; T-cell receptors; surface membrane proteins; decay accelerating factor; viral antigen such as, for example, a portion of the AIDS envelope; transport proteins; homing receptors; addressins; regulatory proteins; integrins such as CD11a, CD11b, CD11c, CD18, an ICAM, VLA-4 and VCAM; a tumor associated antigen such as CA125 (ovarian cancer antigen) or HER2, HER3 or HER4 receptor; immunoadhesins; and fragments and/or variants of any of the above-listed proteins as well as antibodies, including antibody fragments, binding to a protein, including, for example, any of the above-listed proteins.

### Antibodies

In some embodiments of any of the methods described herein, the polypeptide that may be purified using reusable chromatography material cleaned by the methods described herein, e.g. the first polypeptide, the second polypeptide or any subsequent polypeptides, is an antibody.

Molecular targets for antibodies include CD proteins and their ligands, such as, but not limited to: (i) CD3, CD4, CD8, CD19, CD11a, CD20, CD22, CD34, CD40, CD79a (CD79a), and CD79β (CD79b); (ii) members of the ErbB receptor family such as the EGF receptor, HER2, HER3 or HER4 receptor; (iii) cell adhesion molecules such as LFA-1, Mac1, p150,95, VLA-4, ICAM-1, VCAM and αv/β3 integrin, including either alpha or beta subunits thereof (e.g., anti-CD11a, anti-CD18 or anti-CD11b antibodies); (iv) growth factors such as VEGF; IgE; blood group antigens; flk2/flt3 receptor; obesity (OB) receptor; mpl receptor; CTLA-4; protein C, BR3, c-met, tissue factor, β7, etc.; and (v) cell surface and transmembrane tumor-associated antigens (TAA), such as those described in U.S. Pat. No. 7,521,541.

Other exemplary antibodies include those selected from, and without limitation, anti-CD20 antibody, anti-CD40 antibody, anti-HER2 antibody, anti-IL6 antibody, anti-IgE antibody, anti-IL13 antibody, anti-Flu A antibody, anti-TIGIT antibody, anti-PD-L1 antibody, anti-VEGF-A antibody, anti-VEGF-A/ANG2 antibody, anti-CD79b antibody, anti-ST2 antibody, anti-factor D antibody, anti-factor IX antibody, anti-factor X antibody, anti-abeta antibody, anti-tau antibody, anti-CEA antibody, anti-CEA/CD3 antibody, anti-CD20/CD3 antibody, anti-FcRH5/CD3 antibody, anti-Her2/CD3 antibody, anti-FGFR1/KLB antibody, a FAP-4-1 BBL fusion protein, a FAP-IL2v fusion protein, anti-estrogen receptor antibody, anti-progesterone receptor antibody, anti-p53 antibody, anti-EGFR antibody, anti-cathepsin D antibody, anti-Bcl-2 antibody, anti-E-cadherin antibody, anti-CA125 antibody, anti-CA15-3 antibody, anti-CA19-9 antibody, anti-c-erbB-2 antibody, anti-P-glycoprotein antibody, anti-CEA antibody, anti-retinoblastoma protein antibody, anti-ras oncoprotein antibody, anti-Lewis X antibody, anti-Ki-67 antibody, anti-PCNA antibody, anti-CD3 antibody, anti-CD4 antibody, anti-CD5 antibody, anti-CD7 antibody, anti-CD8 antibody, anti-CD9/p24 antibody, anti-CD10 antibody, anti-CD11a antibody, anti-CD11c antibody, anti-CD13 antibody, anti-CD14 antibody, anti-CD15 antibody, anti-CD19 antibody, anti-CD22 antibody, anti-CD23 antibody, anti-CD30 antibody, anti-CD31 antibody, anti-CD33 antibody, anti-CD34 antibody, anti-CD35 antibody, anti-CD38 antibody, anti-CD41 antibody, anti-LCA/CD45 antibody, anti-CD45RO antibody, anti-CD45RA antibody, anti-CD39 antibody, anti-CD100 antibody, anti-CD95/Fas antibody, anti-CD99 antibody, anti-CD106 antibody, anti-ubiquitin antibody, anti-CD71 antibody, anti-c-myc antibody, anti-cytokeratins antibody, anti-vimentins antibody, anti-HPV proteins antibody, anti-kappa light chains antibody, anti-lambda light chains antibody, anti-melanosomes antibody, anti-prostate specific antigen antibody, anti-S-100 antibody, anti-tau antigen antibody, anti-fibrin antibody, anti-keratins antibody and anti-Tn-antigen antibody. In some embodiments, the antibody is selected from the group consisting of ocrelizumab, pertuzumab, trastuzumab, tocilizumab, faricimab, polatuzumab, gantenerumab, cibisatamab, crenezumab, mosunetuzumab, tiragolumab, bevacizumab, rituximab, atezolizumab, obinutuzumab, lampalizumab, lebrikizumab, omalizumab ranibizumab, emicizumab, selicrelumab, prasinezumab, RO6874281 and RO7122290.

### Polyclonal Antibodies

In some embodiments, the antibodies are polyclonal antibodies. Polyclonal antibodies are preferably raised in animals by multiple subcutaneous (sc) or intraperitoneal (ip) injections of the relevant antigen and an adjuvant. It may be useful to conjugate the relevant antigen to a polypeptide that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCl₂, or R¹N═C═NR, where R and R¹ are different alkyl groups.

Animals are immunized against the antigen, immunogenic conjugates, or derivatives by combining, e.g., 100 μg or 5 μg of the polypeptide or conjugate (for rabbits or mice, respectively) with 3 volumes of Freund's complete adjuvant and injecting the solution intradermally at multiple sites. One month later the animals are boosted with ⅕ to 1/10 the original amount of peptide or conjugate in Freund's complete adjuvant by subcutaneous injection at multiple sites. Seven to 14 days later the animals are bled and the serum is assayed for antibody titer. Animals are boosted until the titer plateaus. In some embodiments, the animal is boosted with the conjugate of the same antigen, but conjugated to a different polypeptide and/or through a different cross-linking reagent. Conjugates also can be made in recombinant cell culture as polypeptide fusions. Also, aggregating agents such as alum are suitably used to enhance the immune response.

### Monoclonal Antibodies

In some embodiments, the antibodies purified on reusable chromatography material cleaned by the methods of the invention are monoclonal antibodies. Monoclonal antibodies are obtained from a population of substantially homogeneous antibodies, i.e., the individual antibodies comprising the population are identical and/or bind the same epitope except for possible variants that arise during production of the monoclonal antibody, such variants generally being present in minor amounts. Thus, the modifier “monoclonal” indicates the character of the antibody as not being a mixture of discrete or polyclonal antibodies.

For example, the monoclonal antibodies may be made using the hybridoma method first described by Kohler et al. Nature 256:495 (1975), or may be made by recombinant DNA methods (U.S. Pat. No. 4,816,567).

In the hybridoma method, a mouse or other appropriate host animal, such as a hamster, is immunized as herein described to elicit lymphocytes that produce or are capable of producing antibodies that will specifically bind to the polypeptide used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies Principles and Practice, pp. 59-103 (Academic Press, 1986)).

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

In some embodiments, the myeloma cells are those that fuse efficiently, support stable high-level production of antibody by the selected antibody-producing cells, and are sensitive to a medium such as HAT medium. Among these, in some embodiments, the myeloma cell lines are murine myeloma lines, such as those derived from MOPC-21 and MPC-11 mouse tumors available from the Salk Institute Cell Distribution Center, San Diego, Calif. USA, and SP-2 or X63-Ag8-653 cells available from the American Type Culture Collection, Rockville, Md. USA. Human myeloma and mouse-human heteromyeloma cell lines also have been described for the production of human monoclonal antibodies (Kozbor, J. Immunol. 133:3001 (1984); Brodeur et al., Monoclonal Antibody Production Techniques and Applications pp. 51-63 (Marcel Dekker, Inc., New York, 1987)).

Culture medium in which hybridoma cells are growing is assayed for production of monoclonal antibodies directed against the antigen. In some embodiments, the binding specificity of monoclonal antibodies produced by hybridoma cells is determined by immunoprecipitation or by an in vitro binding assay, such as radioimmunoassay (RIA) or enzyme-linked immunoabsorbent assay (ELISA).

The binding affinity of the monoclonal antibody can, for example, be determined by the Scatchard analysis of Munson et al., Anal. Biochem. 107:220 (1980).

After hybridoma cells are identified that produce antibodies of the desired specificity, affinity, and/or activity, the clones may be subcloned by limiting dilution procedures and grown by standard methods (Goding, Monoclonal Antibodies: Principles and Practice pp. 59-103 (Academic Press, 1986)). Suitable culture media for this purpose include, for example, D-MEM or RPMI-1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, polypeptide A-Sepharose, hydroxylapatite chromatography, gel electrophoresis, dialysis, or affinity chromatography.

DNA encoding the monoclonal antibodies is readily isolated and sequenced using conventional procedures (e.g., by using oligonucleotide probes that are capable of binding specifically to genes encoding the heavy and light chains of murine antibodies). In some embodiments, the hybridoma cells serve as a source of such DNA. Once isolated, the DNA may be placed into expression vectors, which are then transfected into host cells such as E. coli cells, simian COS cells, Chinese Hamster Ovary (CHO) cells, or myeloma cells that do not otherwise produce immunoglobulin polypeptide, to obtain the synthesis of monoclonal antibodies in the recombinant host cells. Review articles on recombinant expression in bacteria of DNA encoding the antibody include Skerra et al., Curr. Opinion in Immunol. 5:256-262 (1993) and Plückthun, Immunol. Rev., 130:151-188 (1992).

In a further embodiment, antibodies or antibody fragments can be isolated from antibody phage libraries generated using the techniques described in McCafferty et al., Nature 348:552-554 (1990). Clackson et al., Nature 352:624-628 (1991) and Marks et al., J. Mol. Biol. 222:581-597 (1991) describe the isolation of murine and human antibodies, respectively, using phage libraries. Subsequent publications describe the production of high affinity (nM range) human antibodies by chain shuffling (Marks et al., Bio/Technology 10:779-783 (1992)), as well as combinatorial infection and in vivo recombination as a strategy for constructing very large phage libraries (Waterhouse et al., Nuc. Acids. Res. 21:2265-2266 (1993)). Thus, these techniques are viable alternatives to traditional monoclonal antibody hybridoma techniques for isolation of monoclonal antibodies.

The DNA also may be modified, for example, by substituting the coding sequence for human heavy- and light chain constant domains in place of the homologous murine sequences (U.S. Pat. No. 4,816,567; Morrison et al., Proc. Natl Acad. Sci. USA 81:6851 (1984)), or by covalently joining to the immunoglobulin coding sequence all or part of the coding sequence for a non-immunoglobulin polypeptide.

Typically, such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

In some embodiments of any of the methods described herein, the antibody is IgA, IgD, IgE, IgG, or IgM. In some embodiments, the antibody is an IgG monoclonal antibody.

### Humanized Antibodies

In some embodiments, the antibody is a humanized antibody. Methods for humanizing non-human antibodies have been described in the art. In some embodiments, a humanized antibody has one or more amino acid residues introduced into it from a source that is non-human. These non-human amino acid residues are often referred to as “import” residues, which are typically taken from an “import” variable domain. Humanization can be essentially performed following the method of Winter and co-workers (Jones et al., Nature 321:522-525 (1986); Riechmann et al., Nature 332:323-327 (1988); Verhoeyen et al., Science 239:1534-1536 (1988)), by substituting hypervariable region sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some hypervariable region residues and possibly some FR residues are substituted by residues from analogous sites in rodent antibodies.

The choice of human variable domains, both light and heavy, to be used in making the humanized antibodies is very important to reduce antigenicity According to the so-called “best-fit” method, the sequence of the variable domain of a rodent antibody is screened against the entire library of known human variable-domain sequences. The human sequence that is closest to that of the rodent is then accepted as the human framework region (FR) for the humanized antibody (Sims et al., J. Immunol. 151:2296 (1993); Chothia et al., J. Mol. Biol. 196:901 (1987)). Another method uses a particular framework region derived from the consensus sequence of all human antibodies of a particular subgroup of light or heavy chain variable regions. The same framework may be used for several different humanized antibodies (Carter et al., Proc. Natl. Acad. Sci. USA 89:4285 (1992); Presta et al., J. Immunol. 151:2623 (1993)).

It is further important that antibodies be humanized with retention of high affinity for the antigen and other favorable biological properties. To achieve this goal, in some embodiments of the methods, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available that illustrate and display probable three-dimensional conformational structures of selected candidate immunoglobulin sequences. Inspection of these displays permits analysis of the likely role of the residues in the functioning of the candidate immunoglobulin sequence. i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

### Human Antibodies

In some embodiments, the antibody is a human antibody. As an alternative to humanization, human antibodies can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al, Proc. Natl. Acad Sci. USA 90:2551 (1993); Jakobovits et al., Nature 362:255-258 (1993); Bruggermann et al. Year in Immuno. 7:33 (1993); and U.S. Pat. Nos. 5,591,669; 5,589,369; and 5,545,807.

Alternatively, phage display technology (McCafferty et al., Nature 348:552-553 (1990)) can be used to produce human antibodies and antibody fragments n vitro, from immunoglobulin variable (V) domain gene repertoires from unimmunized donors. According to this technique, antibody V domain genes are cloned in-frame into either a major or minor coat polypeptide gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B cell. Phage display can be performed in a variety of formats; for their review see, e.g., Johnson, Kevin S. and Chiswell, David J., Current Opinion in Structural Biology 3:564-571 (1993). Several sources of V-gene segments can be used for phage display. Clackson et al., Nature 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al., J. Mol. Biol. 222:581-597 (1991), or Griffith et al., EMBO J. 12-725-734 (1993). See also, U.S. Pat. Nos. 5,565,332 and 5,573,905.

Human antibodies may also be generated by in vitro activated B cells (see U.S. Pat. Nos. 5,567,610 and 5,229,275).

### Antibody Fragments

In some embodiments, the antibody is an antibody fragment. Various techniques have been developed for the production of antibody fragments. Traditionally, these fragments were derived via proteolytic digestion of intact antibodies (see, e.g., Morimoto et al., Journal of Biochemical and Biophysical Methods 24:107-117 (1992) and Brennan et al., Science 229:81 (1985)). However, these fragments can now be produced directly by recombinant host cells. For example, the antibody fragments can be isolated from the antibody phage libraries discussed above. Alternatively. Fab′-SH fragments can be directly recovered from E. coli and chemically coupled to form F(ab′)₂ fragments (Carter et al., Bio/Technology 10:163-167 (1992)). According to another approach, F(ab′)₂ fragments can be isolated directly from recombinant host cell culture. Other techniques for the production of antibody fragments will be apparent to the skilled practitioner. In other embodiments, the antibody of choice is a single chain Fv fragment (scFv). See WO 93/16185; U.S. Pat. Nos. 5,571,894; and 5,587,458. The antibody fragment may also be a “linear antibody,” e.g., as described in U.S. Pat. No. 5,641,870 for example. Such linear antibody fragments may be monospecific or bispecific.

In some embodiments, fragments of the antibodies described herein are provided. In some embodiments, the antibody fragment is an antigen binding fragment. In some embodiments, the antigen binding fragment is selected from the group consisting of a Fab fragment, a Fab′ fragment, a F(ab′)₂ fragment, a scFv, a di-scFv, a bi-scFv, a tandem (di, tri)-scFv, a Fv, a sdAb, a tri-functional antibody, a BiTE, a diabody and a triabody.

### Bispecific Antibodies

In some embodiments, the antibody is a bispecific antibody. Bispecific antibodies are antibodies that have binding specificities for at least two different epitopes. Exemplary bispecific antibodies may bind to two different epitopes. Alternatively, a bispecific antibody binding arm may be combined with an arm that binds to a triggering molecule on a leukocyte such as a T-cell receptor molecule (e.g. CD2 or CD3), or Fc receptors for IgG (FcγR), such as FcγRI (CD64), FcγRII (CD32) and FcγRIII (CD16) so as to focus cellular defense mechanisms to the cell. Bispecific antibodies can be prepared as full-length antibodies or antibody fragments (e.g. F(ab′)₂ bispecific antibodies).

Methods for making bispecific antibodies are known in the art. Traditional production of full length bispecific antibodies is based on the coexpression of two immunoglobulin heavy chain-light chain pairs, where the two chains have different specificities (Millstein et al., Nature 305:537-539 (1983)). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al., EMBO J., 10:3655-3659 (1991).

According to a different approach, antibody variable domains with the desired binding specificities (antibody-antigen combining sites) are fused to immunoglobulin constant domain sequences. In some embodiments, the fusion is with an immunoglobulin heavy chain constant domain, comprising at least part of the hinge, CH2, and CH3 regions. In some embodiments, the first heavy chain constant region (CH1) containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In some embodiments of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et al., Methods in Enzymology 121:210 (1986).

According to another approach described in U.S. Pat. No. 5,731,168, the interface between a pair of antibody molecules can be engineered to maximize the percentage of heterodimers that are recovered from recombinant cell culture. In some embodiments, the interface comprises at least a part of the C_{H}3 domain of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan et al., Science 229: 81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Various techniques for making and isolating bispecific antibody fragments directly from recombinant cell culture have also been described. For example, bispecific antibodies have been produced using leucine zippers. Kostelny et al., J. Immunol. 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad Sci. USA 90:6444-6448 (1993) has provided an alternative mechanism for making bispecific antibody fragments. The fragments comprise a heavy chain variable domain (V_{H}) connected to a light chain variable domain (V_{L}) by a linker that is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See Gruber et al., J. Immunol. 152:5368 (1994).

Antibodies with more than two valencies are contemplated. For example, trispecific antibodies can be prepared. Tutt et al., J. Immunol. 147: 60 (1991).

### Multivalent Antibodies

In some embodiments, the antibodies are multivalent antibodies. A multivalent antibody may be internalized (and/or catabolized) faster than a bivalent antibody by a cell expressing an antigen to which the antibodies bind. The antibodies provided herein can be multivalent antibodies (which are other than of the IgM class) with three or more antigen binding sites (e.g., tetravalent antibodies), which can be readily produced by recombinant expression of nucleic acid encoding the polypeptide chains of the antibody. The multivalent antibody can comprise a dimerization domain and three or more antigen binding sites. The preferred dimerization domain comprises (or consists of) an Fc region or a hinge region. In this scenario, the antibody will comprise an Fc region and three or more antigen binding sites amino-terminal to the Fc region. The preferred multivalent antibody herein comprises (or consists of) three to about eight, but preferably four, antigen binding sites. The multivalent antibody comprises at least one polypeptide chain (and preferably two polypeptide chains), wherein the polypeptide chain(s) comprise two or more variable domains. For instance, the polypeptide chain(s) may comprise VD1-(X1)n-VD2-(X2) n-Fc, wherein VD1 is a first variable domain, VD2 is a second variable domain, Fc is one polypeptide chain of an Fc region, X1 and X2 represent an amino acid or polypeptide, and n is 0 or 1. For instance, the polypeptide chain(s) may comprise: VH-CH1-flexible linker-VH-CH1-Fc region chain; or VH-CH1-VH-CH1-Fc region chain. The multivalent antibody herein preferably further comprises at least two (and preferably four) light chain variable domain polypeptides. The multivalent antibody herein may, for instance, comprise from about two to about eight light chain variable domain polypeptides. The light chain variable domain polypeptides contemplated here comprise a light chain variable domain and, optionally, further comprise a CL domain.

In some embodiments, the antibody is a multispecific antibody. Example of multispecific antibodies include, but are not limited to, an antibody comprising a heavy chain variable domain (V_{H}) and a light chain variable domain (V_{L}), where the V_{H}V_{L} unit has polyepitopic specificity, antibodies having two or more V_{L} and V_{H} domains with each V_{H}V_{L} unit binding to a different epitope, antibodies having two or more single variable domains with each single variable domain binding to a different epitope, full length antibodies, antibody fragments such as Fab, Fv, dsFv, scFv, diabodies, bispecific diabodies, triabodies, tri-functional antibodies, antibody fragments that have been linked covalently or non-covalently. In some embodiment that antibody has polyepitopic specificity; for example, the ability to specifically bind to two or more different epitopes on the same or different target(s). In some embodiments, the antibodies are monospecific; for example, an antibody that binds only one epitope According to one embodiment, the multispecific antibody is an IgG antibody that binds to each epitope with an affinity of 5 μM to 0.001 pM, 3 μM to 0.001 pM, 1 μM to 0.001 pM, 0.5 μM to 0.001 pM, or 0.1 μM to 0.001 pM.

### Other Antibody Modifications

It may be desirable to modify the antibody provided herein with respect to effector function, e.g., so as to enhance antigen-dependent cell-mediated cytotoxicity (ADCC) and/or complement dependent cytotoxicity (CDC) of the antibody. This may be achieved by introducing one or more amino acid substitutions in an Fc region of the antibody. Alternatively or additionally, cysteine residue(s) may be introduced in the Fc region, thereby allowing interchain disulfide bond formation in this region. The homodimeric antibody thus generated may have improved internalization capability and/or increased complement-mediated cell killing and antibody-dependent cellular cytotoxicity (ADCC). See Caron et al., J. Exp Med 176:1191-1195 (1992) and Shopes, B. J., Immunol. 148:2918-2922 (1992). Homodimeric antibodies with enhanced anti-tumor activity may also be prepared using heterobifunctional cross-linkers as described in Wolff et al., Cancer Research 53:2560-2565 (1993). Alternatively, an antibody can be engineered which has dual Fc regions and may thereby have enhanced complement mediated lysis and ADCC capabilities. See Stevenson et al., Anti-Cancer Drug Design 3:219-230 (1989).

For increasing the serum half-life of the antibody, amino acid alterations can be made in the antibody as described in US 2006/0067930, which is hereby incorporated by reference in its entirety.

### Polypeptide Variants and Modifications

Amino acid sequence modification(s) of the polypeptides, including antibodies, described herein may be used in reusable chromatography material cleaned by the methods of described herein.

### Variant Polypeptides

“Polypeptide variant” means a polypeptide, preferably an active polypeptide, as defined herein having at least about 80% amino acid sequence identity with a full-length native sequence of the polypeptide, a polypeptide sequence lacking the signal peptide, an extracellular domain of a polypeptide, with or without the signal peptide. Such polypeptide variants include, for instance, polypeptides wherein one or more amino acid residues are added, or deleted, at the N or C-terminus of the full-length native amino acid sequence. Ordinarily, a TAT polypeptide variant will have at least about 80% amino acid sequence identity, alternatively at least about any of 85%, 90%, 95%, 96%, 97%, 98%, or 99% amino acid sequence identity, to a full-length native sequence polypeptide sequence, a polypeptide sequence lacking the signal peptide, an extracellular domain of a polypeptide, with or without the signal peptide. Optionally, variant polypeptides will have no more than one conservative amino acid substitution as compared to the native polypeptide sequence, alternatively no more than about any of 2, 3, 4, 5, 6, 7, 8, 9, or 10 conservative amino acid substitutions as compared to the native polypeptide sequence.

The variant polypeptide may be truncated at the N-terminus or C-terminus, or may lack internal residues, for example, when compared with a full length native polypeptide. Certain variant polypeptides may lack amino acid residues that are not essential for a desired biological activity. These variant polypeptides with truncations, deletions, and insertions may be prepared by any of a number of conventional techniques. Desired variant polypeptides may be chemically synthesized. Another suitable technique involves isolating and amplifying a nucleic acid fragment encoding a desired variant polypeptide, by polymerase chain reaction (PCR). Oligonucleotides that define the desired termini of the nucleic acid fragment are employed at the 5′ and 3′ primers in the PCR. Preferably, variant polypeptides share at least one biological and/or immunological activity with the native polypeptide disclosed herein.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue or the antibody fused to a cytotoxic polypeptide. Other insertional variants of the antibody molecule include the fusion to the N- or C-terminus of the antibody to an enzyme or a polypeptide which increases the serum half-life of the antibody.

For example, it may be desirable to improve the binding affinity and/or other biological properties of the polypeptide. Amino acid sequence variants of the polypeptide are prepared by introducing appropriate nucleotide changes into the antibody nucleic acid, or by peptide synthesis. Such modifications include, for example, deletions from, and/or insertions into and/or substitutions of, residues within the amino acid sequences of the polypeptide. Any combination of deletion, insertion, and substitution is made to arrive at the final construct, provided that the final construct possesses the desired characteristics. The amino acid changes also may alter post-translational processes of the polypeptide (e.g., antibody), such as changing the number or position of glycosylation sites.

Guidance in determining which amino acid residue may be inserted, substituted or deleted without adversely affecting the desired activity may be found by comparing the sequence of the polypeptide with that of homologous known polypeptide molecules and minimizing the number of amino acid sequence changes made in regions of high homology.

A useful method for identification of certain residues or regions of the polypeptide (e.g., antibody) that are preferred locations for mutagenesis is called “alanine scanning mutagenesis” as described by Cunningham and Wells, Science 244:1081-1085 (1989). Here, a residue or group of target residues are identified (e.g., charged residues such as Arg. Asp, His, Lys, and Glu) and replaced by a neutral or negatively charged amino acid (most preferably alanine or polyalanine) to affect the interaction of the amino acids with antigen Those amino acid locations demonstrating functional sensitivity to the substitutions then are refined by introducing further or other variants at, or for, the sites of substitution Thus, while the site for introducing an amino acid sequence variation is predetermined, the nature of the mutation per se need not be predetermined. For example, to analyze the performance of a mutation at a given site, ala scanning or random mutagenesis is conducted at the target codon or region and the expressed antibody variants are screened for the desired activity.

Another type of variant is an amino acid substitution variant. These variants have at least one amino acid residue in the antibody molecule replaced by a different residue. The sites of greatest interest for substitutional mutagenesis include the hypervariable regions, but FR alterations are also contemplated. Conservative substitutions are shown in the Table 1 below under the heading of “preferred substitutions.” If such substitutions result in a change in biological activity, then more substantial changes, denominated “exemplary substitutions” in the Table 1, or as further described below in reference to amino acid classes, may be introduced and the products screened.

TABLE 1
Exemplary amino acid substitutions

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala, Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Substantial modifications in the biological properties of the polypeptide are accomplished by selecting substitutions that differ significantly in their effect on maintaining (a) the structure of the polypeptide backbone in the area of the substitution, for example, as a sheet or helical conformation. (b) the charge or hydrophobicity of the molecule at the target site, or (c) the bulk of the side chain. Amino acids may be grouped according to similarities in the properties of their side chains (in A. L. Lehninger, Biochemistry second ed., pp. 73-75, Worth Publishers, New York (1975)):

(1) non-polar: Ala (A), Val (V), Leu (L), Ile (I), Pro (P), Phe (F), Trp (W), Met (M)

(2) uncharged polar: Gly (G), Ser (S), Thr (T), Cys (C), Tyr (Y), Asn (N), Gln (Q)

(3) acidic: Asp (D), Glu (E)

(4) basic: Lys (K), Arg (R), His (H)

Alternatively, naturally occurring residues may be divided into groups based on common side-chain properties:

(1) hydrophobic: Norleucine, Met, Ala, Val, Leu, Ile;

(2) neutral hydrophilic: Cys, Ser, Thr, Asn, Gln;

(3) acidic: Asp, Glu;

(4) basic: His, Lys, Arg;

(5) residues that influence chain orientation: Gly, Pro;

(6) aromatic: Trp, Tyr, Phe.

Non-conservative substitutions will entail exchanging a member of one of these classes for another class.

Any cysteine residue not involved in maintaining the proper conformation of the antibody also may be substituted, generally with serine, to improve the oxidative stability of the molecule and prevent aberrant crosslinking. Conversely, cysteine bond(s) may be added to the polypeptide to improve its stability (particularly where the antibody is an antibody fragment such as an Fv fragment).

A particularly preferred type of substitutional variant involves substituting one or more hypervariable region residues of a parent antibody (e.g., a humanized antibody). Generally, the resulting variant(s) selected for further development will have improved biological properties relative to the parent antibody from which they are generated. A convenient way for generating such substitutional variants involves affinity maturation using phage display. Briefly, several hypervariable region sites (e.g., 6-7 sites) are mutated to generate all possible amino substitutions at each site. The antibody variants thus generated are displayed in a monovalent fashion from filamentous phage particles as fusions to the gene III product of M13 packaged within each particle. The phage-displayed variants are then screened for their biological activity (e.g., binding affinity) as herein disclosed. In order to identify candidate hypervariable region sites for modification, alanine scanning mutagenesis can be performed to identify hypervariable region residues contributing significantly to antigen binding. Alternatively, or additionally, it may be beneficial to analyze a crystal structure of the antigen-antibody complex to identify contact points between the antibody and target. Such contact residues and neighboring residues are candidates for substitution according to the techniques elaborated herein. Once such variants are generated, the panel of variants is subjected to screening as described herein and antibodies with superior properties in one or more relevant assays may be selected for further development.

Another type of amino acid variant of the polypeptide alters the original glycosylation pattern of the antibody. The polypeptide may comprise non-amino acid moieties. For example, the polypeptide may be glycosylated. Such glycosylation may occur naturally during expression of the polypeptide in the host cell or host organism, or may be a deliberate modification arising from human intervention. By altering is meant deleting one or more carbohydrate moieties found in the polypeptide, and/or adding one or more glycosylation sites that are not present in the polypeptide.

Glycosylation of polypeptide is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition of glycosylation sites to the polypeptide is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

Removal of carbohydrate moieties present on the polypeptide may be accomplished chemically or enzymatically or by mutational substitution of codons encoding for amino acid residues that serve as targets for glycosylation. Enzymatic cleavage of carbohydrate moieties on polypeptides can be achieved by the use of a variety of endo- and exo-glycosidases.

Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the α-amino groups of lysine, arginine, and histidine side chains, acetylation of the N-terminal amine, and amidation of any C-terminal carboxyl group.

### Chimeric Polypeptides

The polypeptide described herein may be modified in a way to form chimeric molecules comprising the polypeptide fused to another, heterologous polypeptide or amino acid sequence. In some embodiments, a chimeric molecule comprises a fusion of the polypeptide with a tag polypeptide which provides an epitope to which an anti-tag antibody can selectively bind. The epitope tag is generally placed at the amino- or carboxyl-terminus of the polypeptide. The presence of such epitope-tagged forms of the polypeptide can be detected using an antibody against the tag polypeptide. Also, provision of the epitope tag enables the polypeptide to be readily purified by affinity purification using an anti-tag antibody or another type of affinity matrix that binds to the epitope tag.

In an alternative embodiment, the chimeric molecule may comprise a fusion of the polypeptide with an immunoglobulin or a particular region of an immunoglobulin. A bivalent form of the chimeric molecule is referred to as an “immunoadhesin.”

As used herein, the term “immunoadhesin” designates antibody-like molecules which combine the binding specificity of a heterologous polypeptide with the effector functions of immunoglobulin constant domains. Structurally, the immunoadhesins comprise a fusion of an amino acid sequence with the desired binding specificity which is other than the antigen recognition and binding site of an antibody (i.e., is “heterologous”), and an immunoglobulin constant domain sequence. The adhesin part of an immunoadhesin molecule typically is a contiguous amino acid sequence comprising at least the binding site of a receptor or a ligand. The immunoglobulin constant domain sequence in the immunoadhesin may be obtained from any immunoglobulin, such as IgG-1, IgG-2, IgG-3, or IgG4 subtypes, IgA (including IgA-1 and IgA-2), IgE, IgD or IgM.

The Ig fusions preferably include the substitution of a soluble (transmembrane domain deleted or inactivated) form of a polypeptide in place of at least one variable region within an Ig molecule. In a particularly preferred embodiment, the immunoglobulin fusion includes the hinge, CH₂ and CH₃, or the hinge, CH₁, CH₂ and CH₃ regions of an IgG1 molecule.

### Polypeptide Conjugates

The polypeptide for use in polypeptide formulations may be conjugated to a cytotoxic agent such as a chemotherapeutic agent, a growth inhibitory agent, a toxin (e.g., an enzymatically active toxin of bacterial, fungal, plant, or animal origin, or fragments thereof), or a radioactive isotope (i.e., a radioconjugate).

Chemotherapeutic agents useful in the generation of such conjugates can be used. In addition, enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes. A variety of radionuclides are available for the production of radioconjugated polypeptides. Examples include ²¹²Bi, m¹³¹I, ¹¹³In, ⁹⁰Y, and ¹⁸⁶Re. Conjugates of the polypeptide and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis(p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta el al., Science 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the polypeptide.

Conjugates of a polypeptide and one or more small molecule toxins, such as a calicheamicin, maytansinoids, a trichothene, and CC1065, and the derivatives of these toxins that have toxin activity, are also contemplated herein.

Maytansinoids are mitototic inhibitors which act by inhibiting tubulin polymerization. Maytansine was first isolated from the east African shrub Maytenus serrata. Subsequently, it was discovered that certain microbes also produce maytansinoids, such as maytansinol and C-3 maytansinol esters. Synthetic maytansinol and derivatives and analogues thereof are also contemplated. There are many linking groups known in the art for making polypeptide-maytansinoid conjugates, including, for example, those disclosed in U.S. Pat. No. 5,208,020. The linking groups include disufide groups, thioether groups, acid labile groups, photolabile groups, peptidase labile groups, or esterase labile groups, as disclosed in the above-identified patents, disulfide and thioether groups being preferred.

The linker may be attached to the maytansinoid molecule at various positions, depending on the type of the link. For example, an ester linkage may be formed by reaction with a hydroxyl group using conventional coupling techniques. The reaction may occur at the C-3 position having a hydroxyl group, the C-14 position modified with hydroxymethyl, the C-15 position modified with a hydroxyl group, and the C-20 position having a hydroxyl group. In a preferred embodiment, the linkage is formed at the C-3 position of maytansinol or a maytansinol analogue.

Another conjugate of interest comprises a polypeptide conjugated to one or more calicheamicin molecules. The calicheamicin family of antibiotics are capable of producing double-stranded DNA breaks at sub-picomolar concentrations. For the preparation of conjugates of the calicheamicin family, see, e.g., U.S. Pat. No. 5,712,374. Structural analogues of calicheamicin which may be used include, but are not limited to, γ₁¹, α₂¹, α₃¹, N-acetyl-γ₁¹, PSAG and θ₁¹. Another anti-tumor drug that the antibody can be conjugated is QFA which is an antifolate. Both calicheamicin and QFA have intracellular sites of action and do not readily cross the plasma membrane. Therefore, cellular uptake of these agents through polypeptide (e.g., antibody) mediated internalization greatly enhances their cytotoxic effects.

Other antitumor agents that can be conjugated to the polypeptides described herein include BCNU, streptozoicin, vincristine and 5-fluorouracil, the family of agents known collectively LL-E33288 complex, as well as esperamicins.

In some embodiments, the polypeptide may be a conjugate between a polypeptide and a compound with nucleolytic activity (e.g., a ribonuclease or a DNA endonuclease such as a deoxyribonuclease; DNase).

In yet another embodiment, the polypeptide (e.g., antibody) may be conjugated to a “receptor” (such streptavidin) for utilization in tumor pre-targeting wherein the polypeptide receptor conjugate is administered to the patient, followed by removal of unbound conjugate from the circulation using a clearing agent and then administration of a “ligand” (e.g., avidin) which is conjugated to a cytotoxic agent (e.g., a radionucleotide).

In some embodiments, the polypeptide may be conjugated to a prodrug-activating enzyme which converts a prodrug (e.g., a peptidyl chemotherapeutic agent) to an active anti-cancer drug. The enzyme component of the immunoconjugate includes any enzyme capable of acting on a prodrug in such a way so as to convert it into its more active, cytotoxic form.

Enzymes that are useful include, but are not limited to, alkaline phosphatase useful for converting phosphate-containing prodrugs into free drugs; arylsulfatase useful for converting sulfate-containing prodrugs into free drugs; cytosine deaminase useful for converting non-toxic 5-fluorocytosine into the anti-cancer drug, 5-fluorouracil; proteases, such as serratia protease, thermolysin, subtilisin, carboxypeptidases and cathepsins (such as cathepsins B and L), that are useful for converting peptide-containing prodrugs into free drugs; D-alanylcarboxypeptidases, useful for converting prodrugs that contain D-amino acid substituents; carbohydrate-cleaving enzymes such as β-galactosidase and neuraminidase useful for converting glycosylated prodrugs into free drugs; β-lactamase useful for converting drugs derivatized with β-lactams into free drugs; and penicillin amidases, such as penicillin V amidase or penicillin G amidase, useful for converting drugs derivatized at their amine nitrogens with phenoxyacetyl or phenylacetyl groups, respectively, into free drugs. Alternatively, antibodies with enzymatic activity, also known in the art as “abzymes”, can be used to convert the prodrugs into free active drugs.

### Other

Another type of covalent modification of the polypeptide comprises linking the polypeptide to one of a variety of nonproteinaceous polymers, e.g., polyethylene glycol, polypropylene glycol, polyoxyalkylenes, or copolymers of polyethylene glycol and polypropylene glycol. The polypeptide also may be entrapped in microcapsules prepared, for example, by coacervation techniques or by interfacial polymerization (for example, hydroxymethylcellulose or gelatin-microcapsules and poly-(methylmethacylate) microcapsules, respectively), in colloidal drug delivery systems (for example, liposomes, albumin microspheres, microemulsions, nano-particles and nanocapsules), or in macroemulsions. Such techniques are disclosed in Remington's Pharmaceutical Sciences, 18th edition, Gennaro, A. R., Ed., (1990).

### Obtaining Polypeptides for Use in the Formulations and Methods

The polypeptides to be purified using reusable chromatography material cleaned by the methods described herein may be obtained using methods well-known in the art, including the recombination methods. The following sections provide guidance regarding these methods.

### Polynucleotides

“Polynucleotide,” or “nucleic acid,” as used interchangeably herein, refer to polymers of nucleotides ofany length, and include DNA and RNA.

Polynucleotides encoding polypeptides may be obtained from any source including, but not limited to, a cDNA library prepared from tissue believed to possess the polypeptide mRNA and to express it at a detectable level. Accordingly, polynucleotides encoding polypeptide can be conveniently obtained from a cDNA library prepared from human tissue. The polypeptide-encoding gene may also be obtained from a genomic library or by known synthetic procedures (e.g., automated nucleic acid synthesis).

For example, the polynucleotide may encode an entire immunoglobulin molecule chain, such as a light chain or a heavy chain. A complete heavy chain includes not only a heavy chain variable region (V_{H}) but also a heavy chain constant region (C_{H}), which typically will comprise three constant domains: C_{H}1, C_{H}2 and C_{H}3; and a “hinge” region. In some situations, the presence of a constant region is desirable.

Other polypeptides which may be encoded by the polynucleotide include antigen-binding antibody fragments such as single domain antibodies (“dAbs”), Fv, scFv, Fab′ and F(ab′)₂ and “minibodies.” Minibodies are (typically) bivalent antibody fragments from which the C_{H}1 and C_{K} or C_{L} domain has been excised. As minibodies are smaller than conventional antibodies they should achieve better tissue penetration in clinical/diagnostic use, but being bivalent they should retain higher binding affinity than monovalent antibody fragments, such as dAbs. Accordingly, unless the context dictates otherwise, the term “antibody” as used herein encompasses not only whole antibody molecules but also antigen-binding antibody fragments of the type discussed above. Preferably each framework region present in the encoded polypeptide will comprise at least one amino acid substitution relative to the corresponding human acceptor framework Thus, for example, the framework regions may comprise, in total, three, four, five, six, seven, eight, nine, ten, eleven, twelve, thirteen, fourteen, or fifteen amino acid substitutions relative to the acceptor framework regions.

Suitably, the polynucleotides described herein may be isolated and/or purified. In some embodiments, the polynucleotides are isolated polynucleotides.

The term “isolated polynucleotide” is intended to indicate that the molecule is removed or separated from its normal or natural environment or has been produced in such a way that it is not present in its normal or natural environment. In some embodiments, the polynucleotides are purified polynucleotides. The term purified is intended to indicate that at least some contaminating molecules or substances have been removed.

Suitably, the polynucleotides are substantially purified, such that the relevant polynucleotides constitute the dominant (i.e., most abundant) polynucleotides present in a composition.

### Expression of Polynucleotides

The description below relates primarily to production of polypeptides by culturing cells transformed or transfected with a vector containing polypeptide-encoding polynucleotides. It is, of course, contemplated that alternative methods, which are well known in the art, may be employed to prepare polypeptides. For instance, the appropriate amino acid sequence, or portions thereof, may be produced by direct peptide synthesis using solid-phase techniques (see, e.g., Stewart et al., Solid-Phase Peptide Synthesis W.H. Freeman Co., San Francisco, Calif. (1969); Merrifield, J. Am. Chem. Soc. 85:2149-2154 (1963)). In vitro protein synthesis may be performed using manual techniques or by automation. Automated synthesis may be accomplished, for instance, using an Applied Biosystems Peptide Synthesizer (Foster City, Calif.) using manufacturer's instructions. Various portions of the polypeptide may be chemically synthesized separately and combined using chemical or enzymatic methods to produce the desired polypeptide.

Polynucleotides as described herein are inserted into an expression vector(s) for production of the polypeptides. The term “control sequences” refers to DNA sequences necessary for the expression of an operably linked coding sequence in a particular host organism. The control sequences include, but are not limited to, promoters (e.g., naturally-associated or heterologous promoters), signal sequences, enhancer elements, and transcription termination sequences.

A polynucleotide is “operably linked” when it is placed into a functional relationship with another polynucleotide sequence. For example, nucleic acids for a presequence or secretory leader is operably linked to nucleic acids for a polypeptide if it is expressed as a preprotein that participates in the secretion of the polypeptide; a promoter or enhancer is operably linked to a coding sequence if it affects the transcription of the sequence, or a ribosome binding site is operably linked to a coding sequence if it is positioned so as to facilitate translation. Generally, “operably linked” means that the nucleic acid sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

For antibodies, the light and heavy chains can be cloned in the same or different expression vectors. The nucleic acid segments encoding immunoglobulin chains are operably linked to control sequences in the expression vector(s) that ensure the expression of immunoglobulin polypeptides.

The vectors containing the polynucleotide sequences (e.g., the variable heavy and/or variable light chain encoding sequences and optional expression control sequences) can be transferred into a host cell by well-known methods, which vary depending on the type of cellular host. For example, calcium chloride transfection is commonly utilized for prokaryotic cells, whereas calcium phosphate treatment, electroporation, lipofection, biolistics or viral-based transfection may be used for other cellular hosts. (See generally Sambrook et al., Molecular Cloning: A Laboratory Manual (Cold Spring Harbor Press, 2nd ed., 1989). Other methods used to transform mammalian cells include the use of polybrene, protoplast fusion, liposomes, electroporation, and microinjection. For production of transgenic animals, transgenes can be microinjected into fertilized oocytes, or can be incorporated into the genome of embryonic stem cells, and the nuclei of such cells transferred into enucleated oocytes.

### Vectors

The term “vector” includes expression vectors and transformation vectors and shuttle vectors.

The term “expression vector” means a construct capable of in vivo or in vitro expression.

The term “transformation vector” means a construct capable of being transferred from one entity to another entity—which may be of the species or may be of a different species. If the construct is capable of being transferred from one species to another—such as from an Escherichia coli plasmid to a bacterium, such as of the genus Bacillus, then the transformation vector is sometimes called a “shuttle vector”. It may even be a construct capable of being transferred from an E. coli plasmid to an Agrobacterium to a plant.

Vectors may be transformed into a suitable host cell as described below to provide for expression of a polypeptide. Various vectors are publicly available. The vector may, for example, be in the form of a plasmid, cosmid, viral particle, or phage. The appropriate nucleic acid sequence may be inserted into the vector by a variety of procedures. In general, DNA is inserted into an appropriate restriction endonuclease site(s) using techniques known in the art. Construction of suitable vectors containing one or more of these components employs standard ligation techniques which are known to the skilled artisan.

The vectors may be for example, plasmid, virus or phage vectors provided with an origin of replication, optionally a promoter for the expression of the said polynucleotide and optionally a regulator of the promoter. Vectors may contain one or more selectable marker genes which are well known in the art.

These expression vectors are typically replicable in the host organisms either as episomes or as an integral part of the host chromosomal DNA.

### Host Cells

The host cell may be a bacterium, a yeast or other fungal cell, insect cell, a plant cell, or a mammalian cell, for example.

A transgenic multicellular host organism which has been genetically manipulated may be used to produce a polypeptide. The organism may be, for example, a transgenic mammalian organism (e.g., a transgenic goat or mouse line).

Suitable prokaryotes include but are not limited to eubacteria, such as Gram-negative or Gram-positive organisms, for example, Enterobacteriaceae such as E. coli. Various E. coli strains are publicly available, such as E coli K12 strain MM294 (ATCC 31,446); E. coli X1776 (ATCC 31,537); E. coli strain W3110 (ATCC 27.325) and K5 772 (ATCC 53,635). Other suitable prokaryotic host cells include Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P), Pseudomonas such as P. aeruginosa, and Streptomyces. These examples are illustrative rather than limiting. Strain W3110 is one particularly preferred host or parent host because it is a common host strain for recombinant polynucleotide product fermentations. Preferably, the host cell secretes minimal amounts of proteolytic enzymes. For example, strain W3110 may be modified to effect a genetic mutation in the genes encoding polypeptides endogenous to the host, with examples of such hosts including E. coli W3110 strain 1A2, which has the complete genotype tonA; E. coli W3110 strain 9E4, which has the complete genotype tonA ptr3; E. coli W3110 strain 27C7 (ATCC 55,244), which has the complete genotype tonA ptr3 phoA E15 (argF-lac)169 degP ompT kan; E. coli W3110 strain 37D6, which has the complete genotype tonA ptr3 phoA E15 (argF-lac)169 degP ompT rbs7 ilvG kan, E. coli W3110 strain 40B4, which is strain 37D6 with a non-kanamycin resistant degP deletion mutation; and an E. coli strain having mutant periplasmic protease. Alternatively, in vitro methods of cloning. e.g., PCR or other nucleic acid polymerase reactions, are suitable.

In these prokaryotic hosts, one can make expression vectors, which will typically contain expression control sequences compatible with the host cell (e.g., an origin of replication). In addition, any number of a variety of well-known promoters will be present, such as the lactose promoter system, a tryptophan (trp) promoter system, a beta-lactamase promoter system, or a promoter system from phage lambda. The promoters will typically control expression, optionally with an operator sequence, and have ribosome binding site sequences and the like, for initiating and completing transcription and translation.

Eukaryotic microbes may be used for expression. Eukaryotic microbes such as filamentous fungi or yeast are suitable cloning or expression hosts for polypeptide-encoding vectors. Saccharomyces cerevisiae is a commonly used lower eukaryotic host microorganism. Others include Schizosaccharomyces pombe; Kluyveromyces hosts such as, e.g., K. lactis (MW98-8C, CBS683, CBS4574), K. fragilis (ATCC 12.424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris; Candida; Trichoderma reesia; Neurospora crassa; Schwannomyces such as Schwannomyces occidentalis; and filamentous fungi such as, e.g., Neurospora, Penicillium, Tolypocladium, and Aspergillus hosts such as A. nidulans, and A. niger. Methylotropic yeasts are suitable herein and include, but are not limited to, yeast capable of growth on methanol selected from the genera consisting of Hansenula, Candida, Kloeckera, Pichia, Saccharomyces, Torulopsis, and Rhodotorula, Saccharomyces is a preferred yeast host, with suitable vectors having expression control sequences (e.g., promoters), an origin of replication, termination sequences and the like as desired. Typical promoters include 3-phosphoglycerate kinase and other glycolytic enzymes. Inducible yeast promoters include, among others, promoters from alcohol dehydrogenase, isocytochrome C, and enzymes responsible for maltose and galactose utilization.

In addition to microorganisms, mammalian tissue cell culture may also be used to express and produce the polypeptides as described herein and in some instances are preferred (See Winnacker, From Genes to Clones VCH Publishers, N.Y., N.Y. (1987). For some embodiments, eukaryotic cells may be preferred, because a number of suitable host cell lines capable of secreting heterologous polypeptides (e.g., intact immunoglobulins) have been developed in the art, and include CHO cell lines, various Cos cell lines, HeLa cells, preferably, myeloma cell lines, or transformed B-cells or hybridomas. In some embodiments, the mammalian host cell is a CHO cell.

In some embodiments, the host cell is a vertebrate host cell Examples of useful mammalian host cell lines are monkey kidney CV1 line transformed by SV40 (COS-7, ATCC CRL 1651); human embryonic kidney line (293 or 293 cells subcloned for growth in suspension culture); baby hamster kidney cells (BHK, ATCC CCL 10); Chinese hamster ovary cells/-DHFR(CHO or CHO-DP-12 line); mouse sertoli cells; monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells; MRC 5 cells; FS4 cells; and a human hepatoma line (Hep G2).

### Formulations and Methods of Making of the Formulations

Provided herein are also formulations and methods of making the formulation comprising the polypeptides (e.g., antibodies) purified by the methods described herein. For example, the purified polypeptide may be combined with a pharmaceutically acceptable carrier.

The polypeptide formulations in some embodiments may be prepared for storage by mixing a polypeptide having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions.

“Carriers” as used herein include pharmaceutically acceptable carriers, excipients, or stabilizers which are nontoxic to the cell or manual being exposed thereto at the dosages and concentrations employed. Often the physiologically acceptable carrier is an aqueous pH buffered solution.

Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

In some embodiments, the polypeptide in the polypeptide formulation maintains functional activity.

The formulations to be used for in vivo administration must be sterile. This is readily accomplished by filtration through sterile filtration membranes.

The formulations herein may also contain more than one active compound as necessary for the particular indication being treated, preferably those with complementary activities that do not adversely affect each other. For example, in addition to a polypeptide, it may be desirable to include in the one formulation, an additional polypeptide (e.g., antibody). Alternatively, or additionally, the composition may further comprise a chemotherapeutic agent, cytotoxic agent, cytokine, growth inhibitory agent, anti-hormonal agent, and/or cardioprotectant. Such molecules are suitably present in combination in amounts that are effective for the purpose intended.

### Articles of Manufacture

The polypeptides purified by the methods described herein and/or formulations comprising the polypeptides purified by the methods described herein may be contained within an article of manufacture. The article of manufacture may comprise a container containing the polypeptide and/or the polypeptide formulation. Preferably, the article of manufacture comprises: (a) a container comprising a composition comprising the polypeptide and/or the polypeptide formulation described herein within the container; and (b) a package insert with instructions for administering the formulation to a subject.

The article of manufacture comprises a container and a label or package insert on or associated with the container. Suitable containers include, for example, bottles, vials, syringes, etc. The containers may be formed from a variety of materials such as glass or plastic. The container holds or contains a formulation and may have a sterile access port (for example the container may be an intravenous solution bag or a vial having a stopper pierceable by a hypodermic injection needle). At least one active agent in the composition is the polypeptide. The label or package insert indicates that the composition's use in a subject with specific guidance regarding dosing amounts and intervals of polypeptide and any other drug being provided. The article of manufacture may further include other materials desirable from a commercial and user standpoint, including other buffers, diluents, filters, needles, and syringes. In some embodiments, the container is a syringe. In some embodiments, the syringe is further contained within an injection device. In some embodiments, the injection device is an autoinjector.

A “package insert” is used to refer to instructions customarily included in commercial packages of therapeutic products, that contain information about the indications, usage, dosage, administration, contraindications, other therapeutic products to be combined with the packaged product, and/or warnings concerning the use of such therapeutic products.

### EXEMPLARY EMBODIMENTS

The invention provides the following exemplary embodiments.

Embodiment 1. A method to clean an ion exchange chromatography material for reuse wherein the chromatography material is used in overload mode in the purification of a polypeptide, the method comprising the steps of
a) passing a regeneration buffer through the ion exchange chromatography material, wherein the regeneration buffer comprises sodium acetate (NaOAc);
b) passing of sanitization buffer through the ion exchange chromatography material, wherein the sanitization buffer comprises about 0.5 N NaOH; and
c) storing the ion exchange chromatography material in a storage buffer, wherein the storage buffer comprises about 0.1 N NaOH.

Embodiment 2. The method of embodiment 1, wherein the regeneration buffer comprises about 50 to about 600 mM NaOAc.

Embodiment 3. The method of embodiment 1 or 2, wherein the regeneration buffer comprises about 350 mM NaOAc.

Embodiment 4. The method of any one of embodiments 1-3, wherein the pH of the regeneration buffer is about pH 4.0 to 11.

Embodiment 5. The method of any one of embodiments 1-4, wherein the pH of the regeneration buffer is about pH 8.3.

Embodiment 6. The method of any one of embodiments 1-5, wherein about 3 to about 12 material volumes of regeneration buffer are passed through the ion exchange chromatography material.

Embodiment 7. The method of any one of embodiments 1-6, wherein about 10 material volumes of regeneration buffer are passed through the ion exchange chromatography material.

Embodiment 8. The method of any one of embodiments 1-7, wherein about 2 to about 12 material volumes of sanitization buffer are passed through the ion exchange chromatography material.

Embodiment 9. The method of any one of embodiments 1-8, wherein about 10 material volumes of sanitization buffer are passed passing a regeneration buffer through the ion exchange chromatography material.

Embodiment 10. The method of any one of embodiments 1-9, wherein the ion exchange chromatography material is stored in about 1 to 5 material volumes of storage buffer.

Embodiment 11. The method of any one of embodiments 1-10, wherein the ion exchange chromatography material is stored in about 4 material volumes of storage buffer.

Embodiment 12. A method to clean an ion exchange chromatography material for reuse wherein the chromatography material is used in overload mode in the purification of a polypeptide, the method comprising the steps of
a) passing a regeneration buffer through the ion exchange chromatography material, wherein the regeneration buffer comprises sodium acetate;
b) passing an acid wash through the ion exchange chromatography material,
c) passing of sanitization buffer through the ion exchange chromatography material, wherein the sanitization buffer comprises about 0.5 N NaOH and about 1 M NaCl; and
d) storing the ion exchange chromatography material in a storage buffer, wherein the storage buffer comprises about 0.1 N NaOH.

Embodiment 13. The method of embodiment 12, wherein the regeneration buffer and the second regeneration buffer comprises about 25 to about 600 mM NaOAc.

Embodiment 14. The method of embodiment 12 or 13, wherein the regeneration buffer comprises about 350 mM NaOAc.

Embodiment 15. The method of embodiment 12 or 13, wherein the regeneration buffer comprises about 40 mM NaOAc.

Embodiment 16. The method of any one of embodiments 12-15, wherein the pH of the regeneration buffer is about pH 2.0 to 7.0.

Embodiment 17. The method of any one of embodiments 12-16, wherein the pH of the regeneration buffer is about pH 4.0.

Embodiment 18. The method of any one of embodiments 12-17, wherein about 3 to about 12 material volumes of regeneration buffer are passed through the ion exchange chromatography material.

Embodiment 19. The method of any one of embodiments 12-18, wherein about 7 material volumes of regeneration buffer are passed through the ion exchange chromatography material.

Embodiment 20. The method of any one of embodiments 12-19, wherein the acid wash is about 167M NaOAc and about 0.3M phosphoric acid.

Embodiment 21. The method of any one of embodiments 12-20, wherein about 1 to about 5 material volumes of the acid wash are passed through the ion exchange chromatography material.

Embodiment 22. The method of any one of embodiments 12-21, wherein about 3 material volumes of the acid wash are passed through the ion exchange chromatography material.

Embodiment 23. The method of any one of embodiments 12-22, wherein about 2 to about 12 material volumes of sanitization buffer are passed through the ion exchange chromatography material.

Embodiment 24. The method of any one of embodiments 12-23, wherein about 4 material volumes of sanitization buffer are passed passing a regeneration buffer through the ion exchange chromatography material.

Embodiment 25. The method of any one of embodiments 12-24, wherein the ion exchange chromatography material is stored in about 1 to 5 material volumes of storage buffer.

Embodiment 26. The method of any one of embodiments 12-25, wherein the ion exchange chromatography material is stored in about 3 material volumes of storage buffer.

Embodiment 27. A method to clean an ion exchange chromatography material for reuse wherein the chromatography material is used in overload mode in the purification of a polypeptide, the method comprising the steps of
a) passing a regeneration buffer through the ion exchange chromatography material, wherein the regeneration buffer comprises Tris and sodium acetate;
b) passing a sanitization buffer through the ion exchange chromatography material, wherein the sanitization buffer comprises about 0.5 N NaOH; and
c) storing the ion exchange chromatography material in a storage buffer, wherein the storage buffer comprises about 0.1 N NaOH.

Embodiment 28. The method of embodiment 27, wherein the regeneration buffer comprises about 25 to about 75 mM Ins and about 50 to about 100 mM NaOAc.

Embodiment 29. The method of embodiment 27 or 28, wherein the regeneration buffer comprises about 50 mM Tris and about 85 mM NaOAc.

Embodiment 30. The method of any one of embodiments 27-29, wherein the pH of the regeneration buffer is about pH 7 to 9.

Embodiment 31. The method of any one of embodiments 27-30, wherein the pH of the regeneration buffer is about pH 8.0.

Embodiment 32. The method of any one of embodiments 27-31, wherein about 3 to about 12 material volumes of regeneration buffer are passed through the ion exchange chromatography material.

Embodiment 33. The method of any one of embodiments 27-32, wherein about 10 material volumes of regeneration buffer are passed through the ion exchange chromatography material.

Embodiment 34. The method of any one of embodiments 27-33, wherein about 2 to about 12 material volumes of sanitization buffer are passed through the ion exchange chromatography material.

Embodiment 35. The method of any one of embodiments 27-34, wherein about 10 material volumes of sanitization buffer are passed passing a regeneration buffer through the ion exchange chromatography material.

Embodiment 36. The method of any one of embodiments 27-35, wherein the ion exchange chromatography material is stored in about 1 to 5 material volumes of storage buffer.

Embodiment 37. The method of any one of embodiments 27-36, wherein the ion exchange chromatography material is stored in about 4 material volumes of storage buffer.

Embodiment 38. A method for purifying a polypeptide from a composition comprising the polypeptide and an impurity using an ion exchange chromatography material wherein the ion exchange chromatography material is suitable for reuse, the method comprising the steps of
a) equilibrating an ion exchange chromatography material with an equilibration buffer;
b) loading the composition on the ion exchange chromatography material, wherein the load density is about ≤1000 g/L;
c) collecting fractions comprising the polypeptide;
d) washing the ion exchange chromatography material with equilibration buffer;
e) passing a regeneration buffer through the ion exchange chromatography material, wherein the regeneration buffer comprises sodium acetate;
f) passing of sanitization buffer through the ion exchange chromatography material, wherein the sanitization buffer comprises about 0.5 N NaOH; and
g) storing the ion exchange chromatography material in a storage buffer, wherein the storage buffer comprises about 0.1 N NaOH.

Embodiment 39. A method for purifying a polypeptide from a composition comprising the polypeptide and an impurity using an ion exchange chromatography material wherein the ion exchange chromatography material is suitable for reuse, the method comprising the steps of
a) equilibrating an ion exchange chromatography material with an equilibration buffer;
b) loading the composition on the ion exchange chromatography material, wherein the load density is ≥20 g/L;
c) collecting fractions comprising the polypeptide;
d) washing the ion exchange chromatography material with equilibration buffer;
e) passing a regeneration buffer through the ion exchange chromatography material, wherein the regeneration buffer comprises sodium acetate;
f) passing of sanitization buffer through the ion exchange chromatography material, wherein the sanitization buffer comprises about 0.5 N NaOH; and
g) storing the ion exchange chromatography material in a storage buffer, wherein the storage buffer comprises about 0.1 N NaOH.

Embodiment 40. The method of embodiment 39, wherein the load density is ≥70 g/L.

Embodiment 41. A method for purifying a polypeptide from a composition comprising the polypeptide and an impurity using an ion exchange chromatography material wherein the ion exchange chromatography material is suitable for reuse, the method comprising the step
a) equilibrating an ion exchange chromatography material with an equilibration buffer;
b) loading the composition on the ion exchange chromatography material, wherein the load density exceeds the dynamic binding capacity of the ion exchange chromatography for the polypeptide;
c) collecting fractions comprising the polypeptide;
d) washing the ion exchange chromatography material with equilibration buffer;
e) passing a regeneration buffer through the ion exchange chromatography material, wherein the regeneration buffer comprises sodium acetate;
f) passing of sanitization buffer through the ion exchange chromatography material, wherein the sanitization buffer comprises about 0.5 N NaOH; and
g) storing the ion exchange chromatography material in a storage buffer, wherein the storage buffer comprises about 0.1 N NaOH.

Embodiment 42. The method of embodiment 41, wherein the load density is at least about 2× the dynamic binding capacity of the ion exchange chromatography material for the polypeptide.

Embodiment 43. The method of embodiment 42, wherein the load density is about 2× to about 100× the dynamic binding capacity of the ion exchange chromatography material for the polypeptide.

Embodiment 44. The method of embodiment 42, wherein the load density is about 10× the dynamic binding capacity of the ion exchange chromatography material for the polypeptide.

Embodiment 45. The method of any one of embodiments 38-44, wherein the equilibration buffer comprises about 20 mM to about 60 mM sodium acetate.

Embodiment 46. The method of any one of embodiments 38-45, wherein the equilibration buffer comprises about 40 mM sodium acetate.

Embodiment 47. The method of any one of embodiments 38-46, wherein the pH of the equilibration buffer is about pH 5.0 to about pH 6.0.

Embodiment 48. The method of any one of embodiments 38-47, wherein the pH of the equilibration buffer is about pH 5.5.

Embodiment 49. The method of any one of embodiments 38-48, wherein the ion exchange chromatography material is washed with about 3-7 material volumes of equilibration buffer.

Embodiment 50. The method of any one of embodiments 38-49, wherein the ion exchange chromatography material is washed with about 5 material volumes of equilibration buffer.

Embodiment 51. The method of any one of embodiments 38-50, wherein the regeneration buffer comprises about 50 to about 600 mM NaOAc.

Embodiment 52. The method of any one of embodiments 38-51, wherein the regeneration buffer comprises about 350 mM NaOAc.

Embodiment 53. The method of any one of embodiments 38-52, wherein the pH of the regeneration buffer is about pH 4.0 to 11.

Embodiment 54. The method of any one of embodiments 38-53, wherein the pH of the regeneration buffer is about pH 8.3.

Embodiment 55. The method of any one of embodiments 38-54, wherein about 3 to about 12 material volumes of regeneration buffer are passed through the ion exchange chromatography material.

Embodiment 56. The method of any one of embodiments 38-55, wherein about 10 material volumes of regeneration buffer are passed through the ion exchange chromatography material.

Embodiment 57. The method of any one of embodiments 38-56, wherein about 2 to about 12 material volumes of sanitization buffer are passed through the ion exchange chromatography material.

Embodiment 58. The method of any one of embodiments 38-57, wherein about 10 material volumes of sanitization buffer are passed passing a regeneration buffer through the ion exchange chromatography material.

Embodiment 59. The method of any one of embodiments 38-58, wherein the ion exchange chromatography material is stored in about 1 to 5 material volumes of storage buffer.

Embodiment 60. The method of any one of embodiments 38-59, wherein the ion exchange chromatography material is stored in about 4 material volumes of storage buffer.

Embodiment 61. A method for purifying a polypeptide from a composition comprising the polypeptide and an impurity using an ion exchange chromatography material wherein the ion exchange chromatography material is suitable for reuse, the method comprising the steps of
a) equilibrating an ion exchange chromatography material with an equilibration buffer;
b) loading the composition on the ion exchange chromatography material, wherein the load density exceeds the dynamic binding capacity of the ion exchange chromatography for the polypeptide;
c) collecting fractions comprising the polypeptide;
d) washing the ion exchange chromatography material with equilibration buffer;
e) passing a regeneration buffer through the ion exchange chromatography material, wherein the regeneration buffer comprises sodium acetate;
f) passing an acid wash through the ion exchange chromatography material,
g) passing a sanitization buffer through the ion exchange chromatography material, wherein the sanitization buffer comprises about 0.5 N NaOH and about 1 M NaCl; and
h) storing the ion exchange chromatography material in a storage buffer, wherein the storage buffer comprises about 0.1 N NaOH.

Embodiment 62. The method of embodiment 61, wherein the load density is ≤about 1000 g/L.

Embodiment 63. The method of embodiment 61 or 62, wherein the load density is ≥about 20 g/L.

Embodiment 64. The method of any one of embodiments 61-63, wherein the load density is ≥about 70 g/L.

Embodiment 65. The method of embodiment 61, wherein the load density is at least about 2× the dynamic binding capacity of the ion exchange chromatography material for the polypeptide.

Embodiment 66. The method of embodiment 61, wherein the load density is about 2× to about 100× the dynamic binding capacity of the ion exchange chromatography material for the polypeptide.

Embodiment 67. The method of embodiment 61, wherein the load density is about 10× the dynamic binding capacity of the ion exchange chromatography material for the polypeptide.

Embodiment 68. The method of any one of embodiments 61-67, wherein the equilibration buffer comprises about 50 mM to about 100 mM NaOAc and about 25 mM to about 75 mM Tris.

Embodiment 69. The method of any one of embodiments 61-68, wherein the equilibration buffer comprises about 85 mM sodium acetate and about 50 mM Tris.

Embodiment 70. The method of any one of embodiments 61-69, wherein the pH of the equilibration buffer is about pH 6.0 to about pH 10.0.

Embodiment 71. The method of any one of embodiments 61-70, wherein the pH of the equilibration buffer is about pH 8.0.

Embodiment 72. The method of any one of embodiments 61-71, wherein the ion exchange chromatography material is washed with about 5 to about 15 material volumes of equilibration buffer.

Embodiment 73. The method of any one of embodiments 61-72, wherein the ion exchange chromatography material is washed with about 10 material volumes of equilibration buffer.

Embodiment 74. The method of any one of embodiments 61-73, wherein the regeneration buffer comprises about 25 to about 600 mM NaOAc.

Embodiment 75. The method of any one of embodiments 61-74, wherein the regeneration buffer comprises about 350 mM NaOAc.

Embodiment 76. The method of any one of embodiments 61-74, wherein the regeneration buffer comprises about 40 mM NaOAc.

Embodiment 77. The method of any one of embodiments 61-76, wherein the pH of the regeneration buffer is about pH 2.0 to 7.0.

Embodiment 78. The method of any one of embodiments 61-77, wherein the pH of the regeneration buffer is about pH 4.0.

Embodiment 79. The method of any one of embodiments 61-78, wherein about 3 to about 12 material volumes of the regeneration buffer are passed through the ion exchange chromatography material.

Embodiment 80. The method of any one of embodiments 61-79, wherein about 7 material volumes of the regeneration buffer are passed through the ion exchange chromatography material.

Embodiment 81. The method of any one of embodiments 61-80, wherein the acid wash is about 167M NaOAc and about 0.3M phosphoric acid.

Embodiment 82. The method of any one of embodiments 61-81, wherein about 1 to about 5 material volumes of the acid wash are passed through the ion exchange chromatography material.

Embodiment 83. The method of any one of embodiments 61-82, wherein about 3 material volumes of the acid wash are passed through the ion exchange chromatography material.

Embodiment 84. The method of any one of embodiments 61-83, wherein about 2 to about 12 material volumes of sanitization buffer are passed through the ion exchange chromatography material.

Embodiment 85. The method of any one of embodiments 61-84, wherein about 4 material volumes of sanitization buffer are passed passing a regeneration buffer through the ion exchange chromatography material.

Embodiment 86. The method of any one of embodiments 61-85, wherein the ion exchange chromatography material is stored in about 1 to 5 material volumes of storage buffer.

Embodiment 87. The method of any one of embodiments 61-86, wherein the ion exchange chromatography material is stored in about 3 material volumes of storage buffer.

Embodiment 88. A method for purifying a polypeptide from a composition comprising the polypeptide and an impurity using an ion exchange chromatography material wherein the ion exchange chromatography material is suitable for reuse, the method comprising the steps of
a) equilibrating the ion exchange chromatography material with an equilibration buffer;
b) loading the composition on the ion exchange chromatography material, wherein the load density is ≤about 1000 g/L;
c) collecting fractions comprising the polypeptide;
d) washing the ion exchange chromatography material with equilibration buffer;
e) passing a regeneration buffer through the ion exchange chromatography material, wherein the regeneration buffer comprises Tris and sodium acetate;
f) passing a sanitization buffer through the ion exchange chromatography material, wherein the sanitization buffer comprises about 0.5 N NaOH; and
g) storing the ion exchange chromatography material in a storage buffer, wherein the storage buffer comprises about 0.1 N NaOH.

Embodiment 89. A method for purifying a polypeptide from a composition comprising the polypeptide and an impurity using an ion exchange chromatography material wherein the ion exchange chromatography material is suitable for reuse, the method comprising the steps of
a) equilibrating the ion exchange chromatography material with an equilibration buffer;
b) loading the composition on the ion exchange chromatography material, wherein the load density is ≥20 g/L;
c) collecting fractions comprising the polypeptide;
d) washing the ion exchange chromatography material with equilibration buffer;
e) passing a regeneration buffer through the ion exchange chromatography material, wherein the regeneration buffer comprises Tris and sodium acetate;
f) passing a sanitization buffer through the ion exchange chromatography material, wherein the sanitization buffer comprises about 0.5 N NaOH; and
g) storing the ion exchange chromatography material in a storage buffer, wherein the storage buffer comprises about 0.1 N NaOH.

Embodiment 90. The method of embodiment 89, wherein the load density is ≥70 g/L.

Embodiment 91. A method for purifying a polypeptide from a composition comprising the polypeptide and an impurity using an ion exchange chromatography material wherein the ion exchange chromatography material is suitable for reuse, the method comprising the steps of
a) equilibrating the ion exchange chromatography material with an equilibration buffer;
b) loading the composition on the ion exchange chromatography material, wherein the load density exceeds the dynamic binding capacity of the ion exchange chromatography for the polypeptide;
c) collecting fractions comprising the polypeptide;
d) washing the ion exchange chromatography material with equilibration buffer;
c) passing a regeneration buffer through the ion exchange chromatography material, wherein the regeneration buffer comprises Tris and sodium acetate;
f) passing a sanitization buffer through the ion exchange chromatography material, wherein the sanitization buffer comprises about 0.5 N NaOH; and
g) storing the ion exchange chromatography material in a storage buffer, wherein the storage buffer comprises about 0.1 N NaOH.

Embodiment 92. The method of embodiment 91, wherein the load density is at least about 2× the dynamic binding capacity of the ion exchange chromatography material for the polypeptide.

Embodiment 93. The method of embodiment 91, wherein the load density is about 2× to about 100× the dynamic binding capacity of the ion exchange chromatography material for the polypeptide.

Embodiment 94. The method of embodiment 91, wherein the load density is about 10× the dynamic binding capacity of the ion exchange chromatography material for the polypeptide.

Embodiment 95. The method of embodiment 94, wherein the equilibration buffer comprises about 20 mM to about 60 mM sodium acetate.

Embodiment 96. The method of any one of embodiments 88-95, wherein the equilibration buffer comprises about 40 mM sodium acetate.

Embodiment 97. The method of any one of embodiments 88-96, wherein the pH of the equilibration buffer is about pH 5.0 to about pH 6.0.

Embodiment 98. The method of any one of embodiments 88-97, wherein the pH of the equilibration buffer is about pH 5.5.

Embodiment 99. The method of any one of embodiments 88-98, wherein the ion exchange chromatography material is washed with about 3-7 material volumes of equilibration buffer.

Embodiment 100. The method of any one of embodiments 88-99, wherein the ion exchange chromatography material is washed with about 5 material volumes of equilibration buffer.

Embodiment 101. The method of any one of embodiments 88-100, wherein the regeneration buffer comprises about 25 to about 75 mM Tris and about 50 to about 100 mM NaOAc.

Embodiment 102. The method of any one of embodiments 88-101, wherein the regeneration buffer comprises about 50 mM Tris and about 85 mM NaOAc.

Embodiment 103. The method of any one of embodiments 88-102, wherein the pH of the regeneration buffer is about pH 7 to 9.

Embodiment 104. The method of any one of embodiments 88-103, wherein the pH of the regeneration buffer is about pH 8.0.

Embodiment 105. The method of any one of embodiments 88-104, wherein about 3 to about 12 material volumes of regeneration buffer are passed through the ion exchange chromatography material.

Embodiment 106. The method of any one of embodiments 88-105, wherein about 10 material volumes of regeneration buffer are passed through the ion exchange chromatography material.

Embodiment 107. The method of any one of embodiments 88-106, wherein about 2 to about 12 material volumes of sanitization buffer are passed through the ion exchange chromatography material.

Embodiment 108. The method of any one of embodiments 88-107, wherein about 10 material volumes of sanitization buffer are passed passing a regeneration buffer through the ion exchange chromatography material.

Embodiment 109. The method of any one of embodiments 88-108, wherein the ion exchange chromatography material is stored in about 1 to 5 material volumes of storage buffer.

Embodiment 110. The method of any one of embodiments 88-109, wherein the ion exchange chromatography material is stored in about 4 material volumes of storage buffer.

Embodiment 111. The method of any one of embodiments 1-110, wherein the ion exchange chromatography material is in a chromatography column.

Embodiment 112. The method of any one of embodiments 1-111, wherein the ion exchange chromatography material is a cation exchange chromatography material.

Embodiment 113. The method of any one of embodiments 1-112, wherein the ion exchange chromatography material comprises a sulfopropyl moiety linked to a support matrix.

Embodiment 114. The method of embodiment 113, wherein the support matrix comprises cross-linked poly(styrenedivinylbenzene).

Embodiment 115. The method of any one of embodiments 1-114, wherein the ion exchange chromatography material is a POROS™ HS50 material.

Embodiment 116. The method of any one of embodiments 1-115, wherein the ion exchange chromatography material is a mixed mode cation exchange material.

Embodiment 117. The method of embodiment 116, wherein the mixed mode cation exchange material is Capto MMC™, Capto MMC™ ImpRes, Nuvia™ cPrime™, or Toyopearl NLX Trp-650M.

Embodiment 118. The method of any one of embodiments 1-111, wherein the ion exchange chromatography material is an anion exchange chromatography material.

Embodiment 119. The method of any one of embodiments 1-111 or 118, wherein the ion exchange chromatography material comprises a primary amine, a secondary amine, a tertiary amine or a quaternary ammonium ion functional group, a polyamine functional group, or a diethylaminoethyl functional group linked to a support matrix.

Embodiment 120. The method of any one of embodiments 1-111, 118 or 119, wherein the ion exchange chromatography material is a mixed mode anion exchange material.

Embodiment 121. The method of embodiment 120, wherein the mixed mode anion exchange material is Capto Adhere™ or Capto Adhere™ ImpRes.

Embodiment 122. The method of any one of embodiments 1-121, wherein the ion exchange chromatography material is used for large-scale production of the polypeptide.

Embodiment 123. The method of any one of embodiments 1-122, wherein the polypeptide is an antibody, an immunoadhesin, an Fc-containing protein, or an immunoconjugate.

Embodiment 124. The method of embodiment 123, wherein the polypeptide is an antibody.

Embodiment 125. The method of embodiment 124, wherein the antibody is a monoclonal antibody.

Embodiment 126. The method of embodiment 125, wherein the monoclonal antibody is a chimeric antibody, humanized antibody, or human antibody.

Embodiment 127. The method of embodiment 125 or 126, wherein the monoclonal antibody is an IgG monoclonal antibody.

Embodiment 128. The method of any one of embodiments 124-127, wherein the antibody is an antigen binding fragment.

Embodiment 129. The method of embodiment 128, wherein the antigen binding fragment is a Fab fragment, a Fab′ fragment, a F(ab′)2 fragment, a scFv, a di-scFv, a bi-scFv, a tandem (di, tri)-scFv, a Fv, a sdAb, a tri-functional antibody, a BiTE, a diabody or a triabody.

Embodiment 130. The method of any one of embodiments 124-129, wherein the antibody is a bispecific antibody.

Embodiment 131. The method of any one of embodiments 124-127, wherein the antibody is selected from the group consisting of an anti-CD20 antibody, an anti-CD40 antibody, an anti-HER2 antibody, an anti-IL6 antibody, an anti-IgE antibody, an anti-IL13 antibody, an anti-Flu A antibody, an anti-TIGIT antibody, an anti-PD-L1 antibody, an anti-VEGF-A antibody, an anti-VEGF-A/ANG2 antibody, an anti-CD79b antibody, an anti-ST2 antibody, an anti-factor D antibody, an anti-factor IX antibody, an anti-factor X antibody, an anti-abeta antibody, an anti-tau antibody, an anti-CEA antibody, an anti-CEA/CD3 antibody, an anti-CD20/CD3 antibody, an anti-FcRH5/CD3 antibody, an anti-Her2/CD3 antibody, an anti-FGFR1/KLB antibody, a FAP-4-1 BBL fusion protein, and a FAP-IL2v fusion protein.

Embodiment 132. The method of any one of embodiments 124-127, wherein the antibody is selected from the group consisting of ocrelizumab, pertuzumab, trastuzumab, tocilizumab, faricimab, polatuzumab, gantenerumab, cibisatamab, crenezumab, mosunetuzumab, tiragolumab, bevacizumab, rituximab, atezolizumab, obinutuzumab, lampalizumab, lebrikizumab, omalizumab ranibizumab, emicizumab, selicrelumab, prasinezumab, RO6874281 and RO7122290.

Embodiment 133. The method of any one of embodiments 124-132, wherein the polypeptide is purified using an affinity chromatography material prior to passing through the ion exchange chromatography material, wherein the ion exchange chromatography material is a cation exchange chromatography material.

Embodiment 134. The method of embodiment 133, wherein the affinity material is selected from the group consisting of a Protein A chromatography, a Protein G chromatography, a Protein A/G chromatography, a protein L chromatography, a FcXL chromatography, a protein XL chromatography, a kappa chromatography, and a kappaXL chromatography.

Embodiment 135. The method of embodiment 134, wherein the Protein A affinity material is a MAbSelect material, a MAbSelect SuRe™ material or a MAbSelect SuRe™ LX material.

Embodiment 136. The method of any one of embodiments 1-135, wherein the buffers are passed through the ion exchange chromatography material at about 15-20 material volumes/hour.

Embodiment 137. The method of any one of embodiments 1-136, wherein the regeneration buffer is passed through the ion exchange chromatography material at about 10 material volumes/hour.

Embodiment 138. The method of any one of embodiments 1-137, wherein the polypeptide is produced in a host cell.

Embodiment 139. The method of embodiment 138, wherein the host cell is a Chinese hamster ovary (CHO) cell.

Embodiment 140. The method of any one of embodiments 1-139, wherein the impurity comprises one or more of IgG fragments, host cell proteins, or host cell nucleic acids.

Embodiment 141. The method of embodiment 140, wherein the polypeptide is produced in a CHO cell and the impurity comprises CHO host cell protein (CHOP) and/or CHO nucleic acids.

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

Further details of the invention are illustrated by the following non-limiting Examples. The disclosures of all references in the specification are expressly incorporated herein by reference.

### EXAMPLES

The examples below are intended to be purely exemplary of the invention and should therefore not be considered to limit the invention in any way. The following examples and detailed description are offered by way of illustration and not by way of limitation.

### Example 1. Development of a Cation Exchange Overload Regeneration Wash Step

A regeneration step was developed to reduce fouling during sanitization of an overload cation exchange chromatography material and to minimize impurity carry-over between chromatography runs.

### Materials and Methods

### Feedstock

The present studies were performed using adjusted antibody MabSelect SuRe™ (MSS) pools derived from 400 L Pilot Plant runs, using CHO cell lines. The 400 L cell culture production was harvested via continuous (hermetic) centrifugation, followed by depth filtration, and final sterilizing-grade (0.2 μm) filtration.

### Buffers

The buffers listed in Table 2 were used in the present POROS™ 50HS (OL) chromatography processes.

TABLE 2
POROS ™ 50HS (OL) chromatography buffers

| | | Conductivity |
|---|---|---|
| Buffer | pH | (mS/cm) |
| Equilibration/Wash | 5.5 ± 0.10 | 2.7 ± 0.3 |
| 40 mM sodium acetate | | |
| CEX OL Regeneration Wash | 8.00 ± 0.10 | 8.6 ± 0.7 |
| 50 mM Tris, 85 mM sodium acetate | | |
| CEX OL Regeneration Wash | 8.30 ± 0.10 | 20 ± 2 |
| 350 mM sodium acetate | | |
| Sanitization | N/A | 55 ± 5 after |
| 0.5M NaOH | | 1:1 dilution |
| | | with water |
| Storage | N/A | 22 ± 5 |
| 0.1N NaOH | | |

### Equipment

All lab-scale chromatography development work was performed using GE Healthcare AKTA™ Avant 150 and Omnifit® 0.66 cm diameter columns.

### Purification Methods

The methods tabulated below methods for POROS™ 50HS (overload mode) and Capto™ Adhere steps. Scale-independent parameters have been provided wherever possible.

### Cation Exchange Overload (OL) Chromatography (POROS™ 50 HS)

POROS™ 50HS chromatography was operated in overload mode where the feedstock is loaded at pH 5.5, followed by a post-load wash with the same equilibration buffer. Since the overload mode requires a high load density to offset yield loss, a higher pH and conductivity regeneration step is required with sodium acetate to wash off any bound protein impurities, prior to the sanitization and storage with sodium hydroxide. When utilizing die overload mode, the POROS™ 50HS resin was loaded at an optimal binding capacity that exceeds the dynamic binding capacity of the mAb. This results in protein-related impurities (e.g. vHMWS and HMWS) and process-related impurities (e.g. CHOP) to competitively bind to the resin, while causing the mAb to breakthrough; thus, the breakthrough is then collected as the overload pool. Increasing load density results in an increase in recovered yield.

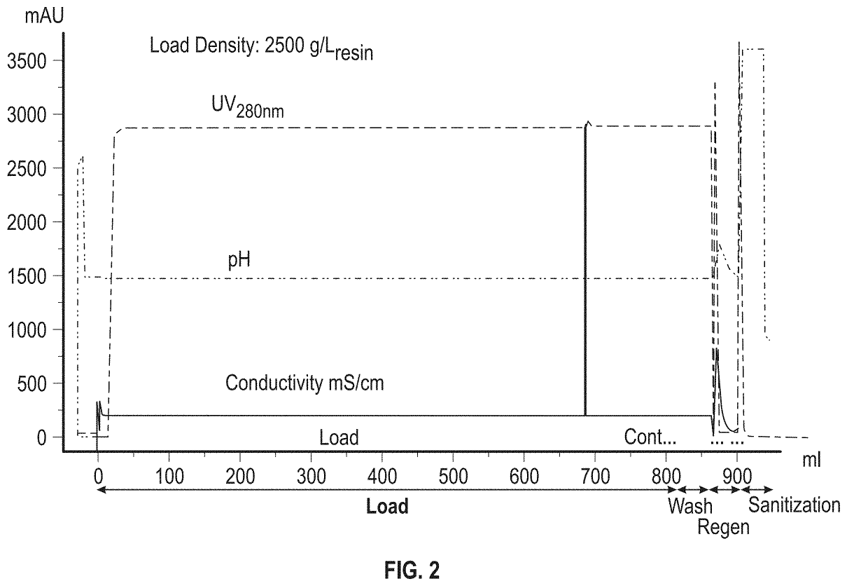

The POROS™ 50HS chromatography target parameters, phase information and running conditions are described in Table 3. FIG. 2 shows a representative Poros Overload chromatogram.

TABLE 3
POROS ™ 50-HS Chromatography Process,
Phase Information and Running Conditions

| | |
|---|---|
| Resin | POROS ™ 50HS |
| Mode | Overload |
| Load Density | ≤1000 g/L |
| Load | Adjusted MabSelect SuRe ™ pool, pH 5.5 |
| Flow rate | 15-20 CV/hr (10 CV/hr for regeneration phase) |
| Bed Height | 20 cm |
| Pooling Criteria | 0.5-0.5 OD |

| Step | Buffer | CV |
|---|---|---|
| Equilibration | 40 mM sodium acetate, pH | 5 |
| | 5.5 | |
| Load | Adjusted MabSelect SuRe ™ | — |
| | Pool, pH 5.5 | |
| Wash | 40 mM sodium acetate, pH | 5 |
| | 5.5 | |
| Regeneration | Evaluated: | 10 |
| Buffers | 1.) 50 mM Tris, 85 mM | |
| | sodium acetate, pH 8.0 | |
| | 2.) 350 mM sodium acetate, | |
| | pH 8.3 | |
| Sanitization | 0.5N NaOH | 10 |
| Storage | 0.1N NaOH | 4 |

### Analytical Methods

### Product Protein Concentration

MAb concentrations were determined by using an Agilent 8453 spectrophotometer with a quartz cuvette at 280 nm and 320 nm. Samples are diluted with purified water (PW) to reach an absorbance between 0.1 to 1.0 AU at 280 nm. The following equation was used to calculate protein concentration:

$$Protein Concentration = ( A 280 - A 320 ) \text{/} ( extinction {coefficient}^{*} path length ) \times Dilution Factor$$

$$Total protein ( mg ) = protein concentration ( \frac{mg}{ml} ) \times pool volume ( ml )$$

### Size Exclusion Chromatography (SEC) for Size Variants

The relative ratio of Very High Molecular Weight Species (vHMWS), High Molecular Weight Species (HMWS), Total Aggregates (vHMWS+HMWS), monomer, and fragments was determined by size-exclusion chromatography (SEC-HPLC) using TSK G3000SWXL (7.8×300 mm) analytical HPLC columns (Tosoh Bioscience). The running conditions are listed below in Table 4. The column was run at 0.5 ml/min for 30 minutes in the running buffer listed in the table, and monitored at 280 nm. The auto-sample chamber is set to 5° C. with a range of 2-8° C.; however, the column compartment is set to ambient temperature. Samples were injected neat with a target mass load of 25-50 μg. The HPLC system used for this assay is Agilent 1100/1200.

TABLE 4
SEC running conditions

| | |
|---|---|
| Column | TSK G3000SWXL (7.8 × 300 mm) |
| Vendor | Tosoh Bioscience (part # 08541, VWR |
| | catalog # 100368-358) |
| Flow rate | 0.5 mL/min |
| Temperature | ambient |
| Run time | 30 minutes |
| Run mode | Isocratic |
| Mobile phase | 0.20M potassium phosphate, 0.25M potassium |
| | chloride, pH, 6.2 ± 0.1 |
| UV detection | 280 nm |
| Target injection | 25-50 μg |

### Analytical Operations Assays

The following assays were performed during process development and run analysis: CHO DNA by Taqman PCR

Carryover by Capillary Zone Electrophoresis/Laser-Induced Fluorescence (CZE-LIF) Endotoxin by kinetic assay for the detection of Gram-negative bacterial endotoxin.

### Regeneration Wash Step Development

mAb1 POROS™ 50HS (OL) Column Regeneration Step and Carry-Over Study

The CEX overload chromatography operation includes a regeneration step to effectively remove any bound impurities, specifically aggregates, from the POROS™ 50 HS resin. Two regeneration buffers were evaluated to effectively remove the impurities: 350 mM sodium acetate, pH 8.3; and 50 mM Tris, 85 mM sodium acetate, pH 8.0.

During the regeneration buffer evaluation, two CEX overload runs were loaded up to 1000 g/Lᵣₑₛᵢₙ and incorporated two regeneration phases (5 CV duration of each buffer); however, with each run, the sequence of regeneration buffers was varied. Below are the two evaluation runs with the sequence of regeneration buffers tested:

### Evaluation Run A:

Regeneration Phase 1: 50 mM Tris, 85 mM NaOAc, pH 8.0

Regeneration Phase 2: 350 mM NaOAc, pH 8.3

### Evaluation Run B:

Regeneration Phase 1: 350 mM NaOAc, pH 8.3

Regeneration Phase 2: 50 mM Tris, 85 mM NaOAc, pH 8.0

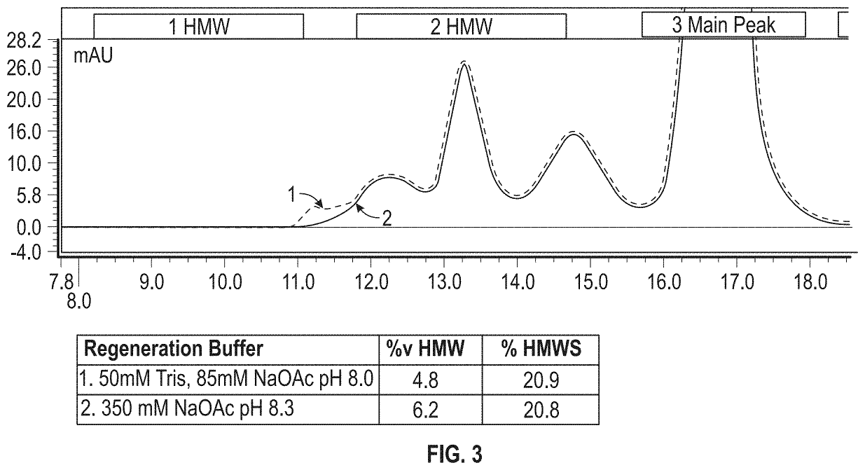

The typical outputs were analyzed (KPI, impurity clearance and product quality); and both runs obtained similar performance. However, in addition to those outputs, the column effluent during the first regeneration phase was collected and analyzed on the SEC for assessment of aggregate removal from the column, which is summarized by FIG. 3 (an overlay of the SEC chromatograms of the first regeneration phase column effluent). FIG. 3 shows that the phase using the 350 mM NaOAc pH 8.3 buffer has a higher level of vHMWS, indicating that it had removed more aggregates from the column, and is therefore a better regeneration wash buffer. No column fouling was observed using either regeneration buffer.

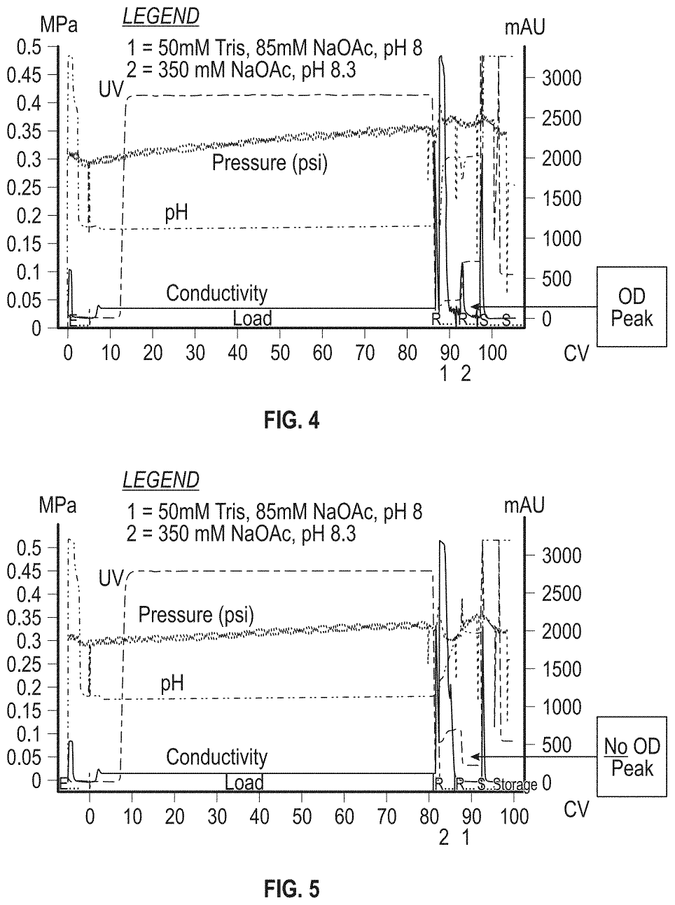

When the evaluation run chromatograms are analyzed (FIG. 4, the chromatogram of Evaluation Run A, and FIG. 5, the chromatogram of Evaluation Run B), there is no OD peak observed in the regeneration phase 2 after the 350 mM NaOAc pH 8.3 regeneration buffer is used, indicating that there was no residual protein in the column after it was used. No pressure spikes were observed during the process using either regeneration buffer. No residual protein carryover was observed by analytical observation assays (Table 5).

TABLE 5
Protein Carry Over Results

| | Total Protein | CHO DNA | Endotoxin |
|---|---|---|---|
| Carryover Data | (μg/ml) | (pg/ml) | (EU/ml) |
| Labscale Column | <0.25 | <1 | Not Tested |
| (after 11 runs) | | | |
| Pilot Plant Column | <0.25 | <1 | <0.1 |
| (after 2 runs) | | | |

mAb2 POROS™ 50HS (OL) Column Regeneration Step and Carry-Over Study

The CEX column regeneration step was optimized to effectively remove impurities from the Poros™ 50HS resin and prevent run-to-run carry-over and resin fouling during the sanitization phase. Two regeneration buffers were tested: 50 mM Tris, 85 mM sodium acetate, pH 8.0 and 350 mM sodium acetate, pH 8.3. During the regeneration buffer evaluation, two CEX overload runs were loaded up to 800 g/Lᵣₑₛᵢₙ and incorporated two regeneration phases (5 CV duration of each buffer), however, with each run, the sequence of regeneration buffers is varied. Below is the two evaluation runs with the sequence of regeneration buffers tested:

### Evaluation Run A:

Regeneration Phase 1: 50 mM Tris, 85 mM NaOAc, pH 8.0
Regeneration Phase 2: 350 mM NaOAc, pH 8.3

### Evaluation Run B:

Regeneration Phase 1: 350 mM NaOAc, pH 8.3
Regeneration Phase 2: 50 mM Tris, 85 mM NaOAc, pH 8.0

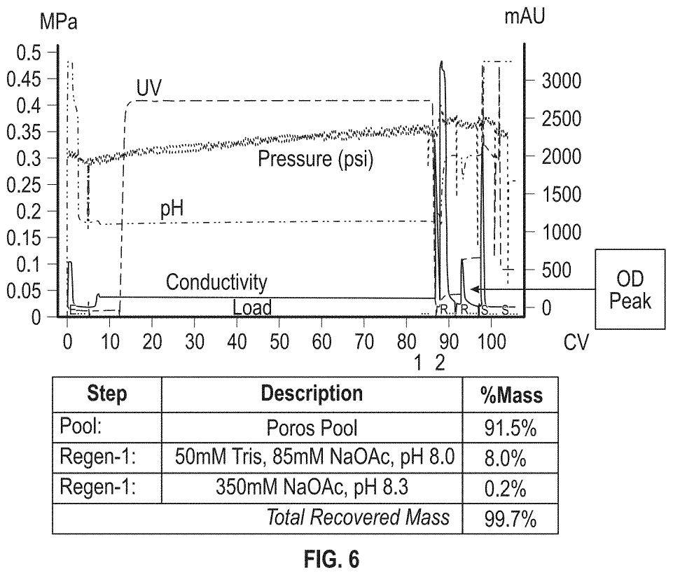

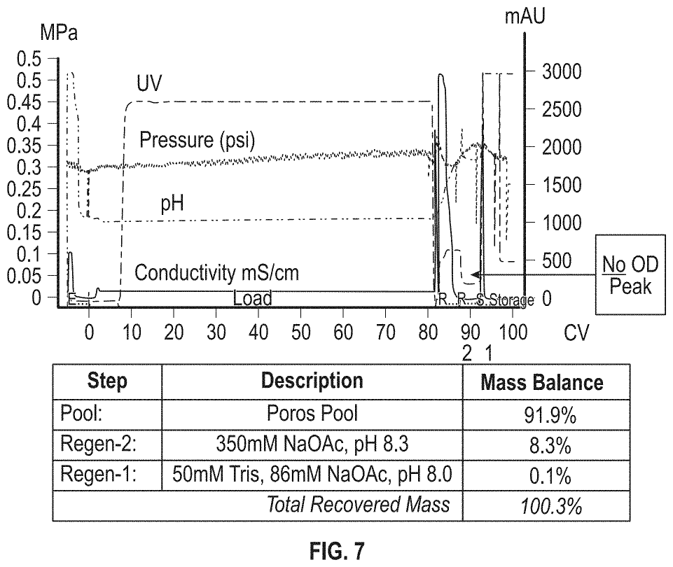

When the evaluation run chromatograms are analyzed (FIG. 6, the chromatogram of Evaluation Run A, and FIG. 7, the chromatogram of Evaluation Run B), there is no OD peak observed in the regeneration phase 2 after the 350 mM NaOAc pH 8.3 regeneration buffer is used, indicating that there is no residual protein in the column after it was used. No pressure spikes were observed during the process using either regeneration buffer.

To verify the cleaning procedure, one product contacting run was performed using each regeneration buffer. A mock run (a run performed without the load phase) was performed after the product contacting run using the procedure detailed in Table 6. For the mock runs, the two regeneration buffers were alternated from previous protein contacting run to assess protein carryover. Below is a summary of the run sequence for the evaluation:

1.) Protein contacting run using 50 mM Tris, 85 mM NaOAc, pH 8.0 regeneration phase

2.) Mock run using 350 mM NaOAc, pH 8.3 regeneration phase

3.) Protein contacting run using 350 mM NaOAc, pH 8.3 regeneration phase

4.) Mock run using 50 mM Tris, 85 mM NaOAc, pH 8.0 regeneration phase

TABLE 6
Poros ™ 50HS Mock-Run Cleaning Procedure

| | Phase | |
|---|---|---|
| Step | Duration (CV) | Buffer |
| Equilibration | 5 | 40 mM NaOAc, pH 5.5 |
| Elution (mock pool)* | 4 | 500 mM NaOAc, pH 5.5 |
| Equilibration | 4 | 40 mM NaOAc, pH 5.5 |
| Regeneration | 5 | 350 mM NaOAc, pH 8.3 |
| Sanitization | 4 | 0.5N NaOH |
| Storage | 3 | 0.1N NaOH |

*Mock pools for the two regeneration buffers tested were collected and submitted for Y:99 (total protein) and Y:77 (CHO DNA) analysis.

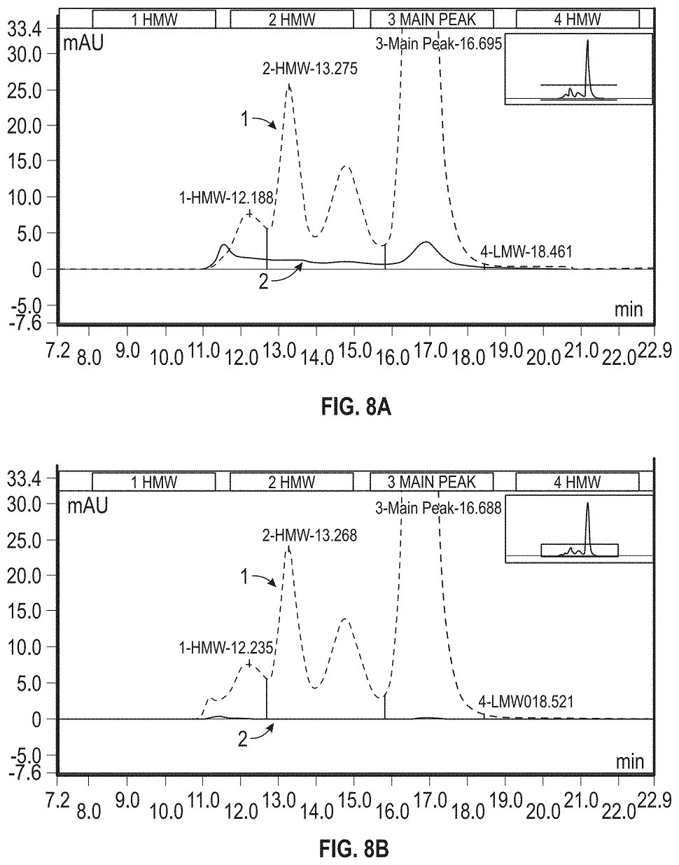

The regeneration phases from both the protein contacting runs and the mock runs were collected and run on SEC (FIGS. 8A and 8B). Mock pools were collected and analyzed for CHO DNA (Y:77) and total protein (Y:99).

Results listed in Table 7 showed comparable performance for both regeneration buffers; CHO DNA and total protein were reduced to ≤LOQ following the product contacting run on column. SEC analysis of regeneration solutions for protein contacting and mock runs showed that 350 mM NaOAc, pH 8.3 was able to elute bound protein more effectively than the 50 mM Tris, 85 mM NaOAc, pH 8.0 during the protein contacting run. When using 50 mM Tris, 85 mM NaOAc, pH 8.0, the protein contacting run still left protein bound to the column, as indicated by the additional protein eluting when using 350 mM NaOAc, pH 8.3 in the subsequent mock run (FIGS. 8A and 8B).

TABLE 7
Poros ™ 50HS Mock-Run ‘Carry-Over’ Cleaning Results

| | CHO DNA, | Total Protein, |
|---|---|---|
| Buffer | Y:77 (pg/mL) | Y:99* (μg/mL) |
| 50 mM Tris, 85 mM | ≤1 | ≤0.25 |
| NaOAc, pH 8.0 | | |
| 350 mM NaOAc, pH 8.3 | ≤1 | ≤0.25 |

*Carry-over (Y99) samples were treated with 0.1% PS20 within 30-min of sample collection and stored at ≤−70° C., prior to analysis.

### Conclusions

The 350 mM NaOAc pH 8.3 and 50 mM Tris, 85 mM NaOAc, pH 8.0 regeneration effectively mitigated Poros™ 50HS resin fouling for mAb2 and mAb1. Minimal pressure increase was observed during the sanitization phase using either buffer. The 350 mM NaOAc regeneration buffer was selected over the 50 mM Tris NaOAc pH 8.0 buffer as a platform regeneration buffer for CEX overload chromatography.

### Example 2 Development of a Multimodal Anion Exchange Overload Regeneration Wash Step

A regeneration step was developed to reduce fouling during sanitization of an overload multi-modal anion exchange chromatography material and to minimize impurity carry-over between chromatography runs.

### Materials and Methods

### Feedstock

The present studies were performed using adjusted antibody MabSelect SuRe™ (MSS) pools derived from 400 L Pilot Plant runs, using CHO cell lines. The 400 L cell culture production was harvested via continuous (hermetic) centrifugation, followed by depth filtration, and final sterilizing-grade (0.2 μm) filtration.

### Overload (OL) Chromatography (Capto™ Adhere)

During the “overload” operation, the column is loaded under conditions in which both product and impurities exhibit strong binding (log Kₚ>2.0), through the point at which protein breakthrough begins to occur. Optimal performance may be realized when the impurities bind more strongly than the product protein (i.e. impurity log Kₚ>mAb log Kₚ), such that the product protein will experience displacement by impurity. This results in an eluent, after breakthrough, which is product-enriched and impurity-depleted.

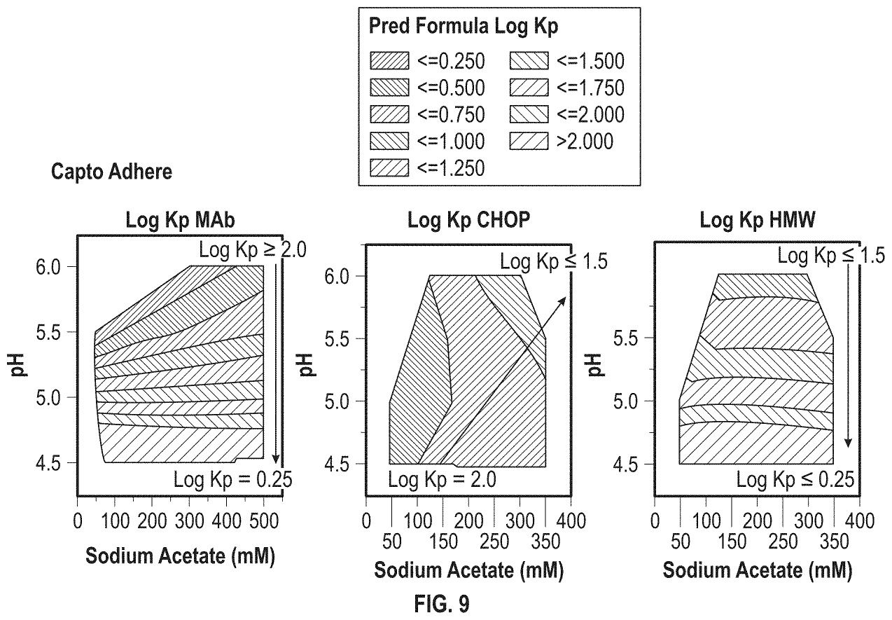

For Capto™ Adhere overload chromatography study, the feedstock is loaded at pH 8.0, a high log Kₚ (binding condition) as shown in FIG. 9. The load phase is followed by a post-load wash with equilibration buffer. Process conditions are described in Tables 8 and 9. The regeneration buffers tested are listed in Table 10.

All lab-scale chromatography development work was performed using GE Healthcare AKTA Avant 150 and Omnifit 0.66 cm diameter columns.

TABLE 8
Capto ™ Adhere Chromatography Process, Phase
Information and Running Conditions

| | |
|---|---|
| Resin | Capto ™ Adhere |
| Mode | Overload |
| Load Density | 300 g/L |
| Load | Adjusted MabSelect SuRe pool, pH 8.0 |
| Flow rate | 150 cm/hr |
| Bed Height | 20 cm |
| Pooling Criteria | 0.5-0.5 OD |

TABLE 9

| | | Column |
|---|---|---|
| Step/Phase | Buffer | Volume (CV) |
| Equilibration | 50 mM Tris, 85 mM sodium | 5 |
| | acetate, pH 8.0 | |
| Load | Adjusted MabSelect SuRe | As Required |
| | Pool, pH 8.0 | |
| Wash | 50 mM Tris, 85 mM sodium | 10 |
| | acetate, pH 8.0 | |
| Regeneration | Buffer's Evaluated: | 7 |
| Wash 1 | 1.) 350 mM sodium | |
| | acetate, pH 8.3 | |
| | 2.) 40 mM NaOAc pH 4.0 | |
| | 3.) 40 mM NaOAc pH 5.0 | |
| | 4.) 350 mM NaOAc pH 4.0 | |
| | 5.) 350 mM NaOAc pH 5.0 | |
| Regeneration | See buffer list above and | 5 |
| Wash 2 | Table 3.1-1 for details. | |
| Acid Wash | 0.167M Acetic Acid, 0.3M | 3 |
| | Phosphoric Acid | |
| Sanitization | 0.5N NaOH, 1M NaCl | 4 |
| Storage | 0.1N NaOH | 3 |

TABLE 10
Capto ™ Adhere Regeneration Wash Buffers

| | | Conductivity |
|---|---|---|
| Regeneration Wash Buffer | pH | (mS/cm) |
| 350 mM sodium acetate | 8.3 ± 0.1 | 20.0 ± 3 |
| 40 mM sodium acetate | 4.0 ± 0.1 | 1.0 ± 1.0 |
| 40 mM sodium acetate | 5.0 ± 0.1 | 2.6 ± 1.0 |
| 350 mM sodium acetate | 4.0 ± 0.1 | 6.6 ± 5 |
| 350 mM sodium acetate | 5.0 ± 0.1 | 18.0 ± 5 |

### Analytical Methods

### Product Protein Concentration

MAb concentrations were determined by using SoloVPE at 278 nm. The following equation was used to calculate protein concentration:

$$Protein Concentration = ( A 280 - A 320 ) \text{/} ( extinction {coefficient}^{*} path length ) \times Dilution Factor$$

$$Total protein ( mg ) = protein concentration ( \frac{mg}{ml} ) \times pool volume ( ml )$$

### Regeneration Wash Step Development

MAB3 Capto™ Adhere (OL) Column Regeneration Study Overview

The overload chromatography operation includes a regeneration step to effectively remove any bound impurities. Regeneration buffers evaluated are described in Table 10. For the regeneration buffer evaluation, the column was loaded to 300 g/Lresin and incorporated two regeneration phases. With each run, the sequence of regeneration buffers was varied. The sequence of regeneration buffers tested is summarized in Table 11 and detailed below.

TABLE 11
Sequence of buffer used for the
regeneration wash phase. The number indicates
the order in which the regeneration buffer was used.

| Regeneration Buffer | Run A | Run B | Run C | Run D | Run E |
|---|---|---|---|---|---|
| 350 mM NaOAc, pH 8.3 | 1 | | | | |
| mS/cm* | | | | | |
| 40 mM NaOAc, pH 4.0 | 2 | 1 | | 2 | |
| 350 mM NaOAc, pH 4.0 | | 2 | | 1 | |
| 40 mM NaOAc, pH 5.0 | | | 1 | | 2 |
| 350 mM NaOAc, pH 5 0 | | | 2 | | 1 |

*5CV phase duration was used instead of 7CV

### Evaluation Run A:

Regeneration Phase 1: 350 mM sodium acetate, pH 8.3
Regeneration Phase 2: 40 mM sodium acetate, pH 4.0

### Evaluation Run B:

Regeneration Phase 1: 40 mM sodium acetate, pH 4.0
Regeneration Phase 2: 350 mM sodium acetate, pH 4.0

### Evaluation Run C:

Regeneration Phase 1: 40 mM sodium acetate, pH 5.0
Regeneration Phase 2: 350 mM sodium acetate, pH 5.0

### Evaluation Run D:

Regeneration Phase 1: 350 mM sodium acetate, pH 4.0
Regeneration Phase 2: 40 mM sodium acetate, pH 4.0

### Evaluation Run E:

Regeneration Phase 1: 350 mM sodium acetate, pH 5.0
Regeneration Phase 2: 40 mM sodium acetate, pH 5.0

### Results

The mass balance (% protein recovery) across the phases and pressure profiles were compared to determine the effectiveness of the regeneration buffers. A higher % recovery in regeneration buffer phase indicates a more effective regeneration buffer. Table 12 shows a summary of the mass balance across phases for each run. Key experiment comparisons are summarized below.

TABLE 12
Mass Balance Summary (% Protein recovery)

| | | | | | | Total |
|---|---|---|---|---|---|---|
| | Mass Balance (% Protein Recovery) | | | | | Mass |
| | | Regener- | Regener- | | | Balance |
| | | ation | ation | Acid | Sanitization | % Protein |
| Run | Pool | Wash 1 | Wash 2 | Wash | Wash | Recovery |
| A | 75.9 | 6.7 | 21.3 | 0.2 | 0.2 | 104.3 |
| B | 72.3 | 28.1 | 0.2 | 0.1 | 0.2 | 100.9 |
| C | 72.3 | 27.2 | 0.4 | 0.7 | 0.2 | 100.8 |
| D | 72.6 | 27.7 | 0.0 | 0.1 | 0.1 | 100.5 |
| E | 72.1 | 26.6 | 0.6 | 0.6 | 0.1 | 100.0 |

### Evaluation Run A:

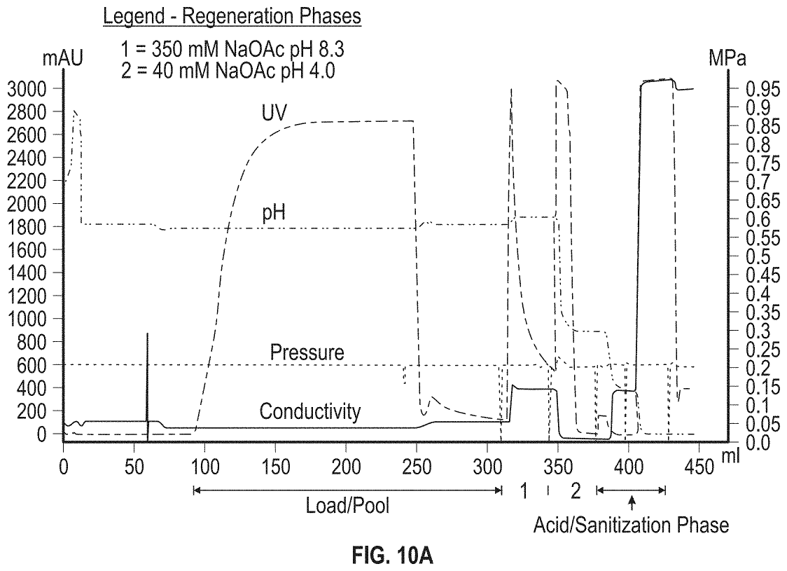

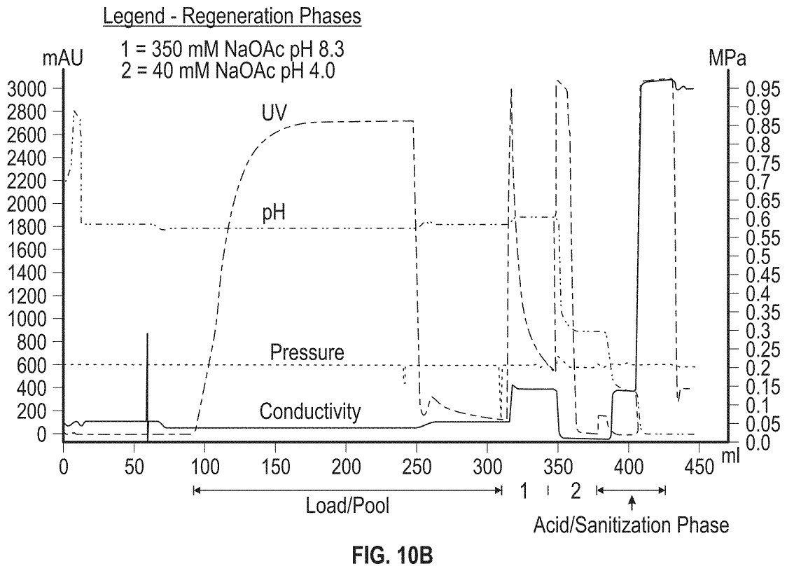

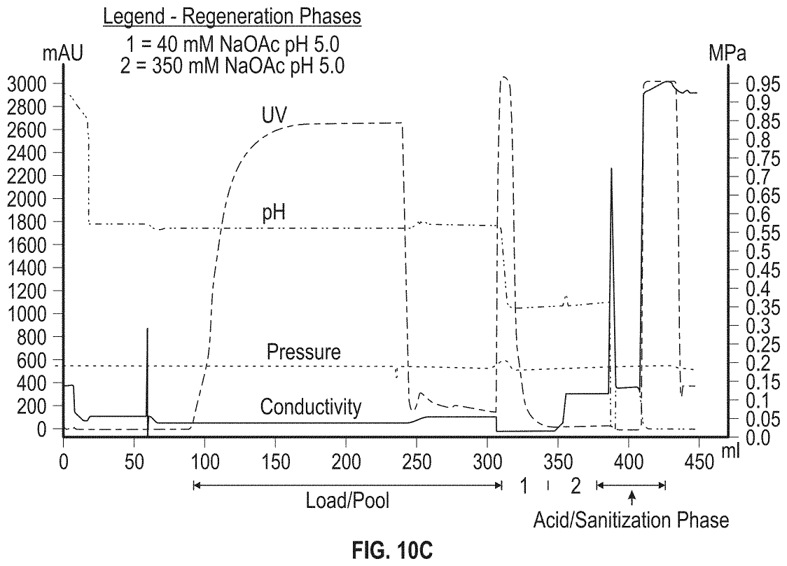

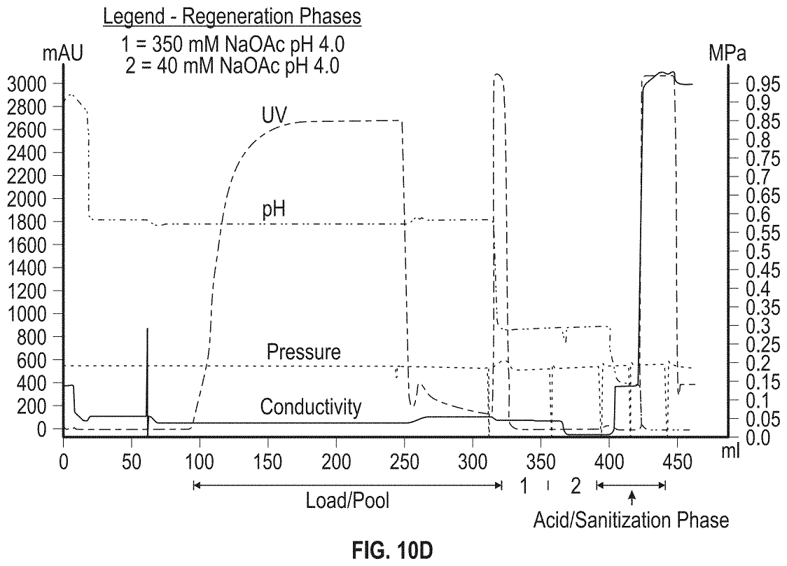

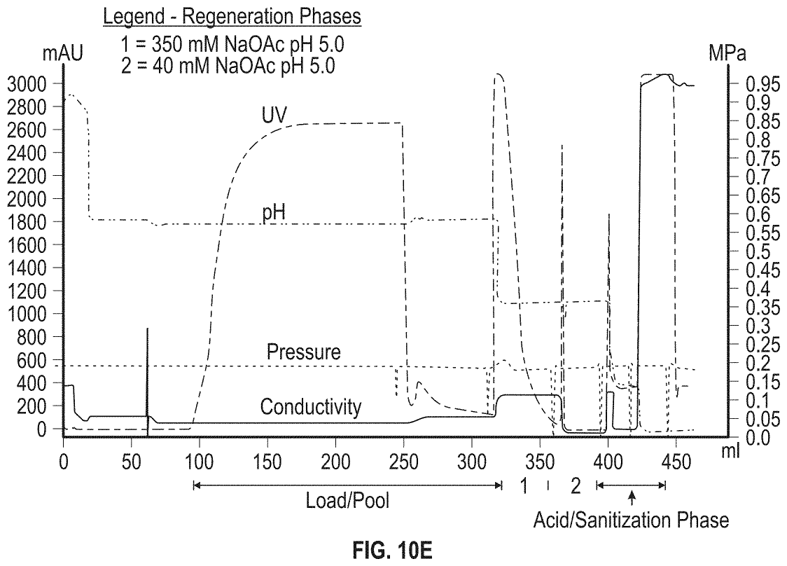

The 350 mM NaOAc pH 8.3 buffer the least effective regeneration wash. The a small amount of protein was eluted while the subsequent regeneration buffer (40 mM NaOAc, pH 4.0) was more effective as shown with the higher % recovery in the phase. Minimal protein remains in the column after the second regeneration phase. No column fouling was observed (no pressure spikes) as shown in FIG. 10A.

### Evaluation Run B Vs Run D: 40 mM Vs 350 mM NaOAc pH 4.0

The 40 mM and 350 mM NaOAc pH 4.0 buffers had equivalent effectiveness. Both regeneration wash buffers had similar protein recoveries used as the first regeneration wash phase. Minimal protein remains m the column after the first regeneration phase. No column fouling was observed (no pressure spikes) as shown in FIG. 10B and FIG. 10D.

### Evaluation Run C Vs Run E: 40 mM NaOAc Vs 350 mM NaOAc pH 5.0

The 40 mM NaOAc pH 5.0 buffers had improved effectiveness in eluting protein off the column compared to 350 mM NaOAc pH 5.0 More residual protein is elutes during regeneration wash 2 when 350 mM NaOAc pH 5.0 is used as regeneration wash 1 (Run E). An increased UV signal in regeneration wash 2 is observed (FIGS. 4,5). No column fouling was observed (no pressure spikes) as shown in FIG. 10C and FIG. 10E.

### Evaluation Run B Vs Run C: pH 4.0 Vs pH 5.0

Both pH 4.0 and 5.0 elute remaining bound protein after the pooling phase well; however, the pH 4.0 regeneration buffers are more effective than pH 5.0 at eluting bound protein. There is less residual protein elution in the post-regeneration phase 2 with the pH 4.0 conditions as shown comparing chromatograms (FIGS. 10B-10E).

### CONCLUSIONS

Lower pH is more effective as a Capto™ Adhere regeneration buffer. The 40 mM and 350 mM pH 4.0 and 5.0 are more effective Capto Adhere regeneration wash buffers compared to 350 mM NaOAc pH 8.3. The 40 mM and 350 mM NaOAc pH 4.0 regeneration buffers were the most effective regeneration buffers in this evaluation. This aligns with the binding isotherm data showing decreased protein binding to the resin at lower pH and increased binding (i.e. decreased protein elution effectiveness) at higher pH for mAB3 (FIG. 9). Minimal pressure increase was observed during the sanitization phase. There is potential to expand the number of regeneration wash buffers to elute protein off the column prior to the sanitization phase for future regeneration buffer studies.

1. A method to clean an ion exchange chromatography material for reuse wherein the chromatography material is used in overload mode in the purification of a polypeptide, the method comprising the steps of
a) passing a regeneration buffer through the ion exchange chromatography material, wherein the regeneration buffer comprises sodium acetate (NaOAc);
b) passing of sanitization buffer through the ion exchange chromatography material, wherein the sanitization buffer comprises about 0.5 N NaOH; and
c) storing the ion exchange chromatography material in a storage buffer, wherein the storage buffer comprises about 0.1 N NaOH.

2. The method of claim 1, wherein the regeneration buffer comprises about 50 to about 600 mM NaOAc, or about 350 mM NaOAc.

3. (canceled)

4. The method of claim 1, wherein the pH of the regeneration buffer is about pH 4.0 to 11, or about pH 8.3.

5. (canceled)

6. The method of claim 1, wherein about 3 to about 12 material volumes or about 10 material volumes of regeneration buffer are passed through the ion exchange chromatography material.

7. (canceled)

8. The method of claim 1, wherein about 2 to about 12 material volumes or about 10 material volumes of sanitization buffer are passed through the ion exchange chromatography material.

9. (canceled)

10. The method of claim 1, wherein the ion exchange chromatography material is stored in about 1 to 5 material volumes or about 4 material volumes of storage buffer.

11. (canceled)

12. The method of claim 1, comprising the additional step of passing an acid wash through the ion exchange chromatography material in between steps a) and b),
and wherein the sanitization buffer comprises about 0.5 N NaOH and about 1 M NaCl.

13-19. (canceled)

20. The method of claim 12, wherein the acid wash is about 167M NaOAc and about 0.3M phosphoric acid.

21. The method of claim 12, wherein about 1 to about 5 material volumes of the acid wash are passed through the ion exchange chromatography material or about 3 material volumes of the acid wash are passed through the ion exchange chromatography material.

22-26. (canceled)

27. The method of claim 1, wherein the regeneration buffer comprises Tris and sodium acetate.

28. The method of claim 27, wherein the regeneration buffer comprises about 25 to about 75 mM Tris and about 50 to about 100 mM NaOAc or about 50 mM Tris and about 85 mM NaOAc.

29-37. (canceled)

38. A method for purifying a polypeptide from a composition comprising the polypeptide and an impurity using an ion exchange chromatography material wherein the ion exchange chromatography material is suitable for reuse, the method comprising the steps of
a) equilibrating an ion exchange chromatography material with an equilibration buffer;
b) loading the composition on the ion exchange chromatography material, wherein
(i) the load density is ≤about 1000 g/L;
(ii) the load density is ≥20 g/L; or
(iii) the load density exceeds the dynamic binding capacity of the ion exchange chromatography for the polypeptide;
c) collecting fractions comprising the polypeptide;
d) washing the ion exchange chromatography material with equilibration buffer;
e) passing a regeneration buffer through the ion exchange chromatography material, wherein the regeneration buffer comprises sodium acetate;
f) passing of sanitization buffer through the ion exchange chromatography material, wherein the sanitization buffer comprises about 0.5 N NaOH; and
g) storing the ion exchange chromatography material in a storage buffer, wherein the storage buffer comprises about 0.1 N NaOH.

39. The method of claim 38, wherein the load density is ≥20 g/L.

40. (canceled)

41. The method of claim 38, wherein the load density exceeds the dynamic binding capacity of the ion exchange chromatography for the polypeptide.

42-60. (canceled)

61. The method of claim 41, further comprising the step of
passing an acid wash through the ion exchange chromatography material in between steps e) and f),
and wherein the sanitization buffer comprises about 0.5 N NaOH and about 1 M NaCl.

62-87. (canceled)

88. A method for purifying a polypeptide from a composition comprising the polypeptide and an impurity using an ion exchange chromatography material wherein the ion exchange chromatography material is suitable for reuse, the method comprising the steps of
a) equilibrating the ion exchange chromatography material with an equilibration buffer;
b) loading the composition on the ion exchange chromatography material, wherein
(i) the load density is ≤about 1000 g/L;
(ii) the load density is >20 g/L; or
(iii) the load density exceeds the dynamic binding capacity of the ion exchange chromatography for the polypeptide;
c) collecting fractions comprising the polypeptide;
d) washing the ion exchange chromatography material with equilibration buffer;
e) passing a regeneration buffer through the ion exchange chromatography material, wherein the regeneration buffer comprises Tris and sodium acetate;
f) passing a sanitization buffer through the ion exchange chromatography material, wherein the sanitization buffer comprises about 0.5 N NaOH; and
g) storing the ion exchange chromatography material in a storage buffer, wherein the storage buffer comprises about 0.1 N NaOH.

89. The method of claim 88, wherein the load density is ≥20 g/L.

90. (canceled)

91. The method of claim 88, wherein the load density exceeds the dynamic binding capacity of the ion exchange chromatography for the polypeptide.

92-110. (canceled)

111. The method of claim 1, wherein the ion exchange chromatography material is in a chromatography column.

112. The method of claim 1, wherein the ion exchange chromatography material is a cation exchange chromatography material.

113. The method of claim 1, wherein the ion exchange chromatography material comprises a sulfopropyl moiety linked to a support matrix.

114. The method of claim 113, wherein the support matrix comprises cross-linked poly(styrenedivinylbenzene).

115. (canceled)

116. The method of claim 1, wherein the ion exchange chromatography material is a mixed mode cation exchange material.

117. (canceled)

118. The method of claim 1, wherein the ion exchange chromatography material is an anion exchange chromatography material.

119. The method of claim 1, wherein the ion exchange chromatography material comprises a primary amine, a secondary amine, a tertiary amine or a quaternary ammonium ion functional group, a polyamine functional group, or a diethylaminoethyl functional group linked to a support matrix.

120. The method of claim 1, wherein the ion exchange chromatography material is a mixed mode anion exchange material.

121. (canceled)

122. The method of claim 1, wherein the ion exchange chromatography material is used for large-scale production of the polypeptide.

123. The method of claim 1, wherein the polypeptide is an antibody, an immunoadhesin, an Fc-containing protein, or an immunoconjugate.

124. The method of claim 123, wherein;
a) the polypeptide is an antibody,
b) the polypeptide is a monoclonal antibody,
c) the polypeptide is a chimeric antibody, humanized antibody, or human antibody,
d) the polypeptide is an IgG monoclonal antibody,
e) the polypeptide is an antibody wherein the antibody is an antigen binding fragment,
f) the polypeptide is an antibody wherein the antibody is an antigen binding fragment selected from a Fab fragment, a Fab′ fragment, a F(ab′)₂ fragment, a scFv, a di-scFv, a bi-scFv, a tandem (di, tri)-scFv, a Fv, a sdAb, a tri-functional antibody, a BiTE, a diabody and a triabody, or
g) the polypeptide is bispecific antibody.

125-132. (canceled)

133. The method of claim 124, wherein the polypeptide is purified using an affinity chromatography material prior to passing through the ion exchange chromatography material, wherein the ion exchange chromatography material is a cation exchange chromatography material.

134. The method of claim 133, wherein the affinity material is selected from the group consisting of a Protein A chromatography, a Protein G chromatography, a Protein A/G chromatography, a protein L chromatography, a FcXL chromatography, a protein XL chromatography, a kappa chromatography, and a kappaXL chromatography.

135. (canceled)

136. The method of claim 1, wherein the buffers are passed through the ion exchange chromatography material at about 15-20 material volumes/hour or at about 10 material volumes/hour.

137. (canceled)

138. The method of claim 1, wherein the polypeptide is produced in a host cell.

139. The method of claim 138, wherein the host cell is a Chinese hamster ovary (CHO) cell.

140. The method of claim 1, wherein the impurity comprises one or more of IgG fragments, host cell proteins, or host cell nucleic acids.

141. The method of claim 140, wherein the polypeptide is produced in a CHO cell and the impurity comprises CHO host cell protein (CHOP) and/or CHO nucleic acids.
